SEQUENCE LISTING (188 sequences)

SEQ ID NO: 1 - DNA, 1614 bp, Homo sapiens
  CDS (58)..(1557)
ggtctctttggcttcagaaattaccaagaaagcctggaccccgggtgaaacggagaaatggacacaagtcccctgtgtttctccattctgttggttttatgcatctttatccaatcaagtgcccttggacaaagcctgaaaccagagccatttggaagaagagctcaagctgttgaaacaaacaaaacgctgcatgagatgaagaccagattcctgctctttggagaaaccaatcagggctgtcagattcgaatcaatcatccggacacgttacaggagtgcggcttcaactcctccctgcctctggtgatgataatccacgggtggtcggtggacggcgtgctagaaaactggatctggcagatggtggccgcgctgaagtctcagccggcccagccagtgaacgtggggctggtggactggatcaccctggcccacgaccactacaccatcgccgtccgcaacacccgccttgtgggcaaggaggtcgcggctcttctccggtggctggaggaatctgtgcaactctctcgaagccatgttcacctaattgggtacagcctgggtgcacacgtgtcaggatttgccggcagttccatcggtggaacgcacaagattgggagaatcacagggctggatgccgcgggacctttgtttgagggaagtgcccccagcaatcgtctttctccagatgatgccaattttgtggatgccattcatacctttacccgggagcacatgggcctgagcgtgggcatcaaacagcccataggacactatgacttctatcccaacgggggctccttccagcctggctgccacttcctagagctctacagacatattgcccagcacggcttcaatgccatcacccagaccataaaatgctcccacgagcgatcggtgcaccttttcatcgactccttgctgcacgccggcacgcagagcatggcctacccgtgtggtgacatgaacagcttcagccagggcctgtgcctgagctgcaagaagggccgctgcaacacgctgggctaccacgtccgccaggagccgcggagcaagagcaagaggctcttcctcgtaacgcgagcccagtcccccttcaaagtttatcattaccagttcaagatccagttcatcaaccaaactgagacaccaatacaaacaacttttaccatgtcactactcggaacaaaagagaaaatgcagaaaattcccatcactctgggcaaaggaattgctagtaataaaacgtattcctttcttatcacgctggatgtggatatcggcgagctgatcatgatcaagttcaagtgggaaaacagtgcagtgtgggccaatgtctgggacacggtccagaccatcatcccatggagcacagggccgcgccactcaggcctcgttctgaagacgatcagagtcaaagcaggagaaacccagcaaagaatgacattttgttcagaaaacacagatgacctactacttcgcccaacccaggaaaaaatcttcgtgaaatgtgaaataaagtctaaaacatcaaagcgaaagatcagatgagatttaatgaagacccagtgtaaagaataaatgaatcttactccttaaaaaaaaaaa

SEQ ID NO: 2 - protein, 499 aa, Homo sapiens
MDTSPLCFSILLVLCIFIQSSALGQSLKPEPFGRRAQAVETNKTLHEMKTRFLLFGETNQGCQIRINHPDTLQECGFNSSLPLVMIIHGWSVDGVLENWIWQMVAALKSQPAQPVNVGLVDWITLAHDHYTIAVRNTRLVGKEVAALLRWLEESVQLSRSHVHLIGYSLGAHVSGFAGSSIGGTHKIGRITGLDAAGPLFEGSAPSNRLSPDDANFVDAIHTFTREHMGLSVGIKQPIGHYDFYPNGGSFQPGCHFLELYRHIAQHGFNAITQTIKCSHERSVHLFIDSLLHAGTQSMAYPCGDMNSFSQGLCLSCKKGRCNTLGYHVRQEPRSKSKRLFLVTRAQSPFKVYHYQFKIQFINQTETPIQTTFTMSLLGTKEKMQKIPITLGKGIASNKTYSFLITLDVDIGELIMIKFKWENSAVWANVWDTVQTIIPWSTGPRHSGLVLKTIRVKAGETQQRMTFCSENTDDLLLRPTQEKIFVKCEIKSKTSKRKIR

SEQ ID NO: 3 - DNA, 7390 bp, Homo sapiens
  CDS (199)..(1137)
cggaactcgcgggttcggagccgcccgctgaggtcagaaggaggcgtctgcgctgatcgggtccgccgcgcgccagagccagagtcgcagccgaggggagccggggccggagcccgagcccgagccgagccggagcccgagcgagcggcggagaccgtgcccccgcctcggccccgcgccgccgcggccaggcccggcatggaggaggagtgccgggtgctctccatacagagccacgtcatccgcggctacgtgggcaaccgggcggccacgttcccgctgcaggttttgggatttgagattgacgcggtgaactctgtccagttttcaaaccacacaggctatgcccactggaagggccaagtgctgaattcagatgagctccaggagttgtacgaaggcctgaggctgaacaacatgaataaatatgactacgtgctcacaggttatacgagggacaagtcgttcctggccatggtggtggacattgtgcaggagctgaagcagcagaaccccaggctggtgtacgtgtgtgatccagtcttgggtgacaagtgggacggcgaaggctcgatgtacgtcccggaggacctccttcccgtctacaaagaaaaagtggtgccgcttgcagacattatcacgcccaaccagtttgaggccgagttactgagtggccggaagatccacagccaggaggaagccttgcgggtgatggacatgctgcactctatgggccccgacaccgtggtcatcaccagctccgacctgccctccccgcagggcagcaactacctgattgtgctggggagtcagaggaggaggaatcccgctggctccgtggtgatggaacgcatccggatggacattcgcaaagtggacgccgtctttgtgggcactggggacctgtttgctgccatgctcctggcgtggacacacaagcaccccaataacctcaaggtggcctgtgagaagaccgtgtctaccttgcaccacgttctgcagaggaccatccagtgtgcaaaagcccaggccggggaaggagtgaggcccagccccatgcagctggagctgcggatggtgcagagcaaaagggacatcgaggacccagagatcgtcgtccaggccacggtgctgtgagggccccgccgcttgcccgtgacacgcagcgcgttggtgtctccgtgtttgtccctgtgaaaacatgtaacgtctgccttagagccatgaccgaaacttgatatttttttctttcatgagtgtccggcatctgctggtcttcattgtgaaacgtgccagtcgtgctttgtgaaaaataacaaagtggtcacagaaatttgtgatctgaaaacccggctcccttccccacaaggctcctgggcctccgggaagacgggcccctgtttgccatctcgggggtgttccctgtgggagggtgagtgggtgaggccgagcctgctgcgtgtggagcctcgagtgggccctggctgccactaccgtacagaggccgtgtcgcgctgggctgggcctgggtggcctctgtctttgcatctctgagaaggagtcgggtggtaacggttggggtcaggaagaattctgccaagtatctttactgtcattctgaccatagcctctttgttcccgcattcgaacttttggttcttactttgctgctcgtttagtccctggggatttcagatcttaggctgttgtttcaccgtatgggagggttgatgtgagcttgcttggagacacacggtgcagcatcagggaccttcccaggccccagcaaattcaagtcggtctgcagacctctcagctacccgcgggacctcttgtaacccatcggcatcttccaggaatccgccgagtgacttgaggaagatgctaacgcagtaaggtctgtgctgggccaagagcagctttgaagctccagagaaccaccccgtcaggttccttgtggaagctcccctcatccgtggtgcagcaggctgagcactgcgcgtttgccacgtgctgcccgtgacagcacattgagccacagcatttgtagacaggacagaggggtgcctgccccctgcccctgctggcacatttaacccttgtcccctgacctcagttctgtgccccaccaaatgcccaggggcaagaggccaccctggaagctgccaatcttccaaggtgggtgtggggcacggtgggggcgggcagctcccaggcccttgggcaggctggggtgacggcagaggccacagcaccagctctgacaagtcctatcatcctctgctcagcagtgacctccctggccccactttgcccagagtttggggtccccccaggtatagctataggcggcagtgcctgtccctggcctgccttgatttcagccacacccctgcagccctgcatcccagctctggggtgtgcagaggtttgtgtctccagggaacccacggctggagagaaatagggagatgcaggaagtgggggcccatggggcccccaagaagcggactctccaaggggtacccccaccccgctaccttccccacggacgggcccctcctggagcccataccctcctgtgaggccattccagtgtcttctagaaagactcgcttgccaggagtgcgttctttgttgaaaaatgccctgaagcgaaaagatgcaggtttatatggaacccccaccccctcccccactctcccactctgttcgttctgaatgtcttcacgagcgtgcatcagggcgcctggctcccccacctcagccagtgagtcagacacgggtttcgcagccatgtttcctggctccgaggacacgggtggcaggcccgttgcagcccagagccactggtccctacagggcgccgccacaccagcaggaaggaggatggctgtgtccggagcctggcggggaggcggcctccccagtatgtgagtgcagggatctgccagaaccacctggccctctgtagggcgtttaactggaaataccctcactgccaagtggagactggggcgtgtgccacattgccagccaccaggaaagcttttctttttcttttttttttttttttaaacaccaagagcacgtatagcatgggggaaagaacctaaatgtctctctgtcctgtgagctggtgaaaaacccagcatgagaacgcagtgtcaggtgtgggactccttctgcccctgcagtgggtgttacgggcggtgtgccctggcgagcaagctttgattcttggttctttgagctcgtttcagaggctgagtccccacatcagctttagttcttggacttccctgtattaagcaagaattaggagaatggctgtccctgcaggcgcctcccgtaaatcctgagctctctggcgcaatctgaaacttctcttctgttttctttggctgtatcagccgaaccaggagaggcctgggctgcgactaaggagaaagaaatcgggggtttctgagagcagatggtgcctttgtgggtgcagggcttttgtggaaattgtcagcctctacgggcagagtccggcatcccctccccagactgcctgctgtcaaaccacggagcagctggagcctgccctgtccacggcccgtttccacccgggcatgttcgtctctcatgacttcggcagaggcccctggtggccttcagtttcagtttctcatccaggaaggtaaccttgggcattggcagtgggtttccctatggcttggatccagattagaattgatctttgttttcactttccatagttaataacatgcaaaataatgagaagaatttattttaaggtgacagctatactggtccaacatcgcctgcttattgtcagggtacagaagtttaatactttcttaatccagtttttcaaacttctccctgtagaccgtaaggatgaattccacaataggatcctttttaaaatcgattttaaattgttgcctagtcctgccaaggttattatgtgcatctgttatttttccaatacatgtaaacagttgcagcatgatgctttgtttaatgtcctgttcttaagctcgttagagccagttttgaaacgtttggtcttaccgtgaacggaggctggcttggcttagccacgctgatgagtaagtgagggatgtctccatcttgagatcaccaggcaagagagttgcctgcaccaggtaagaggccaaagcccctggggtaacagtccccaccgctacccgaggtaaaacaataaaagctatgtggttgagctcaggcctctcgtgcctggtgtcagagaaggcagagcccacagtaggtgcacggtgcaaggccctgggagggcactggccagggaaggtggtatagatggccctcagattgcggggccccgagcagctccccactctgcccgtccaccttccctggctccagcctcattctctctttagtttaactatgcaaagagaggaggttgagagtgttctggcagctggagctcttttccttgtccttcctgccctccgatggggccacctgtgtcggggcagcagtgtccatgtttatggagatcagaggtgtccccactgtgtggctggactgtactctgctgcccgggtagccaggagtctctccctctctcccctgccgcctgcctggtctcatgggcctccttcacacacccctccctgtggatcgcctgcctgggcccagagcaggggaactggagtttgtgagtgagcagagcaggttatgtgcagacagggaaacgagaactttggacctggctttctgagtccaggtgagagctgtgtggccccccgatgccactctgcccgccggagggatgtgcctgctgagccttttccttccacgccgcctctcactgccaggccagcggcttccgctgagactcgctggagaggcggctcccgtgtccgtccaccgagcactcagatggatgctgatcaccagggccgagggggctcccagaaggaccccaggccctggggagggtggctgtgggaggccaagtccactgcccggaagtcttgtcagccctaagccagggaagcctggagcgtggcctggcgggtctgggtggacaccgtccccactccggactcccagcacaggggaggatacctgagcctgtatggccctgtagccctgggcagagctgggcctgtcgtgtgttcctgcctggcaggtgcaggtgctggccatctgcaggtggaaggaggtgggaatcttggattttttgtttttttttgtttttttttttttgagatgaagtctcgctctgacacccaggctggcgtgcagtggtgtgatctcggctcactgcaaactccgcttcctgggttcaagtggttctcctgccccagcctcccaagtagctgggattacaggcatgcgccaccacgctcagctgatttttgtatttttagtagagatggggtttcaccatgttggccaagctggtctcaaactcctgacctcaagtgatctgcccgcctcggcctcccagagtgctgggattacaggcatgagccagtgcacccggcggaatcttggaatttttatagacagcacctcagtttctgactccagccgcacacctcctgcctctgccagcaggggttgccgccagaccagagccagggccaggtccctgcgtccatcccccccggtaggatggacgtgagccatccttctaggggacttttttcagtgtgcgactcgtctctgttaggtggtaggagccagtttgtgtggcctgtgccacgctccacagtgcgtggctgggctctgtgtgtggcctgtgtcccctgtccctgcaggacccagcaggcatcgtggcgtgacagctgtgtccaagccactgcccgggcatcccatcacccaccagggtgcacggtctctcctgctgggggctttctgtcgcatgtgtgtctcctgtcgactctgcagtttgttctcagagcagaatgtttcctgttctcagtgcacaaagacactggttttcaatcggcgtctaaaaccacgttcctgcctttcattgcaacacggtgtgttcatttgtttaaaacagtttaatgagtaagtttagatgactggtcaatatcttaaaaatgtatattagtaagaagttcttcctggaatttttctttcgattctggcagaataaacaggtgtttttagttttcccactgtctgagccaagcaggaccctgtcccagagcaagagatgtccccttccatctctgacccttgcctgggacaagctttgatggggggccccagcttcaaggctgtggtgggaacagcacccccaaatgccagcctctcctttcttcccatccaccagtatactgcggggccatttctggtctttgtccaacaggaaacccatttctggtgggatatgccttccagtgccacagggccactcaccccatgcatctctgtcctgcccgtcagtgctgggacggacagcaagggcaagcccagtgtctggcggataggtgggtgggaacagagaggggagaatgccgtcctaagcttctgcttggggatcccccacacgacctgggtactgcctgggaaacctgtcctaagtaaaactatggacctcgcctcgcccaccggcctgcgaagccagcatctccgtgaaggtggatggaagcgcctttgtcctcattttgagctgcaagctgggtcagcggctctgaagccctcgagtgactttctaacccaagacccagcccctggcaggaggagggtgggtgcagggctggtgggacaaaaagaggcctcagcaggcctggaagacccttccagtacatcccacagcgtgtcgagcagctgggagaacctgtgtcaagctcgagccgtcataggtccccatgaggtgtctgaagccccttcttggtgatgggaggcagaggtgctgacgttctggagcatggacgtgagtcctcagctggctccgcgtgggcccttggagggtgccaggtgtgtggtgaccttctggatgcctttaacttcatggctgcgtcattcctgatttagaactttaaccggagcttcatctagtgattgcaaaactggaccaatgggaggacggcggcgcagcccgctccctccgtggaatggagctcagctcttcggaggcatcaaagcacctgtcgcctccgtggtccccctgctgagggagtgcggcctctgcaaggttcgggggtggcttcgtttgcctggagtggccggccctgcttgtgccatgtggatgtttgtgagcctcggtcctacagcactgtgtaggctgcatctgtttcgtgctggtcctgttgacttgtatgatatccacaaataaatattttcatggcggtcgtgttgaaaaaaaaaa

SEQ ID NO: 4 - protein, 312 aa, Homo sapiens
MEEECRVLSIQSHVIRGYVGNRAATFPLQVLGFEIDAVNSVQFSNHTGYAHWKGQVLNSDELQELYEGLRLNNMNKYDYVLTGYTRDKSFLAMVVDIVQELKQQNPRLVYVCDPVLGDKWDGEGSMYVPEDLLPVYKEKVVPLADIITPNQFEAELLSGRKIHSQEEALRVMDMLHSMGPDTVVITSSDLPSPQGSNYLIVLGSQRRRNPAGSVVMERIRMDIRKVDAVFVGTGDLFAAMLLAWTHKHPNNLKVACEKTVSTLHHVLQRTIQCAKAQAGEGVRPSPMQLELRMVQSKRDIEDPEIVVQATVL

SEQ ID NO: 5 - DNA, 21 bp, artificial sequence
  siACHE_1
cgccctctggctgcaaataaa

SEQ ID NO: 6 - DNA, 21 bp, artificial sequence
  siACHE_2
ccagagtgtctgctaccaata

SEQ ID NO: 7 - DNA, 21 bp, artificial sequence
  siADAR_1
tccctcgataacagtcagcta

SEQ ID NO: 8 - DNA, 21 bp, artificial sequence
  siADAR_2
ttccgttaccgcagggatcta

SEQ ID NO: 9 - DNA, 21 bp, artificial sequence
  siALDH7A1_1
tccgattctctatgtctttaa

SEQ ID NO: 10 - DNA, 21 bp, artificial sequence
  siALDH7A1_2
aaggatgattggaggacctat

SEQ ID NO: 11 - DNA, 21 bp, artificial sequence
  siALK_1
cacctacgtatttaagatgaa

SEQ ID NO: 12 - DNA, 21 bp, artificial sequence
  siALK_2
ctcgaccatcatgaccgacta

SEQ ID NO: 13 - DNA, 21 bp, artificial sequence
  siAPAF1_1
cagtgaaggtatggaatatta

SEQ ID NO: 14 - DNA, 21 bp, artificial sequence
  siAPAF1_2
taggcagagtataaagtatta

SEQ ID NO: 15 - DNA, 21 bp, artificial sequence
  siASTL_1
caggctttgaaatcaacttca

SEQ ID NO: 16 - DNA, 21 bp, artificial sequence
  siASTL_2
tcccatcttcagaagcaggaa

SEQ ID NO: 17 - DNA, 21 bp, artificial sequence
  siBAIAP3_1
gtcgaccttgctggacattaa

SEQ ID NO: 18 - DNA, 21 bp, artificial sequence
  siBAIAP3_2
ctcgcctgactccatccagaa

SEQ ID NO: 19 - RNA, 21 nt, artificial sequence
  siBAK1_1
gcgaagucuuugccuucucnn

SEQ ID NO: 20 - RNA, 21 nt, artificial sequence
  siBAX_1
ggugccggaacugaucagann

SEQ ID NO: 21 - RNA, 21 nt, artificial sequence
  siBCL2_1
gcugcaccugacgcccuucnn

SEQ ID NO: 22 - DNA, 21 bp, artificial sequence
  siBCL2L1_1
aaagtgcagttcagtaataaa

SEQ ID NO: 23 - DNA, 21 bp, artificial sequence
  siBCL2L1_2
caagctttcccagaaaggata

SEQ ID NO: 24 - DNA, 21 bp, artificial sequence
  siBMP7_1
cccggaagttcctgtaataaa

SEQ ID NO: 25 - DNA, 21 bp, artificial sequence
  siBMP7_2
ctcgtggaacatgacaaggaa

SEQ ID NO: 26 - DNA, 21 bp, artificial sequence
  siBSN_1
aaaggacttgatataaacaaa

SEQ ID NO: 27 - DNA, 21 bp, artificial sequence
  siBSN_2
cacgctgtgtcccatatgcaa

SEQ ID NO: 28 - DNA, 21 bp, artificial sequence
  siBTNL2_1
atggagggacatggaaggaaa

SEQ ID NO: 29 - DNA, 21 bp, artificial sequence
  siBTNL2_2
cagagtgggagaagatataca

SEQ ID NO: 30 - DNA, 21 bp, artificial sequence
  siC10ORF92_1
cagggcctattgtcacttcaa

SEQ ID NO: 31 - DNA, 21 bp, artificial sequence
  siC10ORF92_2
caggagaggcgaggacctgaa

SEQ ID NO: 32 - DNA, 21 bp, artificial sequence
  siC1ORF91_1
accgtctgtcttcacaaagta

SEQ ID NO: 33 - DNA, 21 bp, artificial sequence
  siC1ORF91_2
ctggcgagtagtagctgtcaa

SEQ ID NO: 34 - DNA, 21 bp, artificial sequence
  siC21ORF2_1
aagcgctttgttggaatggta

SEQ ID NO: 35 - DNA, 21 bp, artificial sequence
  siC21ORF2_2
ccgcatgaagctgacgcggaa

SEQ ID NO: 36 - RNA, 21 nt, artificial sequence
  siCASP2_1
acagcuguuguugagcgaann

SEQ ID NO: 37 - RNA, 21 nt, artificial sequence
  siCCBL1_1
ccaguacaccaagacauuunn

SEQ ID NO: 38 - RNA, 21 nt, artificial sequence
  siCCBL1_2
gguccagaucacaucaugann

SEQ ID NO: 39 - DNA, 21 bp, artificial sequence
  siCD151_1
caccctgtgccatcaccataa

SEQ ID NO: 40 - DNA, 21 bp, artificial sequence
  siCD151_2
ctgcctgtacaggagtctcaa

SEQ ID NO: 41 - DNA, 21 bp, artificial sequence
  siCD34_1
cacgtggtggctgataccgaa

SEQ ID NO: 42 - DNA, 21 bp, artificial sequence
  siCD34_2
aacggccattcagcaagacaa

SEQ ID NO: 43 - DNA, 21 bp, artificial sequence
  siCLIC1_1
aaggtgtctctcagaggaagt

SEQ ID NO: 44 - DNA, 21 bp, artificial sequence
  siCLIC1_2
ttcctgctgtatggcactgaa

SEQ ID NO: 45 - DNA, 21 bp, artificial sequence
  siCOMMD9_1
aagctcctgagtgcaacttaa

SEQ ID NO: 46 - DNA, 21 bp, artificial sequence
  siCOMMD9_2
cccgctgttctcactcatctt

SEQ ID NO: 47 - DNA, 21 bp, artificial sequence
  siCOX5B_1
cacctgcactaaattactcaa

SEQ ID NO: 48 - DNA, 21 bp, artificial sequence
  siCOX5B_2
aggcaccagggaagaccctaa

SEQ ID NO: 49 - DNA, 21 bp, artificial sequence
  siCOX7C_1
ctagatatgtttgtcaataaa

SEQ ID NO: 50 - DNA, 21 bp, artificial sequence
  siCOX7C_2
caagtggtcgttactagctaa

SEQ ID NO: 51 - DNA, 21 bp, artificial sequence
  siCYP11B2_1
cactagatcatgggatcctaa

SEQ ID NO: 52 - DNA, 21 bp, artificial sequence
  siCYP11B2_2
aaggcggaactgtcactagaa

SEQ ID NO: 53 - DNA, 21 bp, artificial sequence
  siDEFB4_1
gccctagaaggtataaacaaa

SEQ ID NO: 54 - DNA, 21 bp, artificial sequence
  siDEFB4_2
tgccttaagagtggagccata

SEQ ID NO: 55 - DNA, 21 bp, artificial sequence
  siDHRSX_1
ccgcgttaatacaactgagta

SEQ ID NO: 56 - DNA, 21 bp, artificial sequence
  siDHRSX_2
tgccctgataagcaacttaaa

SEQ ID NO: 57 - DNA, 21 bp, artificial sequence
  siEBP_1
actggacaactttgtacctaa

SEQ ID NO: 58 - DNA, 21 bp, artificial sequence
  siEBP_2
cacagtgtgcatggaaaccat

SEQ ID NO: 59 - DNA, 21 bp, artificial sequence
  siEEF2_1
ccgcgccatcatggacaagaa

SEQ ID NO: 60 - DNA, 21 bp, artificial sequence
  siEEF2_2
tgccgtttctttcaatattta

SEQ ID NO: 61 - DNA, 21 bp, artificial sequence
  siFBXO42_1
ctgggatcaaatctccattaa

SEQ ID NO: 62 - DNA, 21 bp, artificial sequence
  siFBXO42_2
ctcctgtgtgatagatgataa

SEQ ID NO: 63 - DNA, 21 bp, artificial sequence
  siFLOT2_1
ctgcctgtcccttctggtaaa

SEQ ID NO: 64 - DNA, 21 bp, artificial sequence
  siFLOT2_2
caccaagattgctgactctaa

SEQ ID NO: 65 - DNA, 21 bp, artificial sequence
  siFN3KRP_1
ccggactagcttaagaccaat

SEQ ID NO: 66 - DNA, 21 bp, artificial sequence
  siFN3KRP_2
acgagtgttcgtgaaagtgaa

SEQ ID NO: 67 - DNA, 21 bp, artificial sequence
  siGRLF1_1
caggatgttctgggagaggaa

SEQ ID NO: 68 - DNA, 21 bp, artificial sequence
  siGRLF1_2
caccaccgaagaggtgtttaa

SEQ ID NO: 69 - DNA, 21 bp, artificial sequence
  siGUCA1A_1
caccgatacagtgttctccaa

SEQ ID NO: 70 - DNA, 21 bp, artificial sequence
  siGUCA1A_2
gccggtcgttgtggactctaa

SEQ ID NO: 71 - DNA, 21 bp, artificial sequence
  siHDLBP_1
aaggatctaatcattgagcaa

SEQ ID NO: 72 - DNA, 21 bp, artificial sequence
  siHDLBP_2
atccgtgaaattcgtgacaaa

SEQ ID NO: 73 - DNA, 21 bp, artificial sequence
  siHGD_1
accctacaagtacaacctgaa

SEQ ID NO: 74 - DNA, 21 bp, artificial sequence
  siHGD_2
cgggaattatacaccctacaa

SEQ ID NO: 75 - DNA, 21 bp, artificial sequence
  siHIRIP3_1
taggaagaaacctgtggtaaa

SEQ ID NO: 76 - DNA, 21 bp, artificial sequence
  siHIRIP3_2
cagccaaagaggagaatccaa

SEQ ID NO: 77 - DNA, 21 bp, artificial sequence
  siIAPP_1
ttagaggacaatgtaactcta

SEQ ID NO: 78 - DNA, 21 bp, artificial sequence
  siIAPP_2
atgcagggtattgcgaaacaa

SEQ ID NO: 79 - DNA, 21 bp, artificial sequence
  siIL6R_1
acccagttagctctcaagtta

SEQ ID NO: 80 - DNA, 21 bp, artificial sequence
  siIL6R_2
ctgcgggaccatggagtggta

SEQ ID NO: 81 - DNA, 21 bp, artificial sequence
  siITGB6_1
ctggaattacttgtcagccca

SEQ ID NO: 82 - DNA, 21 bp, artificial sequence
  siITGB6_2
aacccttgcagtagtattcca

SEQ ID NO: 83 - DNA, 21 bp, artificial sequence
  siKHSRP_1
caagaggagattgaaactgaa

SEQ ID NO: 84 - DNA, 21 bp, artificial sequence
  siKHSRP_2
caggattcaggctgcaaagta

SEQ ID NO: 85 - DNA, 21 bp, artificial sequence
  siKIA1161_1
caggccagacttcgtgcctta

SEQ ID NO: 86 - DNA, 21 bp, artificial sequence
  siKIA1161_2
cgcggccgccatcaaagtcaa

SEQ ID NO: 87 - DNA, 21 bp, artificial sequence
  siLIPC_1
caggagaaacccagcaaagaa

SEQ ID NO: 88 - DNA, 21 bp, artificial sequence
  siLIPC_2
ctgaagacgatcagagtcaaa

SEQ ID NO: 89 - DNA, 21 bp, artificial sequence
  siLOC388882_1
caccttggtgagaatcaggaa

SEQ ID NO: 90 - DNA, 21 bp, artificial sequence
  siLOC388882_2
ctgctgtacatgacatctcta

SEQ ID NO: 91 - DNA, 21 bp, artificial sequence
  siLOC389727_1
ccacgcagagatgacagccaa

SEQ ID NO: 92 - DNA, 21 bp, artificial sequence
  siLOC389727_2
ctggcggtgaatgactttgaa

SEQ ID NO: 93 - DNA, 21 bp, artificial sequence
  siLOC390533_1
cgggtggtatggccaaagcat

SEQ ID NO: 94 - DNA, 21 bp, artificial sequence
  siLOC390533_2
atacaacagcttcttaggtaa

SEQ ID NO: 95 - DNA, 21 bp, artificial sequence
  siLOC440056_1
cccgaggcttcgctccgcaaa

SEQ ID NO: 96 - DNA, 21 bp, artificial sequence
  siLOC440056_2
ctccggcgcgagcctcccaaa

SEQ ID NO: 97 - DNA, 21 bp, artificial sequence
  siLOC440611_1
caccgaaagagtgtttccaaa

SEQ ID NO: 98 - DNA, 21 bp, artificial sequence
  siLOC440611_2
aaagagtgtttcaaacgtgaa

SEQ ID NO: 99 - DNA, 21 bp, artificial sequence
  siLOC441115_1
cagagctacctcaaacaggaa

SEQ ID NO: 100 - DNA, 21 bp, artificial sequence
  siLOC441115_2
agggaggtctttgaaagttca

SEQ ID NO: 101 - DNA, 21 bp, artificial sequence
  siLOC442126_1
cagggcgggcgcagtcctgga

SEQ ID NO: 102 - DNA, 21 bp, artificial sequence
  siLOC442126_2
tcgcacgagcgggatactcca

SEQ ID NO: 103 - DNA, 21 bp, artificial sequence
  siLRMP_1
ctgggaaagtatagcatgaaa

SEQ ID NO: 104 - DNA, 21 bp, artificial sequence
  siLRMP_2
cacaaactatatggtggtcaa

SEQ ID NO: 105 - DNA, 21 bp, artificial sequence
  siMAT2A_1
cagcaggatcctgatgccaaa

SEQ ID NO: 106 - DNA, 21 bp, artificial sequence
  siMAT2A_2
cagggagtgttccctatccaa

SEQ ID NO: 107 - DNA, 21 bp, artificial sequence
  siMCL1_1
ctggtttggcatatctaataa

SEQ ID NO: 108 - DNA, 21 bp, artificial sequence
  siMCL1_2
cgggactggctagttaaacaa

SEQ ID NO: 109 - DNA, 21 bp, artificial sequence
  siMPP6_1
tggaaggtgtactaatatata

SEQ ID NO: 110 - DNA, 21 bp, artificial sequence
  siMPP6_2
ctgatgattcagtaaggttaa

SEQ ID NO: 111 - DNA, 21 bp, artificial sequence
  siNANOS1_1
caggcagtgctcaaactagaa

SEQ ID NO: 112 - DNA, 21 bp, artificial sequence
  siNANOS1_2
caccagtttacccataagcaa

SEQ ID NO: 113 - DNA, 21 bp, artificial sequence
  siNCKAP1_1
acgcatgaactatgtccagaa

SEQ ID NO: 114 - DNA, 21 bp, artificial sequence
  siNCKAP1_2
caggcatatactagtgtctca

SEQ ID NO: 115 - DNA, 21 bp, artificial sequence
  siNCOA3_1
caggaggagattataatactt

SEQ ID NO: 116 - DNA, 21 bp, artificial sequence
  siNCOA3_2
ccgacaggcacttgaattgaa

SEQ ID NO: 117 - DNA, 21 bp, artificial sequence
  siNLRP1_1
cagcttctgctcgccaataaa

SEQ ID NO: 118 - DNA, 21 bp, artificial sequence
  siNLRP1_2
ctggtttgccttccagcacta

SEQ ID NO: 119 - DNA, 21 bp, artificial sequence
  siOSTCL_1
aagcattggctctatgactga

SEQ ID NO: 120 - DNA, 21 bp, artificial sequence
  siOSTCL_2
aaaggctgtccaatcctctaa

SEQ ID NO: 121 - DNA, 21 bp, artificial sequence
  siOVCH1_1
tacgaggtgcatttggtataa

SEQ ID NO: 122 - DNA, 21 bp, artificial sequence
  siOVCH1_2
agcgtgtaatactgtgctcaa

SEQ ID NO: 123 - DNA, 21 bp, artificial sequence
  siPDXK_1
tcgagtgactttctaacccaa

SEQ ID NO: 124 - DNA, 21 bp, artificial sequence
  siPDXK_2
ccctgtattaagcaagaatta

SEQ ID NO: 125 - RNA, 21 nt, artificial sequence
  siPDXP_1
gguuugagggcccuugcaann

SEQ ID NO: 126 - RNA, 21 nt, artificial sequence
  siPDXP_2
gugauagugacucaucaaunn

SEQ ID NO: 127 - DNA, 21 bp, artificial sequence
  siPLD3_1
ttgagggaagatgaagcctaa

SEQ ID NO: 128 - DNA, 21 bp, artificial sequence
  siPLD3_2
ccgcatggtggacatgcagaa

SEQ ID NO: 129 - DNA, 21 bp, artificial sequence
  siPLK1S1_1
aaggaaatatgtgaatctgaa

SEQ ID NO: 130 - DNA, 21 bp, artificial sequence
  siPLK1S1_2
cagcggtgcattcgagacaaa

SEQ ID NO: 131 - DNA, 21 bp, artificial sequence
  siPPM1B_1
caaccaagtgtttagaatgaa

SEQ ID NO: 132 - DNA, 21 bp, artificial sequence
  siPPM1B_2
tagcctaactacacacatcaa

SEQ ID NO: 133 - DNA, 21 bp, artificial sequence
  siPPM1J_1
aacgagatggataaaggtgaa

SEQ ID NO: 134 - DNA, 21 bp, artificial sequence
  siPPM1J_2
agggatgtccgctatatccaa

SEQ ID NO: 135 - DNA, 21 bp, artificial sequence
  siPRSS21_1
ccactttgagtggatccagaa

SEQ ID NO: 136 - DNA, 21 bp, artificial sequence
  siPRSS21_2
acccttggcctgtaacaagaa

SEQ ID NO: 137 - DNA, 21 bp, artificial sequence
  siPSMC5_1
ttggtcaaggtacatcctgaa

SEQ ID NO: 138 - DNA, 21 bp, artificial sequence
  siPSMC5_2
ccgggtggctctaaggaatga

SEQ ID NO: 139 - DNA, 21 bp, artificial sequence
  siPSMD7_1
ttccgtattggtcatcattga

SEQ ID NO: 140 - DNA, 21 bp, artificial sequence
  siPSMD7_2
caggccctaaactacacaaga

SEQ ID NO: 141 - DNA, 21 bp, artificial sequence
  siRAB3C_1
gtggcaaatatgtgatcttaa

SEQ ID NO: 142 - DNA, 21 bp, artificial sequence
  siRAB3C_2
aaggacatttgccaacaatga

SEQ ID NO: 143 - DNA, 21 bp, artificial sequence
  siRAB40A_1
atgggaggaagaaatagtaca

SEQ ID NO: 144 - DNA, 21 bp, artificial sequence
  siRAB40A_2
taggctgaataatctcctgta

SEQ ID NO: 145 - DNA, 21 bp, artificial sequence
  siRAC2_1
aaggagattgactcggtgaaa

SEQ ID NO: 146 - DNA, 21 bp, artificial sequence
  siRAC2_2
caatgtgatggtggacagcaa

SEQ ID NO: 147 - DNA, 21 bp, artificial sequence
  siRNF26_1
cactagggtccaaatacagaa

SEQ ID NO: 148 - DNA, 21 bp, artificial sequence
  siRNF26_2
gaccttggtgttggacctcaa

SEQ ID NO: 149 - DNA, 21 bp, artificial sequence
  siRPSAP32_1
cactactgaaatcctcactaa

SEQ ID NO: 150 - DNA, 21 bp, artificial sequence
  siRPSAP32_2
ctgactatcattactctatga

SEQ ID NO: 151 - DNA, 21 bp, artificial sequence
  siRRAD_1
cgcggtggtctttgactgcaa

SEQ ID NO: 152 - DNA, 21 bp, artificial sequence
  siRRAD_2
cccggacgcgatgaccctgaa

SEQ ID NO: 153 - DNA, 21 bp, artificial sequence
  siSEMA3C_1
cagaatatgattcactattta

SEQ ID NO: 154 - DNA, 21 bp, artificial sequence
  siSEMA3C_2
tccctgaatattaacaatata

SEQ ID NO: 155 - DNA, 21 bp, artificial sequence
  siSEMG2_1
caggtaacaattcatagtcaa

SEQ ID NO: 156 - DNA, 21 bp, artificial sequence
  siSEMG2_2
aaggcagtatttcgatccaaa

SEQ ID NO: 157 - DNA, 21 bp, artificial sequence
  siSLC22A18AS_1
ctgcagcaacatagagtacaa

SEQ ID NO: 158 - DNA, 21 bp, artificial sequence
  siSLC22A18AS_2
ctcagccagtcttaaaggcaa

SEQ ID NO: 159 - DNA, 21 bp, artificial sequence
  siSLC39A5_1
ctcctgggacctcgtctacta

SEQ ID NO: 160 - DNA, 21 bp, artificial sequence
  siSLC39A5_2
caggcttctgttgctggacca

SEQ ID NO: 161 - DNA, 21 bp, artificial sequence
  siSOCS6_1
ttgatctaattgagcattcaa

SEQ ID NO: 162 - DNA, 21 bp, artificial sequence
  siSOCS6_2
tagaatcgtgaattgacataa

SEQ ID NO: 163 - DNA, 21 bp, artificial sequence
  siSTAT3_1
ctggtcttaactctgattgta

SEQ ID NO: 164 - DNA, 21 bp, artificial sequence
  siTCTE3_1
tagaatggagccattgaagaa

SEQ ID NO: 165 - DNA, 21 bp, artificial sequence
  siTCTE3_2
ctcaattgctgatataggtaa

SEQ ID NO: 166 - DNA, 21 bp, artificial sequence
  siTFF1_1
caccatggagaacaaggtgat

SEQ ID NO: 167 - DNA, 21 bp, artificial sequence
  siTFF1_2
accatcgacgtccctccagaa

SEQ ID NO: 168 - DNA, 21 bp, artificial sequence
  siTMED1_1
ccggccaaagtctaggcagaa

SEQ ID NO: 169 - DNA, 21 bp, artificial sequence
  siTMED1_2
gaggagatgctggatgttaaa

SEQ ID NO: 170 - RNA, 21 nt, artificial sequence
  siTP53_1
gacuccagugguaaucuacnn

SEQ ID NO: 171 - DNA, 21 bp, artificial sequence
  siTRIP4_1
cagaaatcaggcgaccatcta

SEQ ID NO: 172 - DNA, 21 bp, artificial sequence
  siTRIP4_2
cagctgcgaatccaggatcaa

SEQ ID NO: 173 - DNA, 21 bp, artificial sequence
  siUBE2L3_1
accaccgaagatcacatttaa

SEQ ID NO: 174 - DNA, 21 bp, artificial sequence
  siUBE2L3_2
aaggagcagcaccaaatccaa

SEQ ID NO: 175 - DNA, 21 bp, artificial sequence
  siUBE2T_1
tcgcaactgtgttgacctcta

SEQ ID NO: 176 - DNA, 21 bp, artificial sequence
  siUBE2T_2
aagagagagctgcacatgtta

SEQ ID NO: 177 - RNA, 21 nt, artificial sequence
  siUNR
gccgguaugccgguuaagunn

SEQ ID NO: 178 - DNA, 21 bp, artificial sequence
  siUSP8_1
caggttcaggcaagccattta

SEQ ID NO: 179 - DNA, 21 bp, artificial sequence
  siUSP8_2
aaggctcgtattcatgcagaa

SEQ ID NO: 180 - RNA, 21 nt, artificial sequence
  siVDAC1_1
guacggccugacguuuacann

SEQ ID NO: 181 - DNA, 21 bp, artificial sequence
  siWSB2_1
cacggcttcttacgataccaa

SEQ ID NO: 182 - DNA, 21 bp, artificial sequence
  siWSB2_2
cacgttaattcggaagctaga

SEQ ID NO: 183 - DNA, 21 bp, artificial sequence
  siZDHHC9_1
aaccagattgtgaaactgaaa

SEQ ID NO: 184 - DNA, 21 bp, artificial sequence
  siZDHHC9_2
agcaggaatggcagtaataaa

SEQ ID NO: 185 - DNA, 21 bp, artificial sequence
  siZNF878_1
aagcattatcttatcttgtaa

SEQ ID NO: 186 - DNA, 21 bp, artificial sequence
  siZNF878_2
tagagttgcctcacaacttaa

SEQ ID NO: 187 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 188 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

(19) United States
(12) Patent Application Publication
(10) Pub. No.: US 2013/0281313 A1
(43) Pub. Date: Oct. 24, 2013

(54) PROGNOSTIC AND/OR PREDICTIVE BIOMARKERS AND BIOLOGICAL APPLICATIONS THEREOF

(71) Applicants: Lorenzo Galluzzi, Paris (FR); Annick Harel-Bellan, Paris (FR); Guido Kroemer, Paris (FR); Ken Olaussen, Paris (FR); Jean-Charles Soria, Igny (FR)

(72) Inventors: Lorenzo Galluzzi, Paris (FR); Annick Harel-Bellan, Paris (FR); Guido Kroemer, Paris (FR); Ken Olaussen, Paris (FR); Jean-Charles Soria, Igny (FR)

(73) Assignee: INSTITUT GUSTAVE ROUSSY, VILLEJUIF CEDEX (FR)

(21) Appl. No.: 13/819,157

(22) PCT Filed: Sep. 1, 2011
(86) PCT No.: PCT/EP11/65087

§ 371 Date: Jun. 19, 2013

(30) Foreign Application Priority Data
Sep. 1, 2010 (EP) 10305937.4

(51) Int. Cl. C12Q 1/68; G01N 33/50; G01N 33/574

(52) U.S. Cl. CPC C12Q 1/6886; G01N 33/57496; G01N 33/5091; G01N 33/5011
USPC 506/9; 435/6.11; 435/7.4; 436/104; 506/16; 435/6.13; 436/96

(57) ABSTRACT

The present invention relates to the fields of genetics, immunology and medicine. The present invention more specifically relates to the identification of human genes and expression products thereof which can be used to assess the prognosis of a cancer in a subject, to assess the sensitivity of a subject to a treatment of cancer, or monitor, in particular determine the efficacy, of such a treatment of cancer after a given period of time. Said human genes and expression products can further be used (i) in the prevention or treatment of cancer, in particular to determine or select the appropriate cancer treatment for a given subject and/or for a particular tumor, as well as (ii) for the screening of therapeutically active drugs. Particular compounds capable to compensate, in a subject, an abnormal expression of one of the herein described products, in particular when the subject is exposed to a therapeutic treatment of cancer, thereby allowing or improving its efficiency in the subject, are also herein disclosed. Inventors, in addition, provide kits and DNA chips usable in the context of the present invention.

The present disclosure generally relates to the fields of genetics, immunology and medicine. The inventors more particularly disclose the identification of human genes and expression products thereof which can be used to assess the prognosis of a cancer in a subject, to assess the sensitivity of a subject to a treatment of cancer, or monitor, in particular determine the efficacy, of such a treatment of cancer after a given period of time. Said human genes and expression products can further be used (i) in the prevention or treatment of cancer, in particular to determine or select the appropriate cancer treatment for a given subject and/or for a particular tumor, as well as (ii) for the screening of therapeutically active drugs. The present disclosure further describes particular compounds capable to compensate, in a subject, an abnormal expression of one of the herein described products, in particular when the subject is exposed to a therapeutic treatment of cancer thereby allowing or improving its efficiency in the subject. Inventors in addition provide kits and DNA chips usable in the context of the present invention.

### BACKGROUND ART

Cancer is the major cause of mortality in most industrialized countries. Non-small cell lung cancer (NSCLC), in particular, is one of the leading causes of cancer-related death among males, both in industrialized and developing countries {Beers, 2008; Jemal, 2008}. In spite of encouraging progress in developing personalized treatments, only a small fraction of NSCLC patients bear tumors whose specific characteristics allow them to benefit from therapies targeting specific growth receptors (such as the epidermal growth factor receptor, EGFR) {Kancha, 2009} or signal transducers (such as the anaplastic lymphoma kinase, ALK) {Soda, 2007}. Most NSCLC patients receive cytotoxic chemotherapeutics including platinum-based agents such as cisplatin (cis-diammineplatinum(II) dichloride, CDDP) and carboplatin {Cosaert, 2002; Seve, 2005} yielding highly heterogeneous therapeutic responses.

CDDP is used to treat various types of carcinomas (e.g., lung and ovarian cancer), sarcomas, lymphomas, and germ cell tumors. CDDP is a DNA damaging agent that induces 1,2-intrastrand d(GpG), 1,2-intrastrand d(ApG) and 1,3-intrastrand d(GpXpG) adducts. This latter type of adduct is readily excised by the nucleotide excision repair (NER) {Martin, 2008}, and the NER rate likewise determines the efficacy of CDDP-based chemotherapy in vivo. Thus, the expression level of ERCC1, one of the principal enzymes involved in NER, is predictive of the response to CDDP in NSCLC. Thus, patients with completely resected ERCC1-negative NSCLC benefit from adjuvant CDDP-based chemotherapy, whereas patients with ERCC1-positive tumors do not {Olaussen, 2006}.

Nonetheless, the response to CDDP is determined by multiple factors beyond NER. For instance, the ABC transporter breast cancer resistance protein (BCRP) can mediate CDDP resistance by increasing the efflux of CDDP from cells {Ota, 2009}. Downstream of the DNA damage response, the expression level of cell cycle blockers (such as the cyclin-dependent kinase inhibitor p^{27KIP1}) {Osoegawa, 2004}, and that of anti-apoptotic proteins (such as survivin and SMAC) may influence the clinical response to “adjuvant chemotherapy” (this expression being herein understood as a therapy applied to a subject having a tumor after surgical resection of at least part of said tumor) {Dai, 2010}. Moreover, CDDP can trigger apoptotic changes in enucleated cells, indicating the existence of cytoplasmic structures that are targeted by CDDP {Mandic, 2003}. Other factors such as the presence of stem cell-like CD133⁺ NSCLC cells may have a negative effect on the CDDP response rate {Bertolini, 2009}.

Biomarkers that provide prognostic information and predict the response of patients to chemotherapy are important in guiding therapeutic decisions. As an example, ERCC1-positivity of NSCLC tumors does not only predict the absence of any therapeutic benefit of CDDP-based chemotherapy, but also actually correlates with a detrimental outcome of chemotherapy. Treated individuals die earlier than untreated ones {Olaussen, 2006}. However, the clinical impact of ERCC1 measurements is marginal and controversial {Breen, 2008}, implying that more accurate biomarkers are urgently awaited.

There is an increasing need to identify or distinguish those patients who will respond to an existing treatment of cancer from those who will not.

Solutions to detect dysfunctions responsible for an absent or reduced response to existing treatments of cancer, in particular chemotherapeutic treatments, as well as compounds usable to overcome said dysfunctions and/or modify the response to conventional treatments of cancer further appear critical for the patient and are herein advantageously provided by inventors.

### SUMMARY

The present invention is based on the discovery by inventors of novel anti-cancer agent response modifiers (also herein identified as modifiers of the chemotherapeutic response) based on a genome-wide siRNA screen.

Inventors herein provide an extensive phenotypic, mechanistic and epistatic analysis of these factors and disclose advantageous biological applications thereof, in particular in the context of cancer prevention or treatment. Personalized cancer therapy in particular relies on biomarkers capable of predicting the evolution of an individual tumor as well as its response to a conventional treatment of cancer. Advantageous tools usable in the “personalized” follow-up, treatment and cure of a subject having a cancer are herein disclosed.

Inventors herein notably demonstrate that the expression of proteins that are involved in intermediate metabolism, a vitamin B6-activating enzyme and a triglyceride lipase in particular, influence responses to chemotherapeutic agents, such as in particular cisplatin (CDDP), in vitro and in vivo, in suitable experimental systems. Such proteins are herein identified as cisplatin response modifiers (CRM). The level of expression of these proteins can further be used to predict the fate of patients suffering from a cancer or, in other words, to assess the prognosis of a cancer in patients, in particular a cancer selected from non-small cell lung cancer (NSCLC), small cell lung cancer (SCLC), head and neck cancer, cervical carcinoma, ovarian cancer, osteosarcoma, melanoma and colorectal cancer, in particular non-small cell lung cancer (NSCLC).

An in vitro or ex vivo method of diagnosing, assessing or monitoring the sensitivity of a subject having a tumor to a treatment of cancer (chemotherapy for example) is herein described. Such a method may advantageously be used to avoid or stop a useless treatment. Such a method of the invention preferably comprises a step of determining, in a biological sample of said subject, the absence, presence or expression level of the expression product of at least one gene selected from the genes herein identified in Table I (see below), or a step of detecting an alteration, such as a single nucleotide polymorphism (SNP), in at least one of said genes, thereby assessing or monitoring whether the subject having a tumor is responsive or resistant to the treatment of cancer. In a particular embodiment, the treatment of cancer is a conventional therapeutic treatment of cancer, preferably a chemotherapeutic treatment of cancer, even more preferably a chemotherapy using a drug selected from an alkylating agent such as camptothecin, cyclophosphamide, mechlorethamine, uramustine, melphalan, chlorambucil, ifosfamide, carmustine, lomostine, streoptozocin, busulfan, thiotepa, dacarbazine, mitozolomide, temozolomide, procarbazine, altretamine, dacarbazine, or an alkylating-like agent, in particular a platinum-based agent selected from cis-platinum (cisplatin or CDDP), carboplatin, nedaplatin, satraplatin and oxali-platinum (oxaliplatin or OXP), most preferably a platinum-based agent, in particular CDDP.

If the subject is identified, using a method according to the present invention, as resistant to a particular treatment of cancer, the method advantageously further comprises a step of selecting a “compensatory molecule”, to be used, alone or in combination with the particular treatment of cancer (typically a conventional treatment of cancer as herein defined) as the appropriate therapeutic treatment of cancer for the subject.

A method of selecting an appropriate, preferably optimal, therapeutic treatment of cancer for a subject having a tumor, is therefore in addition herein described, as well as compensatory molecules for use in such a treatment of cancer, preferably in combination with a conventional treatment of cancer, in particular a chemotherapeutic treatment of cancer, in a subject identified, using a method as herein described, as resistant to the conventional treatment of cancer.

In a particular embodiment, the method of selecting an appropriate treatment of cancer for a subject having a tumor, comprises a step of determining presence, absence or expression level of the hepatic lipase (LIPC) in a biological sample of said subject, the absence of LIPC in the biological sample being the indication that a chemotherapy will be efficient in the subject, the presence of LIPC in the biological sample, or an over expression thereof when compared to a reference expression level, being, on the contrary, the indication that a chemotherapy, in particular a chemotherapy using cis-platinum (cisplatin or CDDP), more particularly as sole treatment, will be detrimental to the subject.

In another particular embodiment, the method comprises a step of determining the presence or absence of LIPC in a biological sample of said subject, the presence of LIPC in the biological sample, or an over expression thereof when compared to a reference expression level, being the indication that another molecule, in particular Orlistat®, should preferably be administered to the patient together with a conventional treatment of cancer such as a chemotherapy, in particular a chemotherapy using cis-platinum.

In a further particular embodiment, the method comprises a step of determining the expression level of a compound selected from PDXK, PDXP and aldehyde dehydrogenase 7 family, member A1 (ALDH7A1) in a biological sample of said subject, the non expression thereof or a reduced expression thereof, when compared to a reference expression level, being the indication that a compound selected from vitamin B6, pyridoxal (PL), pyridoxal-5-phosphate (PLP), pyridoxamine (PM), pyridoxamine-5′-phosphate, pyridoxine (PN), pyridoxine-5′-phosphate, L-2-aminoadipate and L-2-aminoadipate 6-semialdehyde, should preferably be administered to the patient together with a conventional treatment of cancer such as a chemotherapy, in particular a chemotherapy using cis-platinum.

Herein disclosed is also a method for screening or identifying a compound suitable for improving the treatment of a cancer (also herein identified as “compensatory molecule”) in a subject having a tumor, said method comprising determining the ability of a test compound to modify the expression in particular of at least one gene selected from the genes identified in Table I, or compensate an abnormal or altered expression thereof.

Further herein disclosed is the use of a compensatory molecule as herein described for treating a cancer or to prepare a pharmaceutical composition for treating a cancer in a subject, identified, by a method as herein described, as resistant to a treatment of cancer, typically to a conventional treatment of cancer. Preferably, the pharmaceutical composition further comprises, as a combined preparation, a drug used in a conventional treatment of cancer, for simultaneous, separate or sequential use in the treatment of said cancer.

Also herein disclosed is a method of assessing the prognosis of a cancer in a subject, the method comprising a step of determining, in a biological sample of said subject, the presence, absence or expression level of a compound selected from vitamin B6, pyridoxal (PL), pyridoxal-5-phosphate (PLP), pyridoxamine (PM), pyridoxamine-5′-phosphate, pyridoxine (PN), pyridoxine-5′-phosphate, L-2-aminoadipate and L-2-aminoadipate 6-semialdehyde, and/or of the expression product of at least one gene selected from the genes identified in Table I, or a derivative product thereof, thereby assessing the prognosis of the cancer in the subject.

Herein described is, in addition, a method of treating cancer comprising the administration to a subject in need thereof, as previously explained, of a compensatory molecule, preferably together with a drug used in a conventional treatment of cancer (as a combined preparation), the molecules being administrable together or separately in a common or unique protocol.

Further herein described is a DNA chip comprising a solid support which carries nucleic acids that are specific to at least two genes, for example three genes, selected from the genes identified in Table I.

Also herein described is a kit for assessing or monitoring the sensitivity of a subject having a tumor to a treatment of cancer, in particular a chemotherapy, or the prognosis of a cancer in a subject, wherein the kit comprises (i) detection means selected from the group consisting of a pair of primers, a probe, at least one antibody specific to the expression product of a gene selected, in particular from the genes identified in Table I, and/or specific to a derivative product thereof, and a DNA chip as herein described, and, optionally, (ii) a leaflet providing the wild-type sequence of the gene and/or the control quantitative expression value corresponding to the expression product of the gene in a control population and/or corresponding to a derivative product thereof.

### LEGENDS TO THE FIGURES

FIG. 1. Genome-wide siRNA screen for the identification of novel CRMs.

(A) siRNA screening setup. CDDP, cisplatin.

(B) Results of the 2^{nd} round of siRNA screen. Each tested siRNA (4×1,000) has been given a score of −1=statistically significant chemosensitizer, 0=cytotoxic or antiproliferative or non-affecting CDDP-induced cell death, +1=statistically significant cytoprotector. mRNA scores were calculated by summing the scores of the corresponding siRNAs. 85 transcripts with a score of −4, −3, +3 or +4 were retained for further analysis. See also Table 1.

(C) Effects of the depletion of druggable hits from the 2^{nd} round of siRNA screen on the response of A549 cells to CDDP. Δ represents the difference between the residual proliferation (upon the administration of CDDP) in siRNA-transfected cells and the residual proliferation in cells transfected with a control, non-targeting siRNA (siUNR). See also Supplementary Materials and Methods. Mean±SEM (n=3). All results are statistically significant (Student's t test, p<0.05), as compared to control conditions (UNR).

(D,E) Cytofluorometric assessment of cell death induced by 25 or 75 μM CDDP in A549 cells transfected with siRNAs targeting the indicated proteins (D) or co-treated with the indicated molecules. PI⁺ DiOC₃(6)^{low}=dead cells; PI⁻ DiOC₃(6)^{low}=dying cells. Mean±SEM (n=3). *=p<0.05 (Student's t test), as compared to untreated siUNR-transfected (D) or untransfected cells (E). #=p<0.05 (Student's t test), as compared to siUNR-transfected (D) or untransfected (E) cells treated with the same concentration of CDDP. See also FIG. 8A,B and Table 2.

FIG. 2. Genetic and functional signatures of A549 cells responding to CDDP, C₂-CER and CdCl₂.

(A) Unsupervised hierarchical clustering of the transcriptional modifications induced in A549 cells by the administration of cisplatin (CDDP), C₂-ceramide (C₂-CER) and cadmium chloride (CdCl₂) for 12 or 24 h. FC=fold change, as compared to control conditions (untreated cells).

(B) Graphical representation of the absolute number of transcripts significantly up-(FC>+1.2) or downregulated (FC<−1.2) in the experimental conditions described in (A). Venn diagrams depict the overlap among these transcriptional signatures.

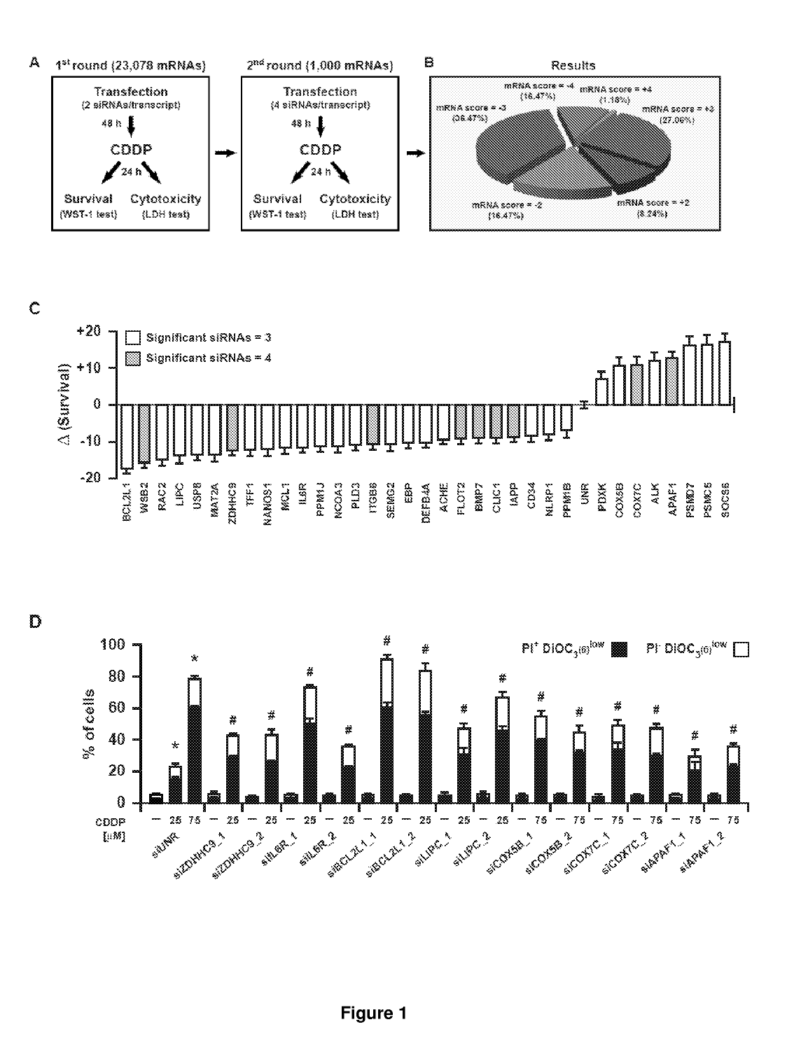

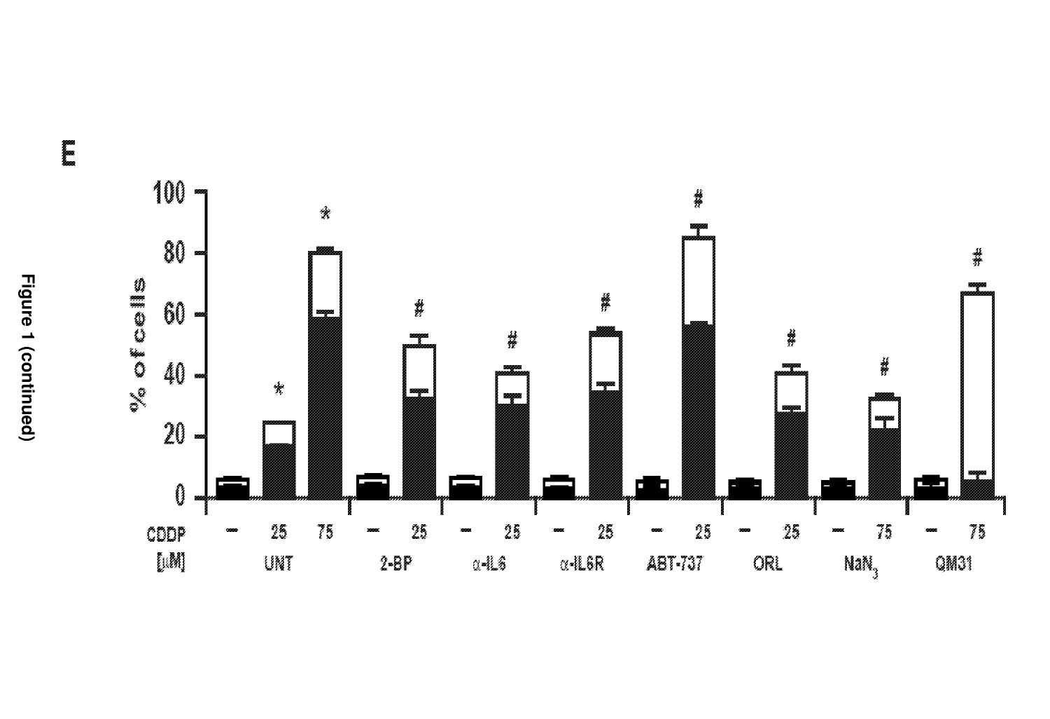

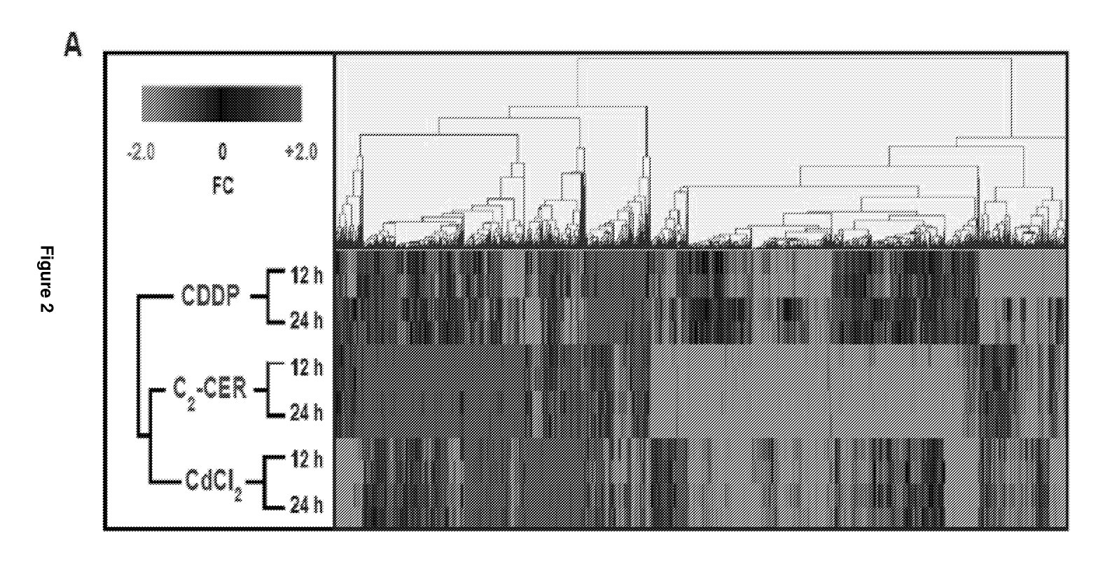

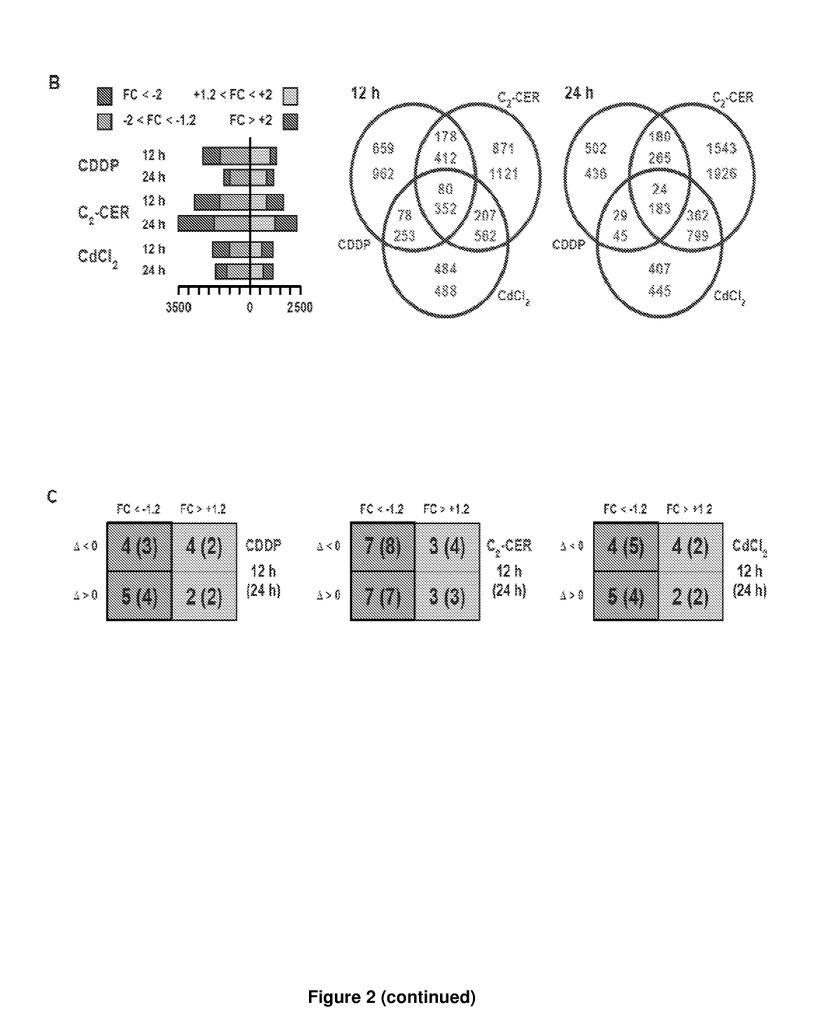

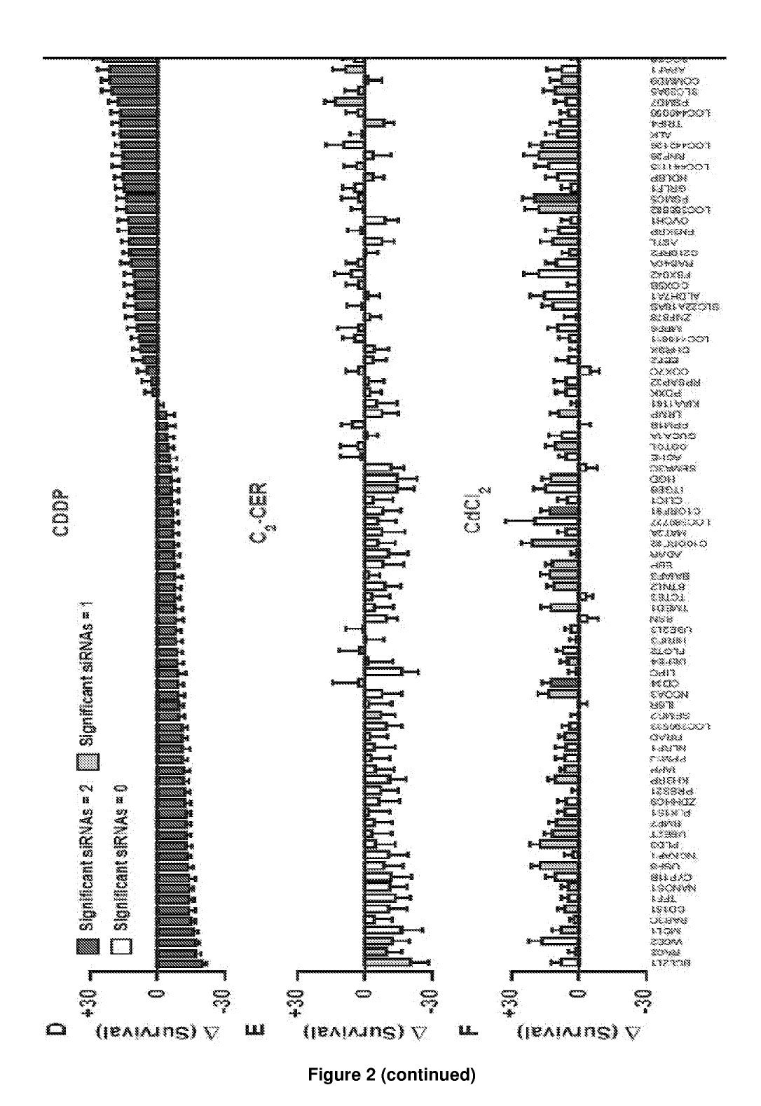

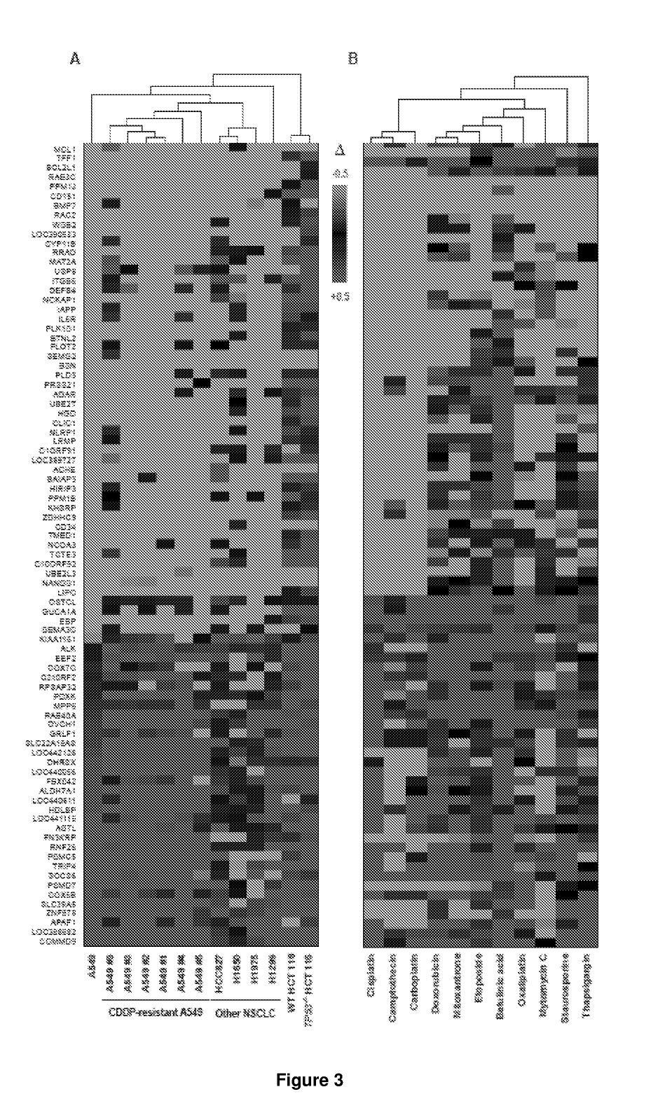

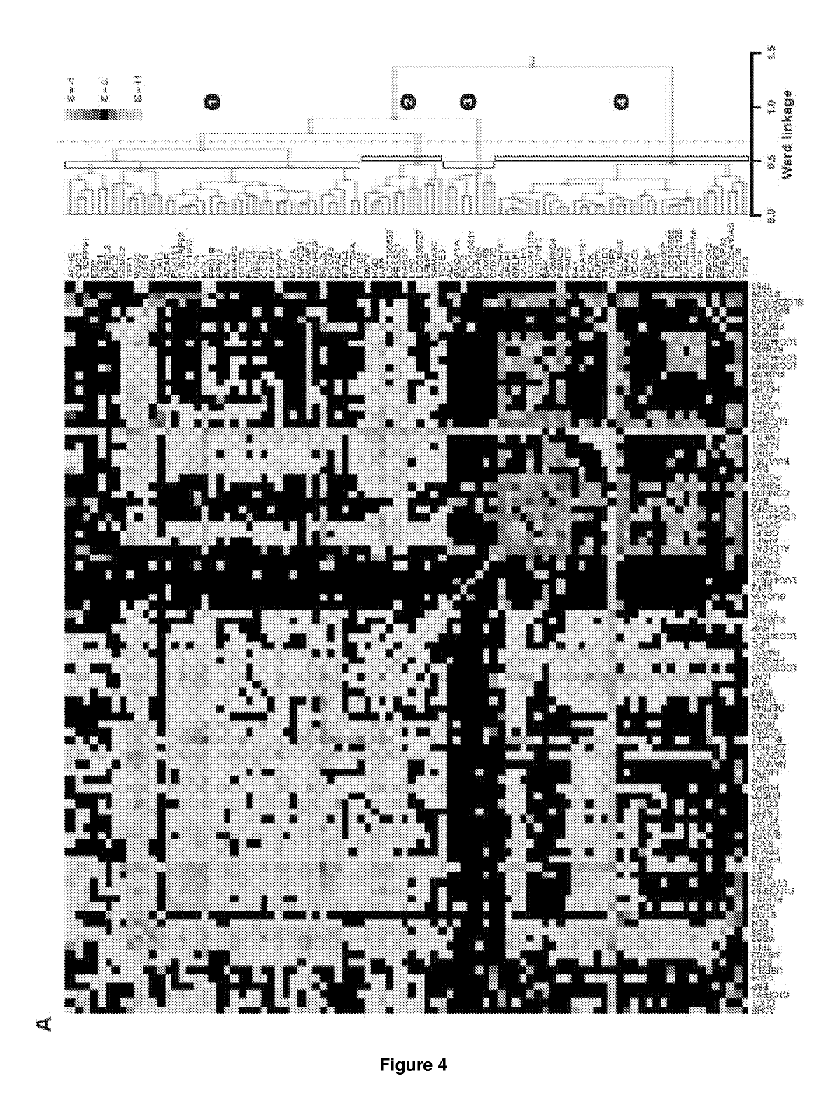

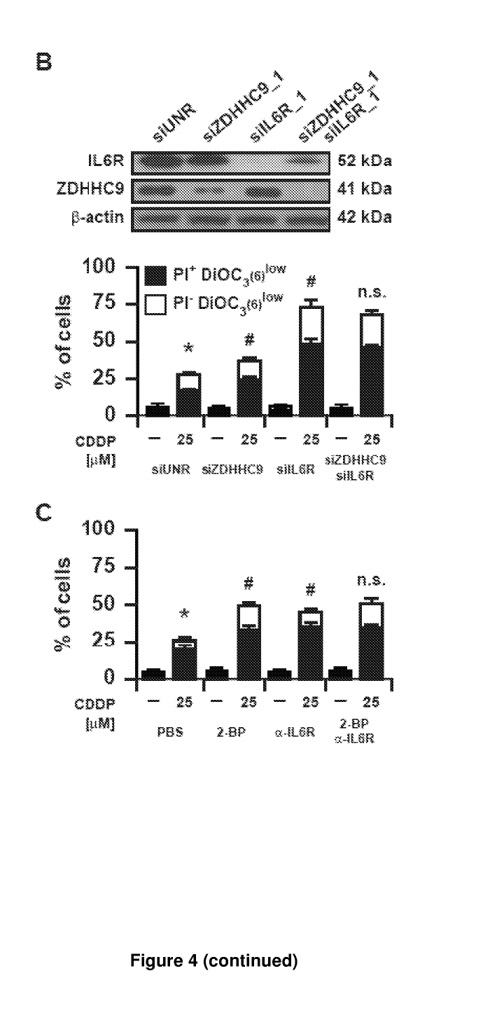

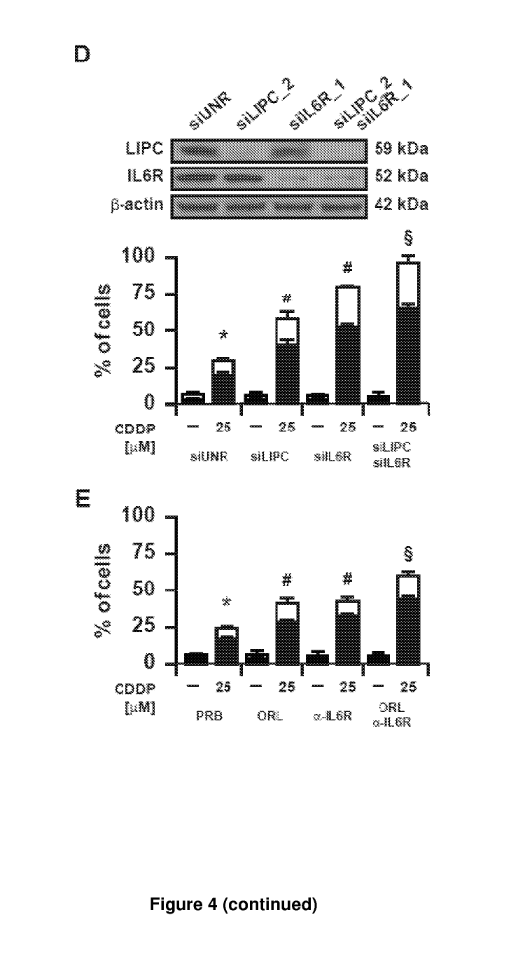

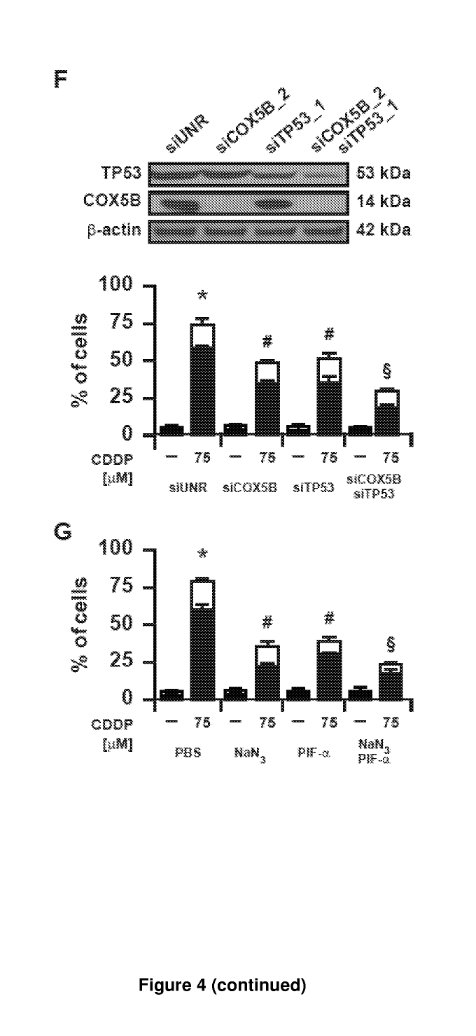

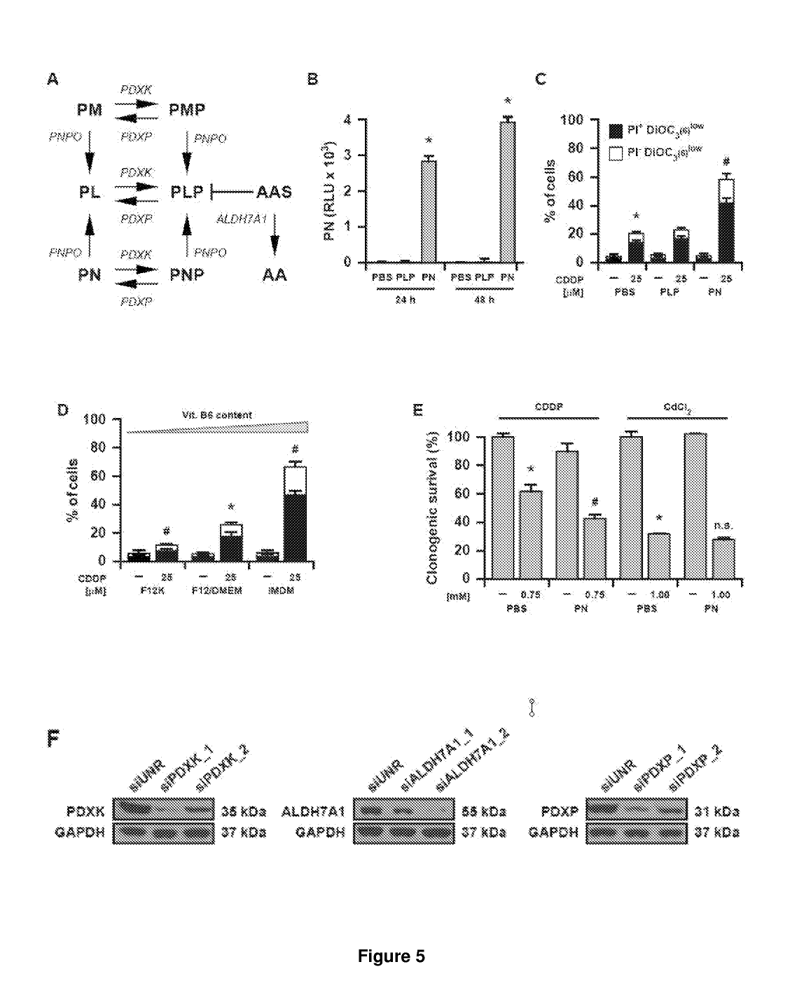

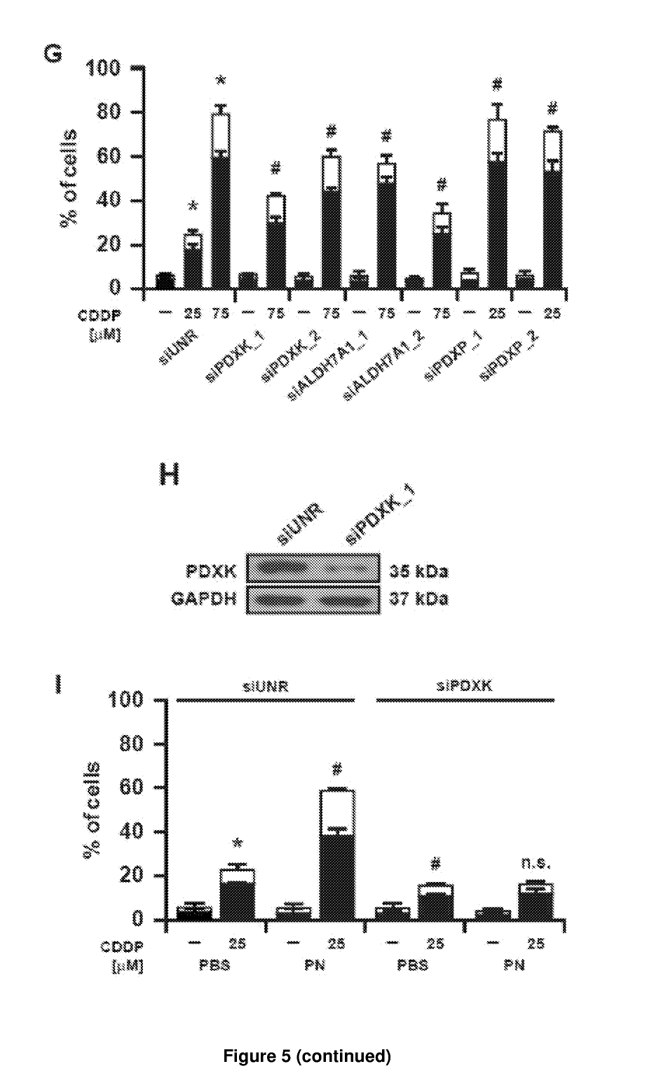

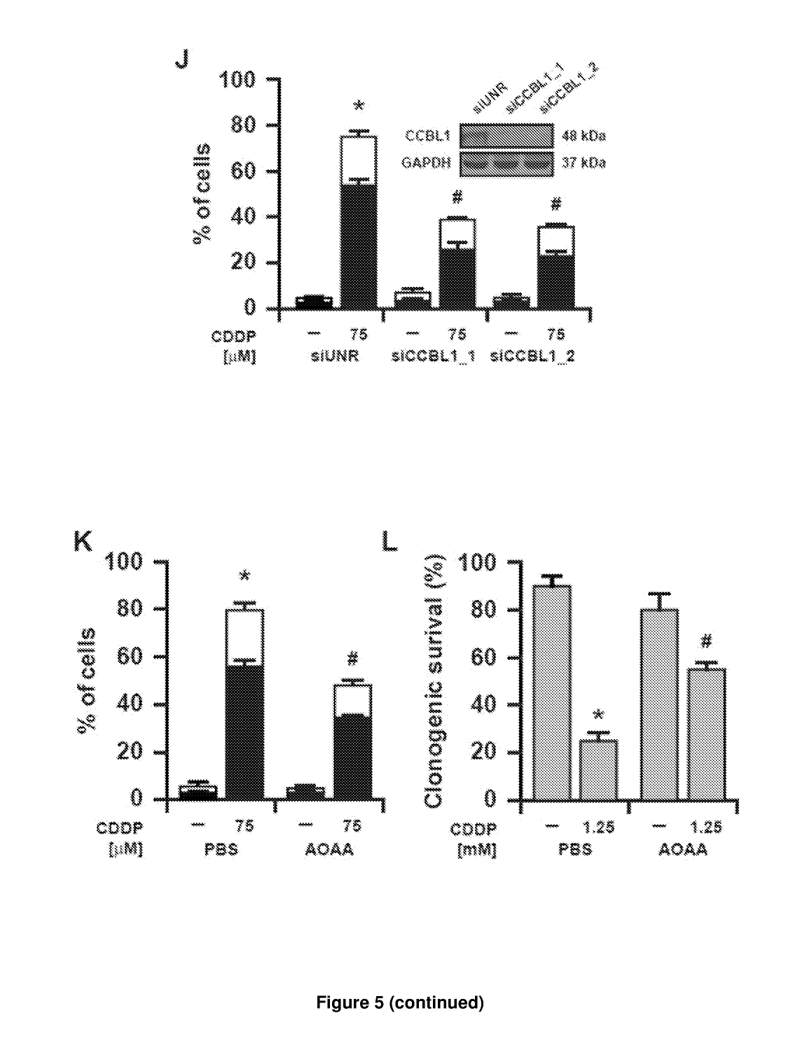

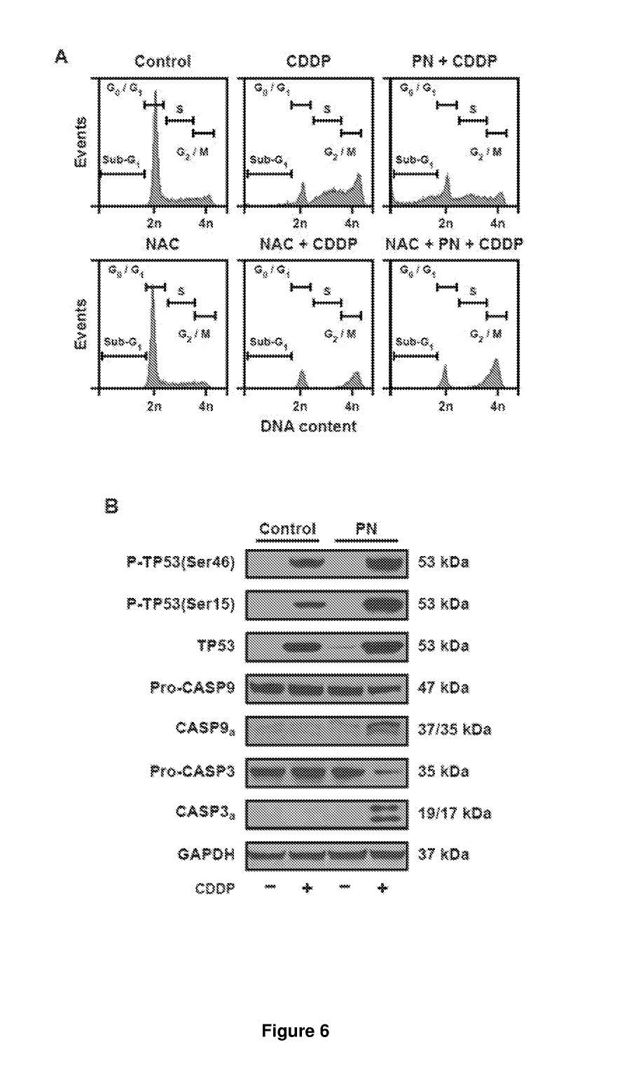

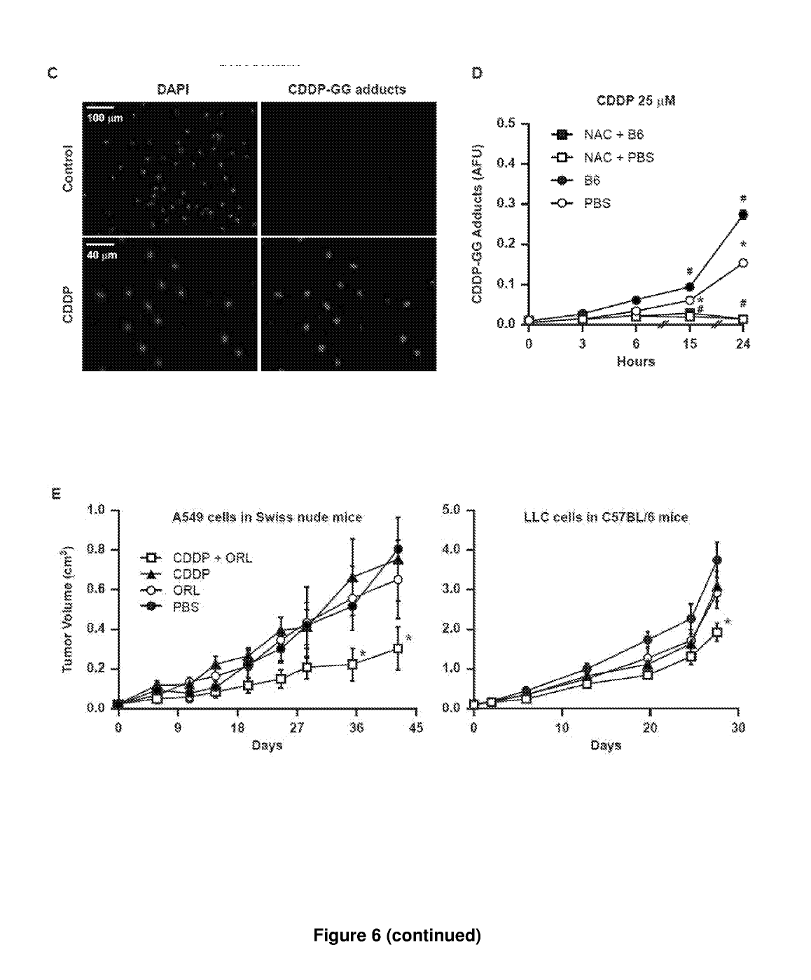

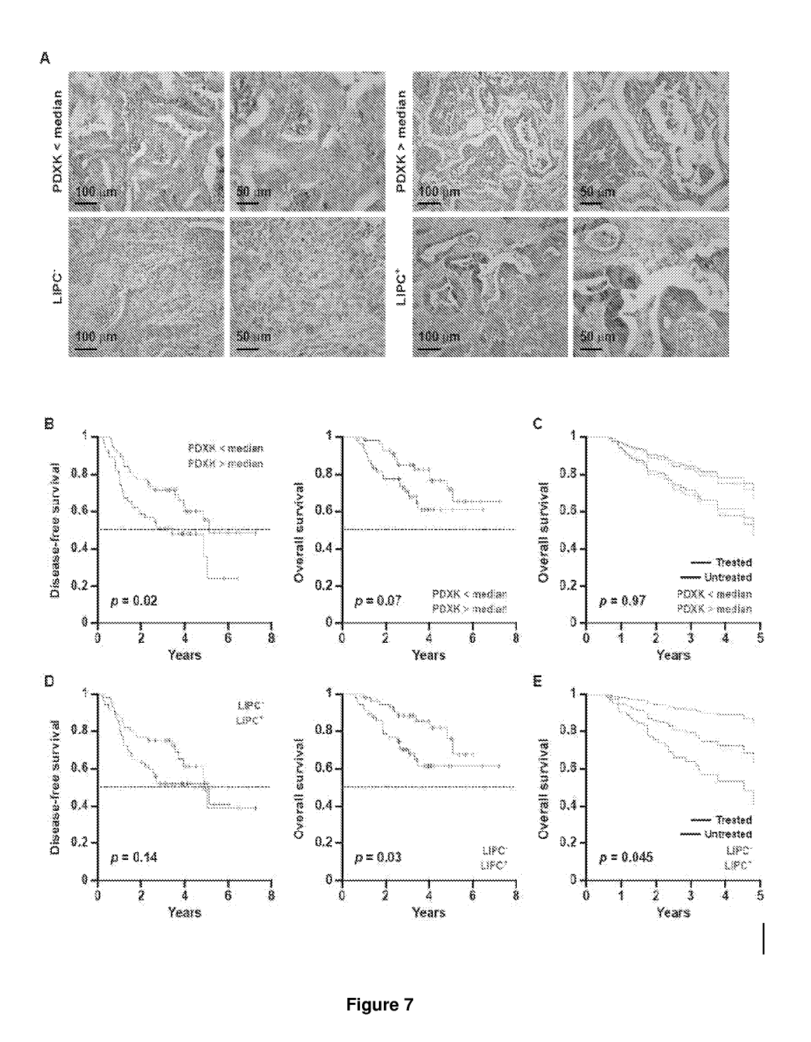

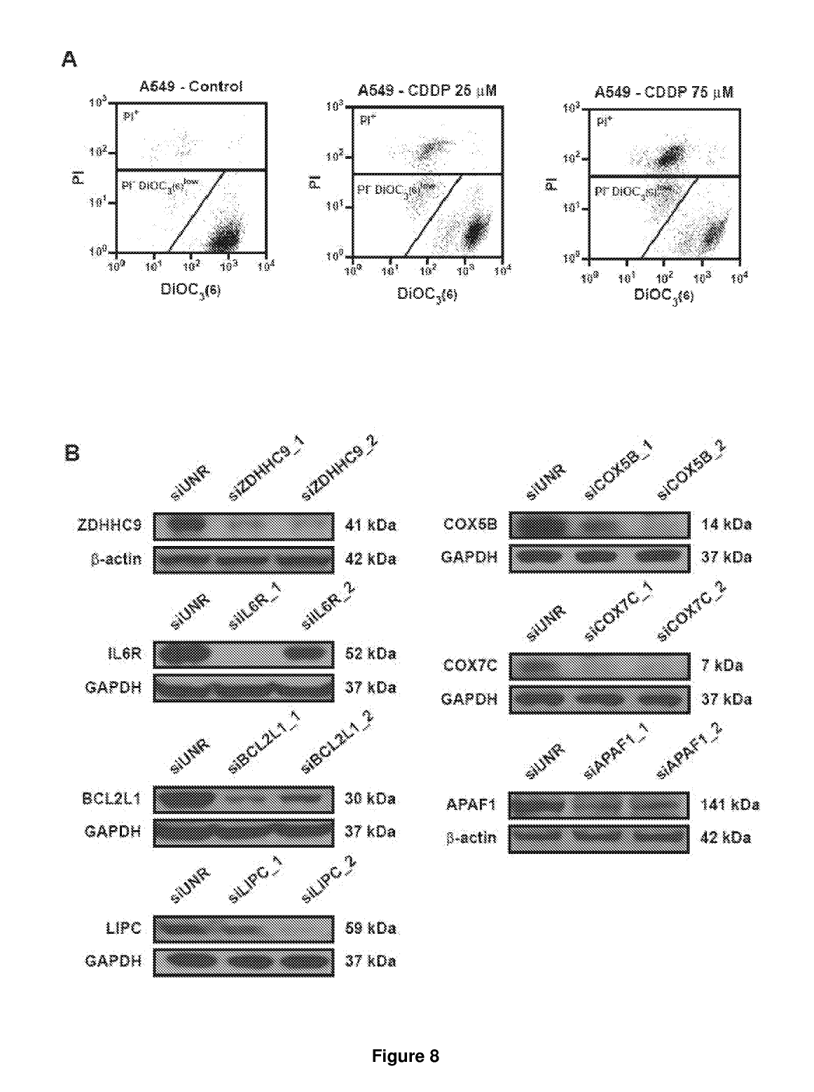

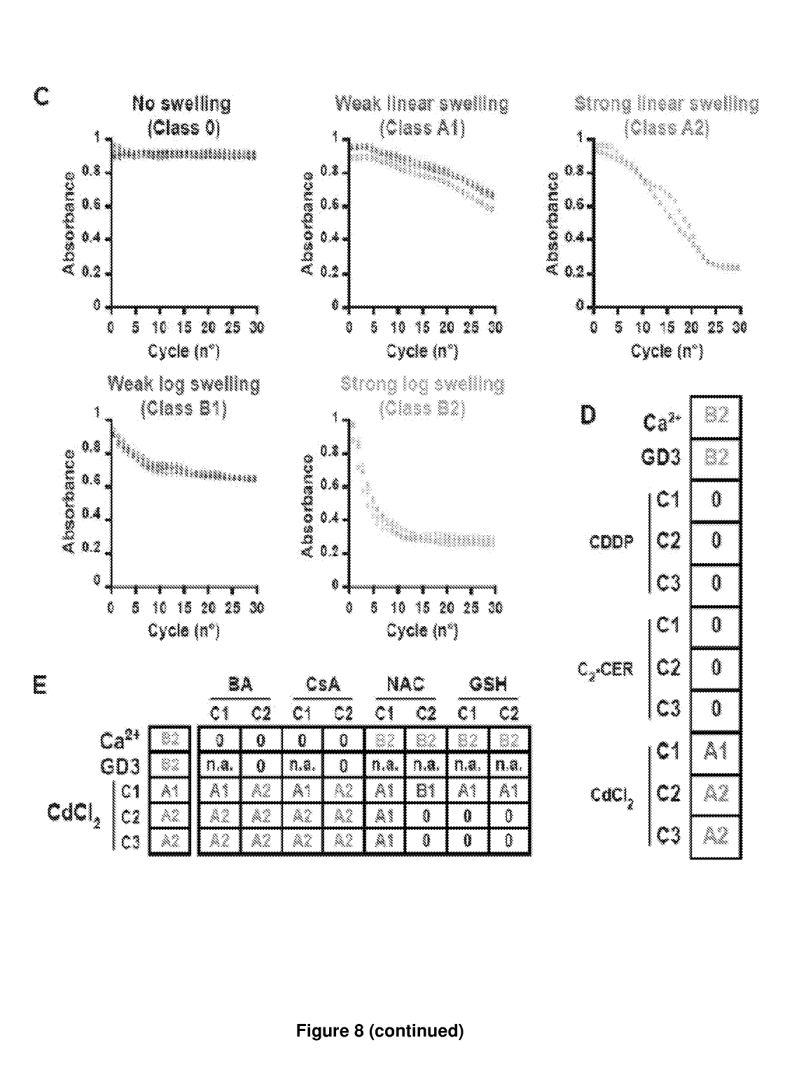

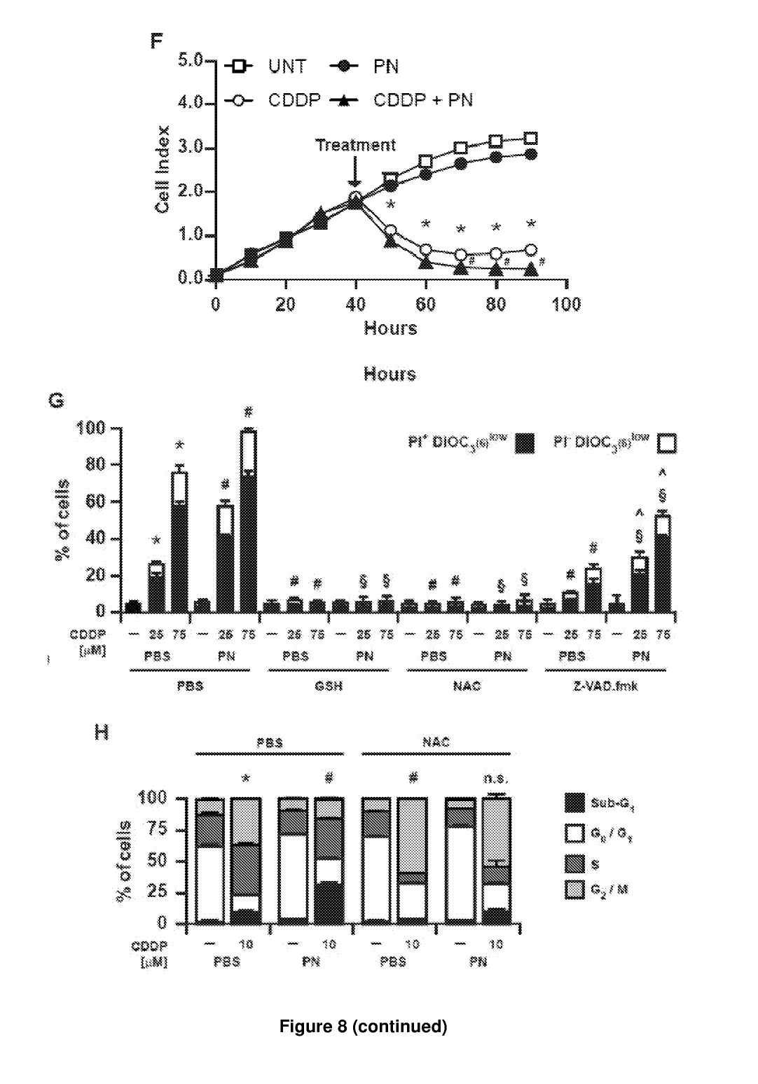

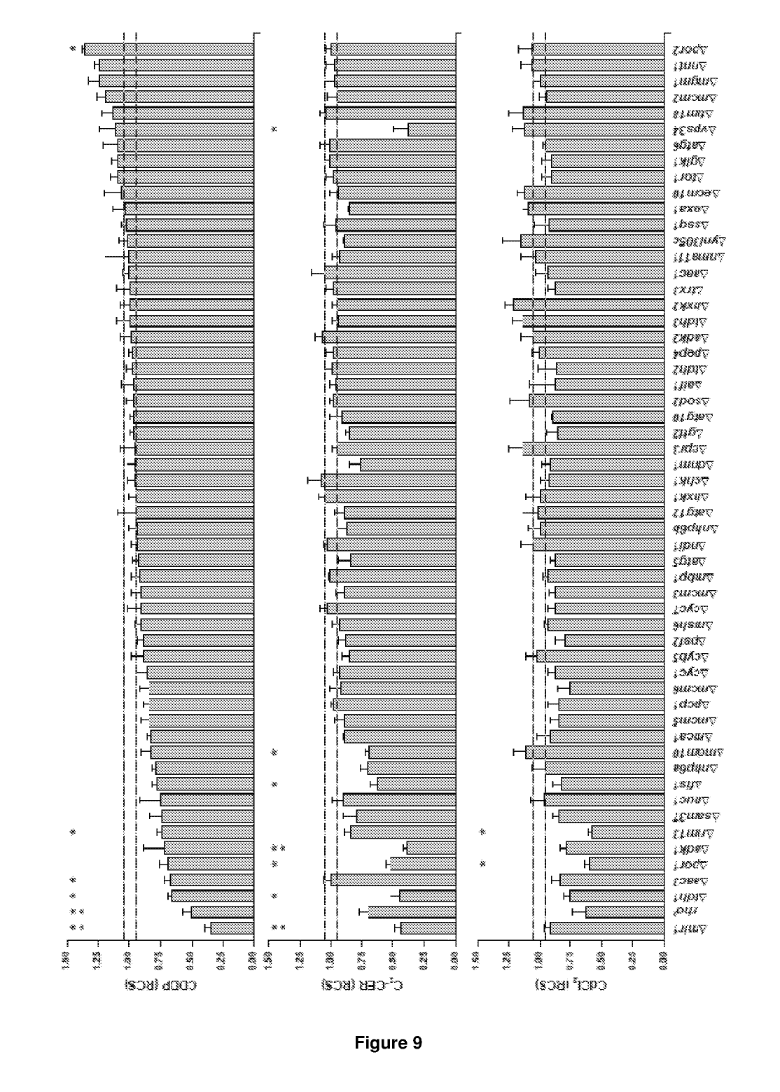

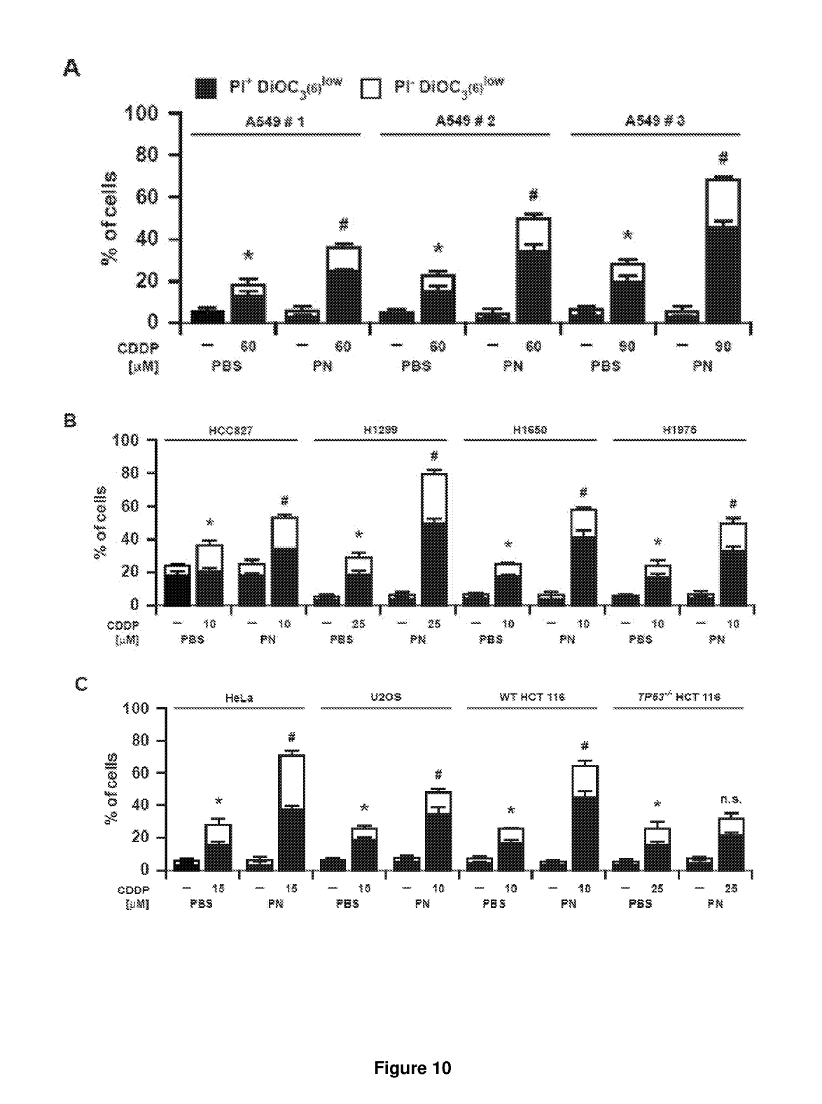

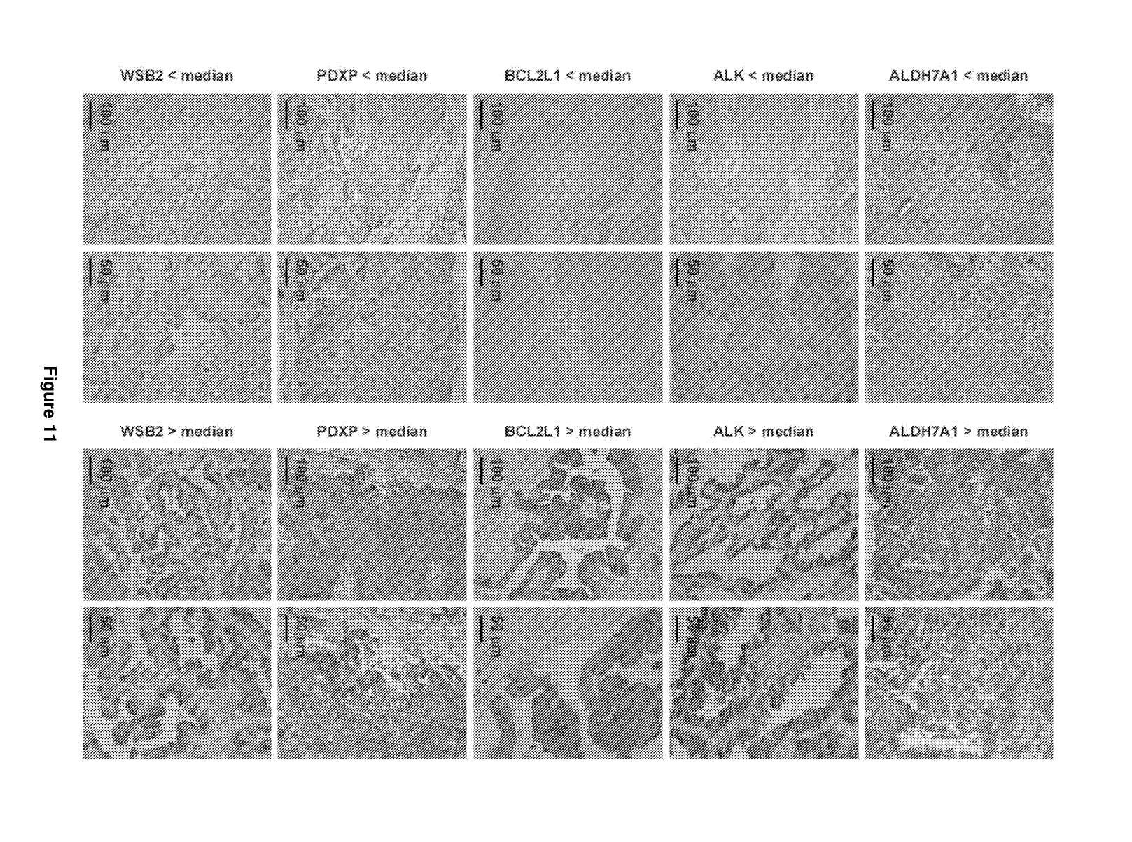

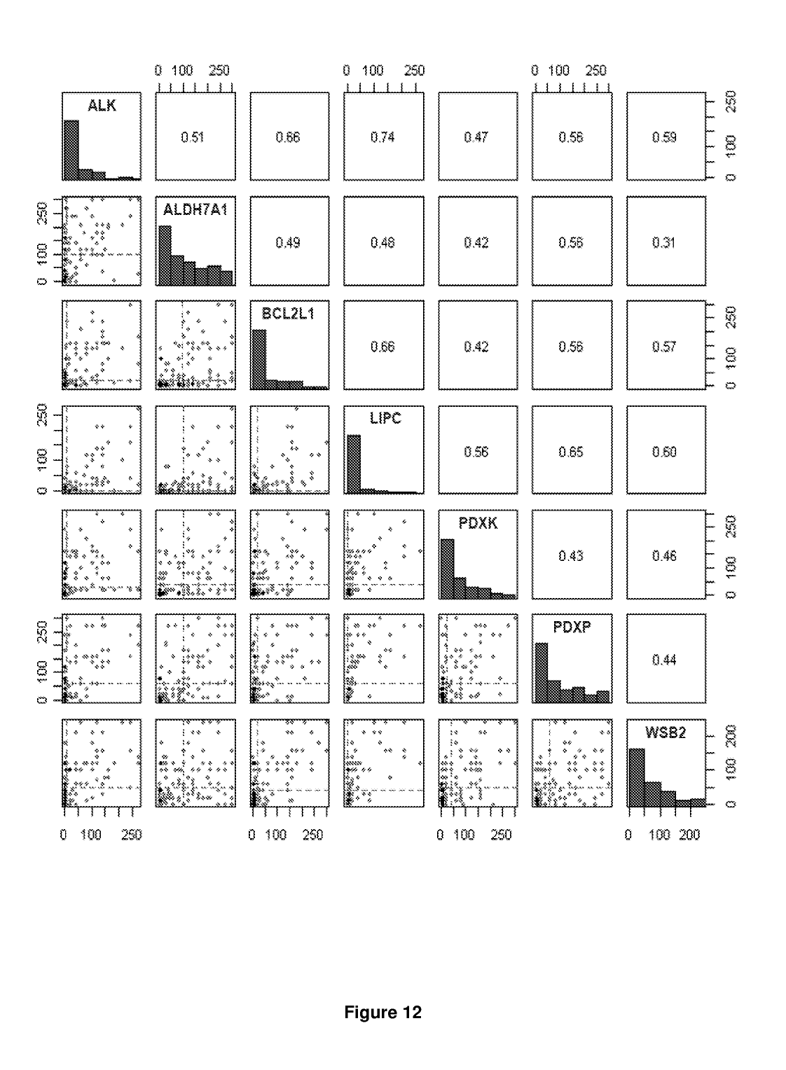

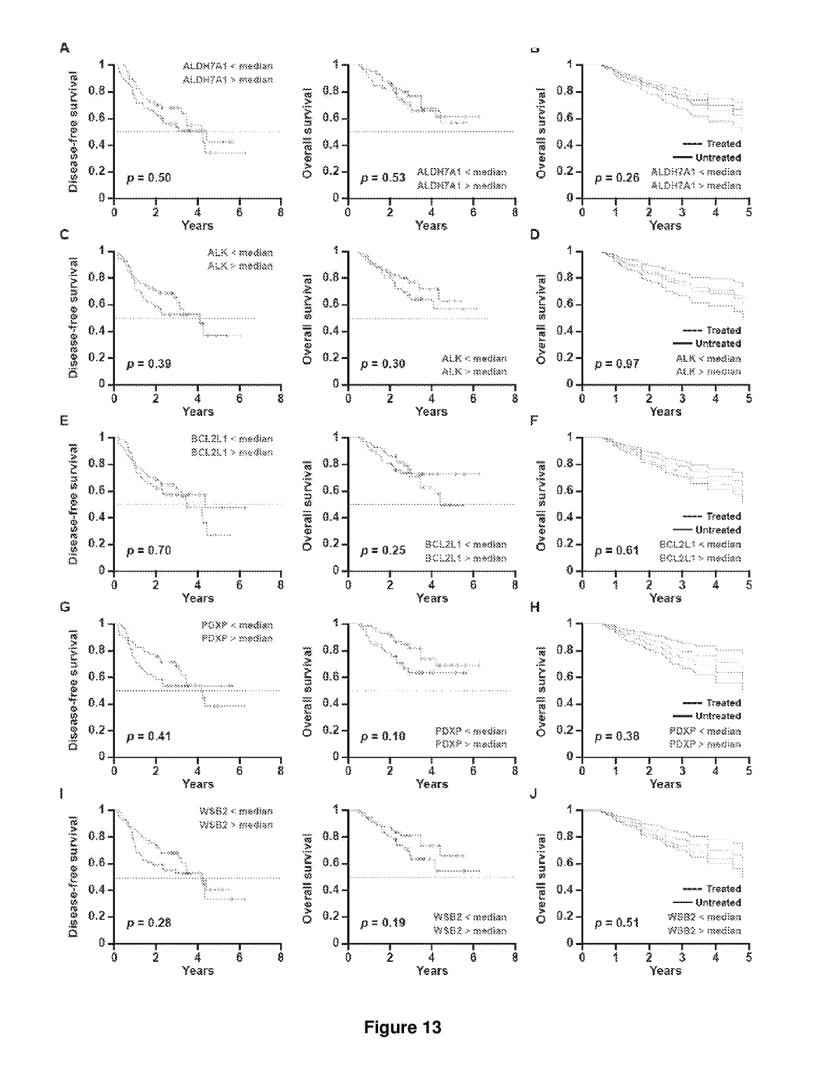

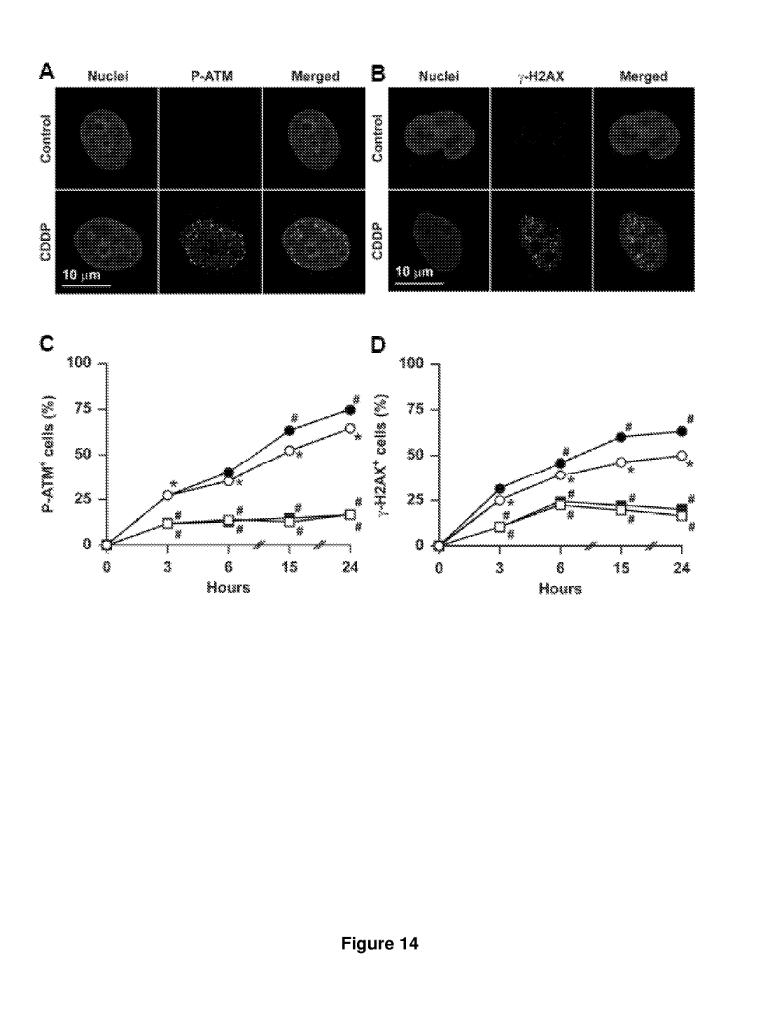

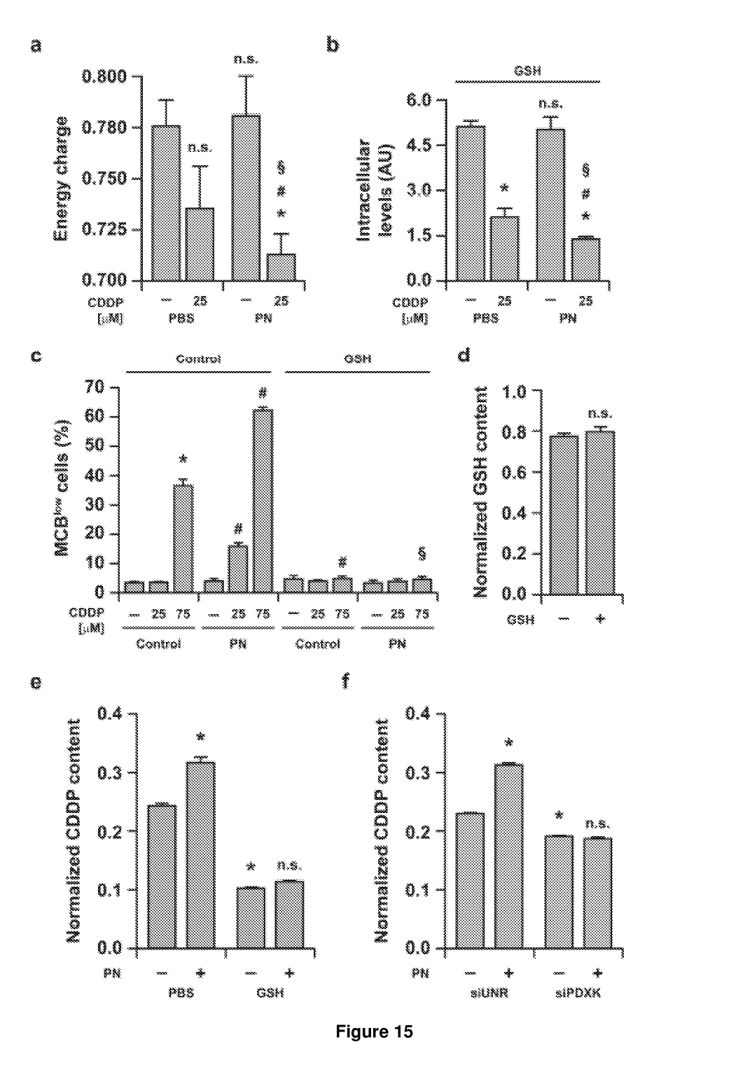

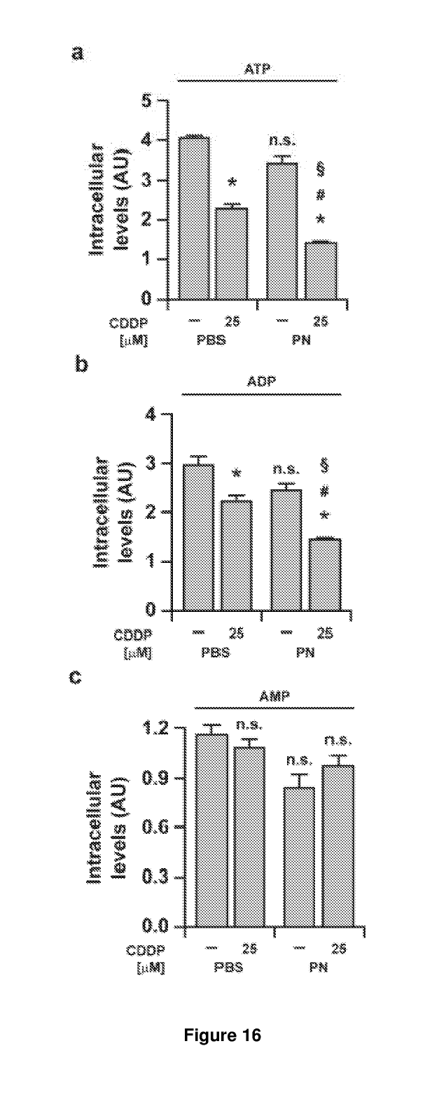

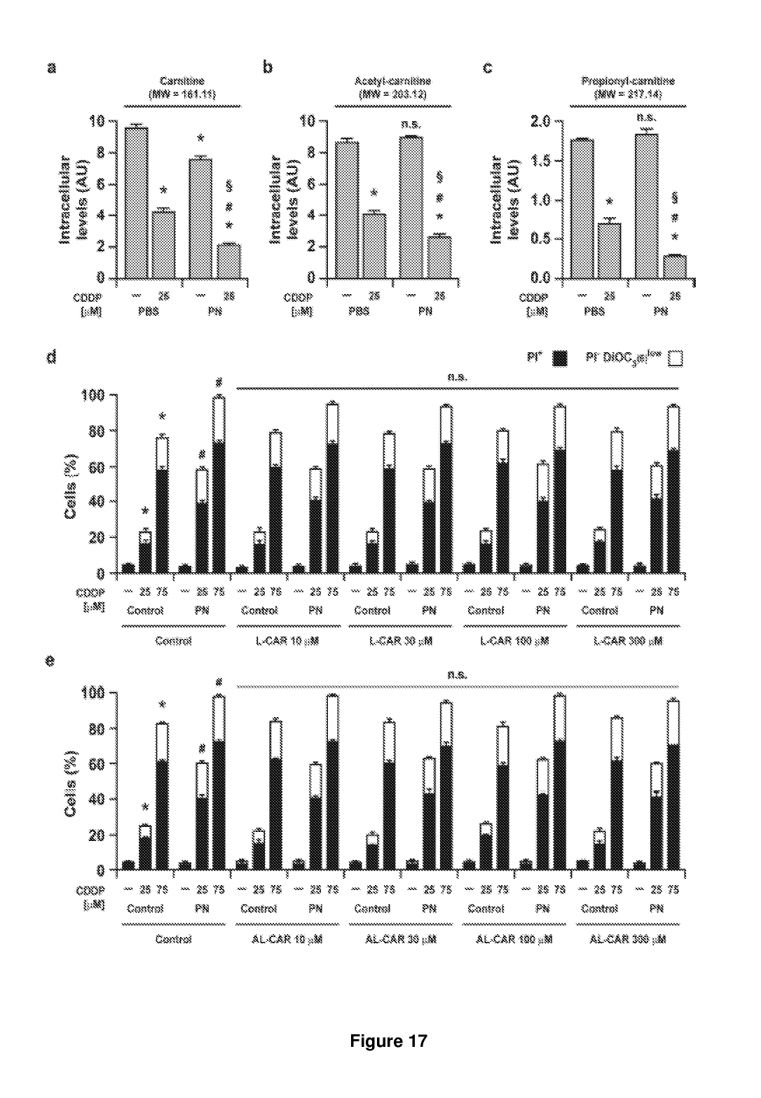

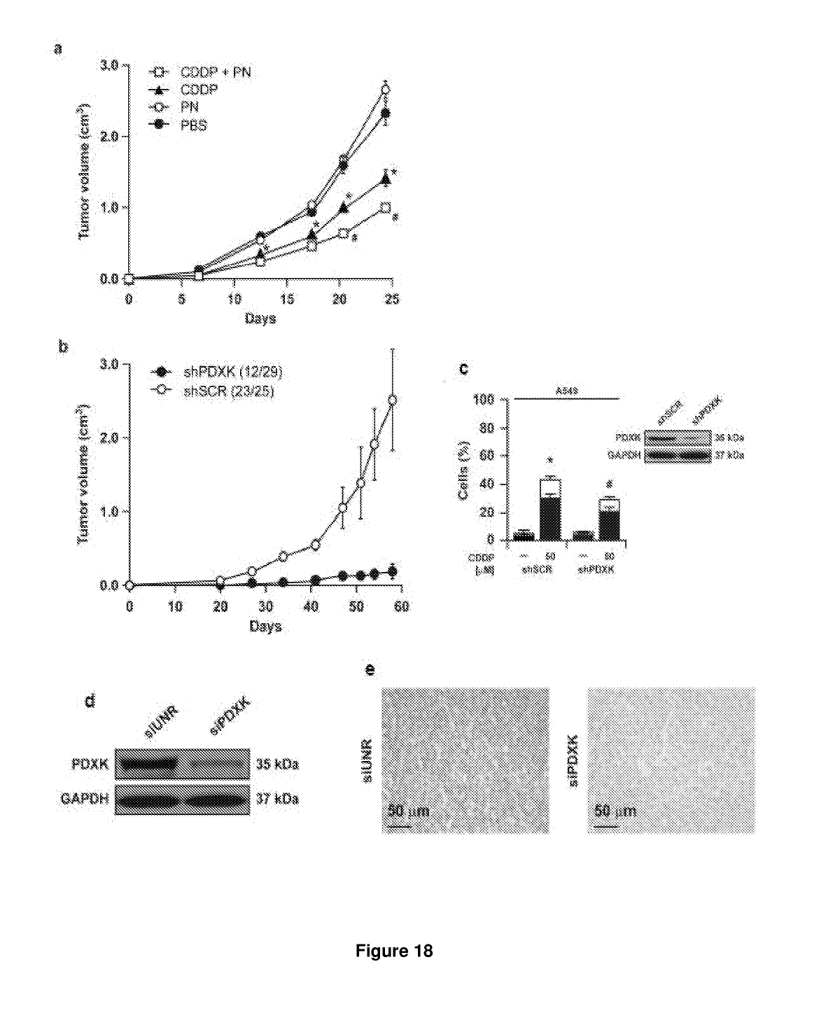

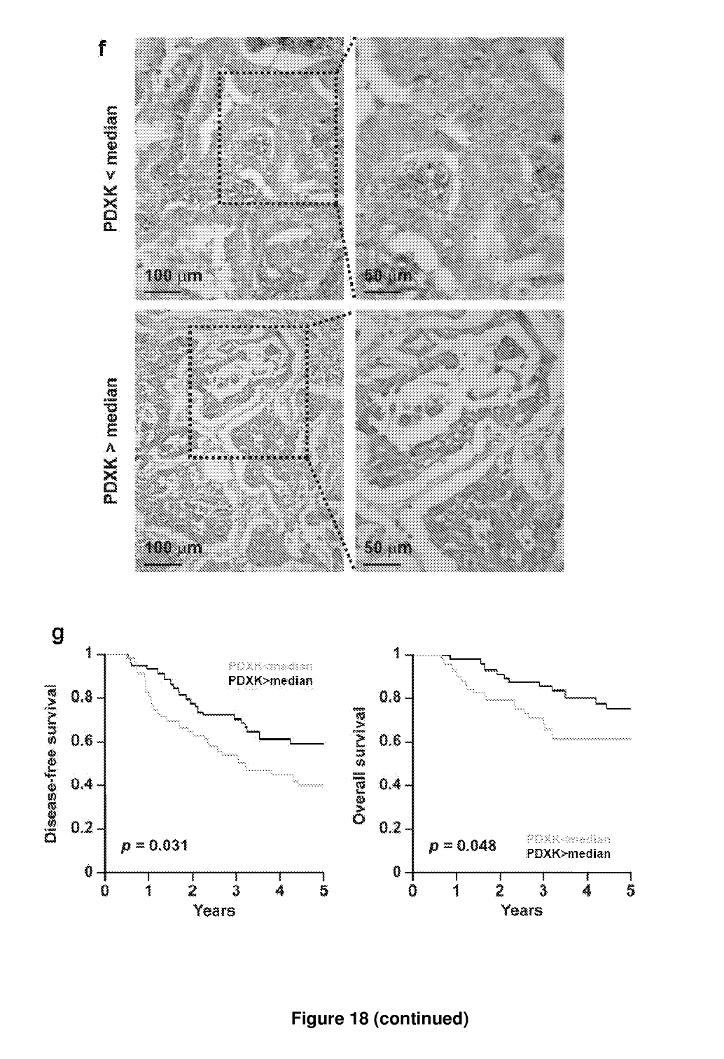

(C) Overlap between the 85 hits from the 2^{nd} round of siRNA screen and the transcriptional signatures induced by CDDP, C₂-CER and CdCl₂. Δ is defined as in FIG. 1C (See also Materials and Methods of example 1). See also Table 3.

(D-F) Effects of the depletion of the 85 hits from the 2^{nd} round of siRNA screen on the response of A549 cells to CDDP (D), C₂-CER (E) and CdCl₂ (F). Δ is defined as in FIG. 1C (See also Materials and Methods of example 1). Mean±SEM (n=3). Significance (Student's t test, p<0.05, as compared to siUNR-transfected cells) is color-coded: dark grey=2 significant siRNAs; light grey=1 significant siRNA; white=non significant.

FIG. 3. Functional characterization of CRMs in multiple cancer cell lines and in A549 cells challenged with CDDP-related and -unrelated cell death inducers.

(A) Unsupervised hierarchical clustering of the effects of the 85 hits from the 2^{nd} round of siRNA screen on the response to cisplatin (CDDP) of wild type (WT) human non-small cell lung cancer (NSCLC) A549 cells (control condition), of six distinct CDDP-resistant A549 clones (A549 #1-6), of four NSCLC cell lines other than A549 (i.e., HCC827, H1299, H1650 and H1975 cells) as well as of WT and TP53^{-/-} colon carcinoma HCT 116 cells. Δ is defined as in FIG. 1C (See also Materials and Methods of example 1). See also Table 4.

(B) Unsupervised hierarchical clustering of the effects of the 85 hits from the 2^{nd} round of siRNA screen on the response of human NSCLC A549 cells to CDDP (control condition) and to betulinic acid, carboplatin, camptothecin, doxorubicine, etoposide, mitoxantrone, mytomycin C, oxaliplatin, staurosporine, thapsigargin. Δ is defined as in FIG. 1C (See also Materials and Methods of example 1). See also Table 4.

FIG. 4. Epistatic clustering and validation of the interactions between CRMs.

(A) Epistatic clustering of the effects of the co-transfection of each of the 85 hits from the 2^{nd} round with each other on the response of A549 cells to cisplatin (CDDP). ε (varying from −1 to +1) indicates the degree of epistatic interaction (See also Materials and Methods of example 1). This analysis allows for the identification of 4 clusters of CDDP response modifiers. See also Table 5.

(B-G) Cytofluorometric validation of the epistatic clustering depicted in A upon transfection of A549 cells with the indicated siRNAs (alone or in couples) (B, D, F) or treatment with the indicated agents (alone or in combination) (C, E, G) followed by CDDP administration. PI⁺ DiOC₃(6)^{low}=dead cells; PI⁻ DiOC₃(6)^{low}=dying cells. Mean±SEM (n=3). *=p<0.05 (Student's t test), as compared to untreated siUNR-transfected (B, D, F) or untransfected cells (C, E, G). #=p<0.05 (Student's t test), as compared to siUNR-transfected (B, D, F) or untransfected (C, E, G) cells treated with the same concentration of CDDP. §=p<0.05 (Student's t test), as compared to cells transfected with either siRNA alone (D,F) or cells treated with either pharmacological modulator alone (E,G) and the same concentration of CDDP. n.s.=non significant (Student's t test), as compared to cells transfected with either siRNA alone (B) or cells treated with either pharmacological modulator alone (C) and the same concentration of CDDP. Immunoblots depict the efficiency of siRNA-mediated target downregulation. β-actin or glyceraldehyde-3-phosphate dehydrogenase (GAPDH) levels were assessed to monitor equal loading of lanes.

FIG. 5. Influence of the vitamin B6 metabolism in the response to CDDP of NSCLC cells and Saccharomyces cerevisiae.

(A) Vitamin B6 metabolism in humans. AA, L-2-aminoadipate; AAS, L-2-aminoadipate 6-semialdehyde; ALDH7A1, aldehyde dehydrogenase 7 family, member A1; PDXK, pyridoxal kinase; PDXP, pyridozxal phosphatase; PNPO, pyridoxamine 5′-phosphate oxidase; PL, pyridoxal; PLP, pyridoxal-5-phosphate; PM, pyridoxamine; PMP, pyridoxamine-5′-phosphate; PN, pyridoxine; PNP, pyridoxine-5′-phosphate.

(B) Quantification of intracellular PN upon acute administration of PLP and PN to A549 cells for 24 or 48 h. *=p<0.001 (Student's t test), as compared to PBS-treated cells.

(C, D) Cytofluorometric assessment of cell death induced by 25 μM cisplatin (CDDP) in A549 cells co-treated with the indicated vitamin B6 vitamers for 48 h (C) or previously cultured for 1 month in media containing different amounts of vitamin B6 vitamers (D). PI⁺ DiOC₃(6)^{low}=dead cells; PI⁻ DiOC₃(6)^{low}=dying cells. Mean±SEM (n=3). *=p<0.05 (Student's t test), as compared to untreated cells cultured in standard conditions (DMEM/F12 medium). #=p<0.05 (Student's t test), as compared to cells maintained in standard conditions (DMEM/F12 medium) and treated with the same concentration of CDDP. See also FIG. 8F.

(E) Clonogenic survival of Saccharomyces cerevisiae cells treated with the indicated concentration of CDDP or cadmium dichloride (CdCl₂) in the absence or in the presence of PN. Mean±SEM, normalized to control conditions (n=3). *=p<0.05 (Student's t test), as compared to untreated cells. #=p<0.05 (Student's t test), as compared to cells incubated with the same concentration of CDDP. n.s.=non significant (Student's t test), as compared to cells incubated with the same concentration of CdCl₂.

(F, G) Cytofluorometric assessment of cell death induced by 25 or 75 μM CDDP in A549 cells transfected with siRNAs targeting the indicated vitamin B6-relevant enzymes. Immunoblots depict the efficiency of siRNA-mediated target downregulation. Glyceraldehyde-3-phosphate dehydrogenase (GAPDH) levels were monitored to ensure equal loading of lanes (F). PI⁺ DiOC₃(6)^{low}=dead cells; PI⁻ DiOC₃(6)^{low}=dying cells. Mean±SEM (n=3). *=p<0.05 (Student's t test), as compared to untreated siUNR-transfected cells. #=p<0.05 (Student's t test), as compared to siUNR-transfected cells treated with the same concentration of CDDP (G).

(H, I) Combined effects of PDXK depletion and PN administration on CDDP-induced cell death. Immunoblots depict the efficiency of siRNA-mediated target downregulation. GAPDH levels were assessed to monitor equal lane loading (H). PI⁺ DiOC₃(6)^{low}=dead cells; PI⁻ DiOC₃(6)^{low}=dying cells. Mean±SEM (n=3). *=p<0.05 (Student's t test), as compared to untreated siUNR-transfected cells. #=p<0.05 (Student's t test), as compared to siUNR-transfected cells treated with the same concentration of CDDP. n.s.=non significant (Student's t test), as compared to siPDXK-transfected cells treated with the same concentration of CDDP (I).

(J, K) Cytofluorometric quantification of cell death induced by 75 μM CDDP in A549 cells transfected with siRNAs targeting cysteine conjugate-β lyase (CCBL1) (J) or co-treated with the CCBL1 inhibitor aminooxyacetic acid (AOAA) (K). The insert in (J) shows the efficiency of siRNA-mediated CCBL1 depletion. GAPDH abundance was monitored to ensure equal loading of lanes. PI⁺ DiOC₃(6)^{low}=dead cells; PI⁻ DiOC₃(6)^{low}=dying cells. Mean±SEM (n=3). *=p<0.05 (Student's t test), as compared to untreated siUNR-transfected (J) or untransfected (K) cells. #=p<0.05 (Student's t test), as compared to siUNR-transfected (J) or untransfected (K) cells treated with the same concentration of CDDP.

(L) Clonogenic survival of yeast cells treated with the indicated concentration of CDDP alone or in the presence of AOAA. Mean±SEM (n=3). *=p<0.05 (Student's t test), as compared to untreated cells. #=p<0.05 (Student's t test), as compared to cells incubated with the same concentration of CDDP.

FIG. 6. Functional characterization of chemosensitization mediated by PN and LIPC inhibition in vitro and in vivo.

(A) Cell cycle distributions of A549 cells treated with 10 μM cisplatin (CDDP) alone or in combination with pyridoxine (PN) and/or N-acetylcysteine (NAC). See also FIG. 8G, H.

(B) Immunoblotting-based assessment of the activation of the intrinsic apoptosis pathway in A549 cells treated with 25 μM CDDP alone or in combination with PN. CASP, caspase. Glyceraldehyde-3-phosphate dehydrogenase (GAPDH) levels were monitored to ensure balanced lane loading.

(C, D) Immunofluorescence microscopic evaluation of CDDP-DNA adducts in A549 cells responding to 25 μM CDDP alone or combined with PN. Immunofluorescence microphotographs of untreated versus CDDP-treated cells. White bars indicate picture scale (C). Quantitative kinetic data as obtained upon automatic image analysis. Mean±SEM (n=3). *=p<0.05 (Student's t test), as compared to untreated cells. #=p<0.05 (Student's t test), as compared to cells treated with CDDP only (D).

(E) In vivo growth of human A549 cells xenografted into immunodeficient Swiss nude mice (left panel) and murine Lewis lung carcinoma (LLC) cells xenografted into immunocompetent C57BL/6 mice (right panel) upon treatment with intraperitoneal CDDP alone or in combination with orlistat. Mean±SEM (n=4-6 mice/group). *=p<0.05 (two-way ANOVA), as compared to PBS-treated mice. See also Materials and Methods of example 1.

FIG. 7. Prognostic and predictive value of PDXK and LIPC status in lung cancer patients.

(A) Immunohistochemical detection of pyridoxal kinase (PDXK) and hepatic lipase (LIPC) levels in biopsies from lung cancer patients. Bars indicate picture scale. See also FIG. 11-13.

(B, D) Kaplan-Meier curves for disease-free survival and overall upon stratification of patients according to the median of expression of PDXK (B) or LIPC (D). p values (log-rank test) are depicted.

(C, E) Estimated survival curves for lung cancer patients stratified according to the median of PDXK (C) or LIPC (E) expression, based on a Cox proportional hazard model with chemotherapy marker interaction. The p value of the interaction is depicted.

FIG. 8. Cytofluorometric quantification of cell death (A, G), immunoblotting-based assessment of the efficiency of siRNA (B), response of isolated mitochondria (C-E), detachment from the substrate of A549 cells treated with 50 μM CDDP (F), cell cycle distributions of A549 cells treated with 10 μM CDDP(H)

(A) Cytofluorometric quantification of cell death induced by 25 or 75 μM cisplatin (CDDP) in A549 cells. PI⁺ events=dead cells; PI⁻ DiOC₃(6)^{low} events=dying cells. Quantitative results are reported in FIG. 1D, E.

(B) Immunoblotting-based assessment of the efficiency of siRNA-mediated target downregulation. β-actin or glyceraldehyde-3-phosphate dehydrogenase (GAPDH) levels were assessed to monitor equal loading of lanes.

(C-E) Response of isolated mitochondria to calcium ions (Ca²⁺), GD3 ganglioside (both employed as positive control conditions), CDDP, C2-ceramide (C₂-CER) and cadmium dichloride (CdCl₂). Large amplitude swelling categories based on intensity and kinetic of absorbance decrease (C). Direct mitochondrion-permeabilizing effects of the indicated molecules (D). Response of Ca²⁺-, GD3- and CdCl₂-mediated swelling to known swelling inhibitors such as bongkrekic acid (BA), cyclosporine A (CsA), N-acetylcysteine (NAC) and non-oxidized glutathione (GSH). n.a.=not assessed (E).

(F) Detachment from the substrate of A549 cells treated with 50 μM CDDP alone or in combination with PN. Cell attachment was quantified as a function of the impedance of special cell culture plates integrated with microelectrodes (See also Materials and Methods of example 1). Mean±SEM (n=3). *=p<0.05 (Student's t test), as compared to untreated siUNR-transfected cells. #=p<0.05 (Student's t test), as compared to siUNR-transfected cells treated with the same concentration of CDDP.

(G) Cytofluorometric quantification of A549 cell death induced by 25 or 75 μM CDDP alone or in combination with GSH, NAC or the pan-caspase inhibitor Z-VAD-fmk. PI⁺ DiOC₃(6)^{low}=dead cells; PI⁻ DiOC₃(6)^{low}=dying cells. Mean±SEM (n=3). *=p<0.05 (Student's t test), as compared to untreated cells. #=p<0.05 (Student's t test), as compared to cells treated with the same concentration of CDDP. §=p<0.05 (Student's t test), as compared to cells treated with the same concentration of CDDP and PN. ̂=p<0.05 (Student's t test), as compared to cells treated with the same concentration of CDDP and Z-VAD-fmk.

(H) Cell cycle distributions of A549 cells treated with 10 M cisplatin (CDDP) alone or in combination with pyridoxine (PN) and/or N-acetylcysteine (NAC). Mean±SEM (n=3). *=p<0.05 (Student's t test), as compared to untreated cells. #=p<0.05 (Student's t test), as compared to cells treated with the same concentration of CDDP. n.s.=non significant (Student's t test), as compared to cells treated with the same concentration of CDDP and NAC.

FIG. 9. Residual clonogenic survival of a panel of Saccharomyces cerevisiae strains

Residual clonogenic survival (RCS, normalized to parental untreated cells) of a panel of Saccharomyces cerevisiae strains missing the indicated genes upon treatment with cisplatin (CDDP, upper panel), C₂-ceramide (C₂-CER, middle panel) or cadmium dichloride (CdCl₂, lower panel). Mean±SEM (n=3). *=p<0.05 and **=p<0.01 (Student's t test), as compared to the response of parental cells. See also Materials and Methods of example 1.

FIG. 10. Cell death induced by cisplatin (CDDP) in distinct CDDP-resistant cell lines, in 4 non-small cell lung cancer (NSCLC) cell lines, in cervical carcinoma, in osteosarcoma as well as in colon carcinoma.

(A-C) Cytofluorometric assessment of cell death induced by the indicated sub-apoptotic dose of cisplatin (CDDP) alone or combined with PN in 3 distinct CDDP-resistant A549 clones (A549 #1-3, A), in 4 non-small cell lung cancer (NSCLC) cell lines other than A549 (HCC827, H1299, H1650 and H1975 cells, B), in cervical carcinoma HeLa cells, in osteosarcoma U2OS cells as well as in wild type (WT) or TP53^{-/-} colon carcinoma HCT 116 cells (C). Mean±SEM (n=3). *=p<0.05 (Student's t test), as compared to untreated cells. #=p<0.05 (Student's t test), as compared to cells treated with the same concentration of CDDP. n.s.=non significant (Student's t test), as compared to cells treated with the same concentration of CDDP.

FIG. 11. Immunohistochemical detection of ALDH7A1, ALK, BCL2L1, PDXP and WSB2 Immunohistochemical detection of aldehyde dehydrogenase, family 7, member A1 (ALDH7A1), anaplastic lymphoma kinase (ALK), BCL-XL (BCL2L1), pyridoxal phosphatase (PDXP) and WSB2 expression levels in bioptic specimens from lung cancer patients. Bars depict picture scale.

FIG. 12. Analysis of the distribution of the levels of expression of ALDH7A1, ALK, BCL2L1, PDXP and WSB2

Analysis of the distribution of the levels of expression of aldehyde dehydrogenase, family 7, member A1 (ALDH7A1), anaplastic lymphoma kinase (ALK), BCL-X_{L} (BCL2L1), hepatic lipase (LIPC), pyridoxal kinase (PDXK) and pyridoxal phosphatase (PDXP) and WSB2 in tissue samples from lung cancer patients. Histograms (diagonal) depict the distribution of the expression of each marker. Dot plots (left panels) illustrate the correlation between the expression levels of all possible couples of markers. Cutoff values for patient stratification are indicated by red dashed lines. Spearman coefficients of correlation are reported in the panels on the right of the diagonal.

FIG. 13. No prognostic or predictive value of ALDH7A1, ALK, BCL2L1, PDXP and WSB2 status in lung cancer patients

(A, C, E, G, I) Kaplan-Meier curves for disease-free survival and overall upon stratification of patients according to the median of expression of aldehyde dehydrogenase, family 7, member A1 (ALDH7A1, A), anaplastic lymphoma kinase (ALK, C), BCL-XL (BCL2L1, E), pyridoxal phosphatase (PDXP, G) and WSB2 (I). p values are depicted.

(B, D, F, H, J) Estimated survival curves for lung cancer patients stratified according to the median of ALDH7A1 (B), ALK (D), BCL2L1 (F), PDXP(H) or WSB2 (J) expression, based on a Cox proportional hazard model with chemotherapy marker interaction. The p value of the interaction is depicted.

FIG. 14. Mechanisms of chemosensitization by PN in vitro.

Representative immunofluorescence microphotographs of the activating phosphorylation of ATM (P-ATM) (A) and the phosphorylation of histone 2AX (γ-H2AX) (B) in untreated versus CDDP-treated cells. Kinetic data as obtained upon automatic visual quantification of the % of cells exhibiting more than 5 (C) or 10 (D) bright nuclear spots (DNA damage foci). Untreated cells exhibit no CDDP-DNA adducts and baseline (<5% positive cells) activation of the DNA damage response (not shown). Mean±SEM (n=3). *=p<0.05 (Student's t test), as compared to untreated cells. #=p<0.05 (Student's t test), as compared to cells treated with CDDP only (C,D).

FIG. 15. Bioenergetic and dynamic aspects of PN-mediated chemosensitization.

(A) Atkinson's energy charge of A549 cells treated for 24 h with 25 μM CDDP and PN, alone or in combination. Mean±SEM (n=3). *=p<0.05 and n.s.=non significant (Studen's t test), as compared to untreated cells. #=p<0.05 (Studen's t test), as compared to cells treated with CDDP only. §=p<0.05 (Studen's t test), as compared to cells treated with PN only. See also FIG. 16A-C

(B) Intracellular levels of GSH in A549 cells treated for 24 h with 25 μM CDDP and PN, alone or in combination. Mean±SEM (n=3). *=p<0.05 and n.s.=non significant (Studen's t test), as compared to untreated cells. #=p<0.05 (Student's t test), as compared to cells treated with CDDP only. §=p<0.05 (Student's t test), as compared to cells treated with PN only.

(C) Cytofluorometric assessment of intracellular GSH levels in A549 cells treated for 24 h with the indicated concentration of CDDP, alone or in combination with PN. Columns report the % of cells with low intracellular GSH (MCBlow). Mean±SEM (n=3). *=p<0.05 (Studen's t test), as compared to untreated cells. #=p<0.05 (Studen's t test), as compared to cells treated with the same concentration of CDDP only. §=p<0.05 (Studen's t test), as compared to cells treated with the same concentration of CDDP plus PN. Mean±SEM (n=6).

(D) Intracellular concentration of GSH (normalized to protein content) in lysates from A549 cells incubated for 24 h in the absence or in the presence of 5 mM GSH. n.s.=non significant (Student's t test), as compared to untreated cells.

(E,F) Intracellular levels of CDDP (normalized to protein content) in lysates from untransfected A549 cells (D) or A549 cells transfected with a PDXK-targeting siRNA (E), treated for 24 h with 25 μM CDDP alone or in combination with PN and/or GSH. Mean±SEM (n=6). *=p<0.05 (Studen's t test), as compared to untransfected (D) or siUNR transfected (E) cells treated with CDDP only. n.s.=non significant (Student's t test), as compared to cells treated with the same concentration of GSH (D) or to siPDXK-transfected cells treated with CDDP only (E).

FIG. 16. Intracellular levels of ATP, ADP, and AMP in A549 cells.

(A-C) Intracellular levels of ATP (A), ADP (B), and AMP (C) in A549 cells treated for 24 h with 25 μM CDDP and PN, alone or in combination. Mean±SEM (n=3). *=p<0.05 and n.s.=non significant (Student's t test), as compared to untreated cells. #=p<0.05 (Student's t test), as compared to cells treated with CDDP only. §=p<0.05 (Student's t test), as compared to cells treated with PN only.

FIG. 17. Intracellular levels of carnitine, acetyl-carnitine and propionyl- in A549 cells.

(A-C) Intracellular levels of carnitine (predicted MW=161.11 Da, A), acetyl-carnitine (predicted MW=203.12 Da, B) and propionyl-carnitine (predicted MW=217.14, C) in A549 cells treated for 24 h with 25 μM CDDP and PN, alone or in combination. Mean±SEM (n=3). *=p<0.05 and n.s.=non significant (Student's t test), as compared to untreated cells. #=p<0.05 (Student's t test), as compared to cells treated with CDDP only. §=p<0.05 (Student's t test), as compared to cells treated with PN only.

(D,E) Cytofluorometric quantification of A549 cell death induced by 25 or 75 μM CDDP alone or in combination with PN and/or the indicated concentration of L-carnitine (L-CAR, D) or acetyl-L-carnitine (AL-CAR, E) PI+=dead cells; PI⁻ DiOC3(6) low=dying cells. Mean±SEM (n=3). *=p<0.05 (Student's t test), as compared to untreated cells. #=p<0.05 (Student's t test), as compared to cells treated with the same concentration of CDDP. n.s.=non significant (Student's t test), as compared to cells treated with the same concentration of CDDP and PN.

FIG. 18. Functional characterization of chemosensitization mediated by vitamin B6 in vivo and prognostic value of PDXK status in lung cancer patients.

(A) In vivo growth of murine Lewis lung carcinoma (LLC) cells grafted into immunocompetent C57BL/6 mice (right panel) upon treatment with intraperitoneal cisplatin (CDDP) alone or in combination with pyridoxine (PN). Mean±SEM (n=15-30 mice/group). *=p<0.05 (two-way ANOVA), as compared to PBS-treated mice. #=p<0.05 (two-way ANOVA), as compared to cells treated with CDDP only.

(B) In vivo growth of human A549 cells stably transfected with a control shRNA (shSCR) or with shRNAs for the downregulation of pyridoxal kinase (shPDXK) and xenografted into immunodeficient Swiss nude mice. Ratios indicate incidence of tumor formation. Mean±SEM (n=21-23 mice/group). After day 20, the growth of shPDXK-transfected cells was always significantly different from that of control cells (*=p<0.05, two-way ANOVA, asterisks not shown).

(C) Cytofluorometric assessment of cell death induced by 50 μM cisplatin (CDDP) in A549 cell clones stably transfected with a control shRNA (shSCR) or with a shRNA targeting pyridoxal kinase (shPDXK). Mean±SEM (n=3). *=p<0.05 (Student's t test), as compared to untreated shUNR-transfected cells. #=p<0.05 (Student's t test), as compared to shUNR transfected cells treated with the same concentration of CDDP. The insert depicts the efficiency of shRNA-mediated PDXK depletion. GAPDH abundance was monitored to ensure equal loading of lanes.

(D,E) Specificity of PDXK immunohistochemical staining. Immunoblots depict the efficiency of siRNA-mediated PDXK downregulation. GAPDH levels were monitored to control equal lane loading (D). Immunohistochemical detection of PDXK levels in A549 cells that were transfected with either a control (siUNR) or a PDXK-specific siRNA (siPDXK). Bars indicate picture scale (E).

(F) Immunohistochemical detection of PDXK levels in biopsies from lung cancer patients. Bars indicate picture scale.

(G) Kaplan-Meier curves for disease-free survival and overall upon stratification of patients according to the median of expression of PDXK. p values (log-rank test) are depicted.

### DETAILED DESCRIPTION OF THE INVENTION

Inventors herein below identify particular products (modifiers of a cancer treatment response) the detection or measure (of the expression level) of which, can be performed (i) to predictively determine if a subject having a tumor, will respond to a treatment of cancer, in particular to a conventional treatment of cancer, or will be resistant to said treatment, and/or (ii) to assess the prognosis of a cancer in a subject having a tumor, i.e., to assess whether the subject will survive to the cancer or die from the cancer.

Inventors in particular identify below methods which can be used to determine the presence, functional expression, or level of expression of the previously mentioned particular products in a biological sample of a subject having a tumor.

In the context of the present invention, the patient or subject is a mammal having a tumor. In a particular embodiment, the mammal is a human being, whatever its age or sex. Unless otherwise specified in the present disclosure, the tumor is a malignant or cancerous tumor.

In the context of the present invention, a “conventional treatment of cancer” may be selected from a chemotherapy, a radiotherapy, an hormonotherapy, an immunotherapy, a specific kinase inhibitor-based therapy, an antiangiogenic agent based-therapy, an antibody-based therapy, in particular a monoclonal antibody-based therapy, and surgery. The term “conventionally” means that the therapy is applied or, if not routinely applied, is appropriate and at least recommended by health authorities. The “conventional” treatment is selected by the cancerologist depending on the specific cancer to be prevented or treated.

The conventional treatment is typically a chemotherapy. In the context of a conventional chemotherapy, the treatment may use a cytotoxic agent or cell death inducer (chemotherapeutic agent), in particular a genotoxic agent. The chemotherapy may use a drug selected from an anthracyclin, a platin, a taxane and an antimitotic agent. In a preferred embodiment, the treatment of cancer is a chemotherapy using a drug selected from an alkylating agent such as camptothecin, cyclophosphamide, mechlorethamine, uramustine, melphalan, chlorambucil, ifosfamide, carmustine, lomostine, streoptozocin, busulfan, thiotepa, dacarbazine, mitozolomide, temozolomide, procarbazine, altretamine, dacarbazine, or an alkylating-like agent, in particular a platin (or platinum-based agent) selected from cis-platinum (cisplatin or CDDP), carboplatin, nedaplatin, satraplatin and oxali-platinum (oxaliplatin or OXP), most preferably a platinum-based agent, in particular CDDP.

The herein described methods of the invention may be applied before and/or after exposition of the subject to the conventional treatment of cancer. It is preferably applied after exposition of the subject to the conventional treatment of cancer. In a particular embodiment, the method of the invention is applied in a subject in the context of a “cancer adjuvant therapy” or “cancer conventional adjuvant therapy”, this expression being herein understood, as explained previously, as a therapy applied to the subject having a tumor after surgical resection of at least part of said tumor.

In a particular and preferred embodiment of the present invention, the subject is typically a subject undergoing a treatment of cancer, in particular a conventional treatment of cancer (in particular a chemotherapy, for example a “cancer adjuvant chemotherapy”). This means that, typically, before assessing the prognosis of a cancer in a subject, or before assessing the sensitivity of the subject to a particular treatment of cancer, this subject has been exposed to said particular treatment of cancer. The subject may have been exposed to part of a complete conventional treatment protocol, for example to at least one cycle of the all treatment protocol, for example two, three or four cycles of the all treatment protocol.

In another particular embodiment of the present invention, the method of assessing the prognosis of a cancer in a subject or the method of assessing the sensitivity of a subject to a treatment of cancer is applied on a subject who has not been previously exposed to a conventional treatment of cancer.

In the present invention, the cancer may be any kind of cancer or neoplasia. The cancer is typically a cancer that is usually or conventionally treated with a chemotherapy and/or surgery. The cancer is preferably selected from non-small cell lung cancer (NSCLC), small cell lung cancer (SCLC), head and neck cancer, cervical carcinoma, ovarian cancer, osteosarcoma, melanoma and colorectal cancer, in particular non-small cell lung cancer (NSCLC).

The term “biological sample” means any biological sample derived from a patient, preferably a sample which contains nucleic acids or proteins. Examples of such samples include fluids, tissues, cell samples, organs, biopsies, etc. Most preferred samples are cancer or tumor tissue samples, in particular lung, skin, intestine, in particular colorectal, uterus, ovary, bone or head and neck tumor samples. Blood, plasma, serum, saliva, urine, seminal fluid, and bronchoalveolar lavage (BAL), etc, may also be used. Cancer cells from a metastase or cancer cells obtained from blood as circulating tumor cells may also be used.

The biological sample of the subject is preferably selected from a tumor sample, a blood sample, a serum sample and a bronchoalveolar lavage (BAL).

The biological sample may be treated prior to its use, e.g. in order to render nucleic acids or proteins available. Techniques of cell lysis, concentration or dilution of nucleic acids or proteins, are known by the skilled person.

An in vitro or ex vivo method of diagnosing, assessing or monitoring the sensitivity of a subject having a tumor to a treatment of cancer (in other words of predicting or determining the susceptibility of a patient having a tumor to respond to a treatment of cancer), in particular to a chemotherapy, is herein provided as a particular embodiment. This method comprises a step of determining, in a biological sample of said subject, the absence, presence or expression level of the expression product (mRNA or protein in particular) of at least one gene selected in particular from the genes herein below identified in Table I, or a step of detecting an alteration, such as a single nucleotide polymorphism (SNP), in at least one of said genes, thereby assessing or monitoring whether the subject having a tumor is responsive or resistant to the treatment of cancer.

Within the context of this invention, “non-responder” or “resistant” refers to the phenotype of a subject who does not respond to a treatment of cancer, in particular to a conventional treatment of cancer as herein defined, i.e. the volume of the tumor does not substantially decrease, or the symptoms of the cancer in the subject are not alleviated, or the cancer progresses, for example the volume of the tumor increases and/or the tumor generates local or distant metastasis. The terms “non-responder” or “resistant” also refer to the phenotype of a subject who will die from the cancer [the detected or measured parameter, for example the expression product of a gene as herein disclosed has a negative impact on the “overall survival” (OS)].

Within the context of this invention, “responder”, “responsive” or “sensitive” refers to the phenotype of a patient who responds to a treatment of cancer, in particular to a conventional treatment of cancer as previously defined, i.e. the volume of the tumor is decreased, at least one of his symptoms is alleviated, or the development of the cancer is stopped, or slowed down.

Typically, a subject who responds to a cancer treatment is a subject who will be completely treated (cured), i.e., a subject who will survive to the cancer [the detected or measured parameter (for example the expression product of gene as herein disclosed) has a beneficial impact on the “overall survival” (OS)].

A subject who responds to a cancer treatment is also, in the sense of the present invention, a subject who typically has a much longer disease free survival (DFS) chance than a patient who has been identified, with a method as herein described, as resistant to a treatment of cancer.

The sensitivity or susceptibility of a subject to a treatment of cancer indicates whether the subject is “responder” or “non-responder”, in other words whether the subject will or will not, be at least partially treated (tumor growth retardation or regression), preferably be completely treated (cured), by said cancer treatment.

The expression “predicting” herein refers to the likelihood that a subject will respond or not to a conventional treatment of cancer and also the extent of the response. Predictive methods of the invention can be used clinically to make treatment decisions by choosing the most appropriate treatment modalities for any particular subject. Therefore, the present invention also concerns a method for selecting a subject suffering of a cancer for a treatment with a particular conventional treatment of cancer, comprising determining the expression level of at least one gene selected from the genes listed in Table 1, in a biological sample of said subject, and selecting the subject predicted to be responsive to said treatment.

TABLE 1
siRNA screening hits
Transcripts whose depletion with at least three out of four non-overlapping siRNAs yielded
significant and consistent chemosensitization or cytoprotection against cisplatin (CDDP)-induced
cell death in A549 cells. Gene ID, accession number, score and Δ are reported.

| Name | Gene ID | Accession number | Score* | Δ** |
|---|---|---|---|---|
| LIPC | 3990 | NM_000236 (SEQ ID NO: 1) | 3 | −13.79 |
| PDXK | 8566 | NM_003681 (SEQ ID NO: 3) | 3 | +7.06 |
| BCL2L1/BCL-X_{L} | 598 | NM_001191 | 3 | −17.41 |
| WSB2 | 55884 | NM_018639 | 4 | −15.79 |
| RAC2 | 5880 | NM_002872 | 3 | −14.91 |
| LRMP | 4033 | NM_006152 | 3 | −13.67 |
| USP8 | 9101 | NM_005154 | 3 | −13.60 |
| MAT2A | 4144 | NM_005911 | 3 | −13.51 |
| CYP11B2 | 1585 | NM_000498 | 3 | −12.77 |
| BTNL2 | 56244 | NM_019602 | 3 | −12.60 |
| ZDHHC9 | 51114 | NM_001008222 | 4 | −12.43 |
| TFF1 | 7031 | NM_003225 | 3 | −12.13 |
| PLK1S1/C20orf19 | 55857 | NM_018474 | 3 | −12.10 |
| NANOS1 | 340719 | NM_199461 | 3 | −12.02 |
| LOC389727 | 389727 | Pseudogene | 3 | −11.75 |
| C10ORF92 | 54777 | NM_017609 | 3 | −11.73 |
| MCL1 | 4170 | NM_021960 | 3 | −11.71 |
| IL6R | 3570 | NM_000565 | 3 | −11.67 |
| PPM1J/PPP2CZ | 333926 | NM_005167 | 3 | −11.28 |
| NCOA3 | 8202 | NM_006534 | 3 | −11.16 |
| RRAD | 6236 | NM_004165 | 3 | −10.96 |
| PLD3 | 23646 | NM_012268 | 3 | −10.75 |
| ITGB6 | 3694 | NM_000888 | 4 | −10.70 |
| UBE2T | 29089 | NM_014176 | 3 | −10.70 |
| SEMG2 | 6407 | NM_003008 | 3 | −10.59 |
| HGD | 3081 | NM_000187 | 3 | −10.44 |
| HIRIP3 | 8479 | NM_003609 | 4 | −10.35 |
| EBP | 10682 | NM_006579 | 3 | −10.32 |
| DEFB4 | 1673 | NM_004942 | 3 | −10.25 |
| KHSRP | 8570 | NM_003685 | 3 | −10.19 |
| ADAR | 103 | NM_001111 | 3 | −9.46 |
| ACHE | 43 | NM_000665 | 3 | −9.41 |
| PRSS21 | 10942 | NM_006799 | 3 | −9.37 |
| FLOT2 | 2319 | NM_004475 | 4 | −9.25 |
| LOC390533 | 390533 | Discontinued | 3 | −9.25 |
| BMP7 | 655 | NM_001719 | 4 | −9.13 |
| CLIC1 | 1192 | NM_001288 | 4 | −9.00 |
| CD151 | 977 | NM_004357 | 3 | −8.96 |
| OSTCL/LOC202459 | 202459 | NM_145303 | 3 | −8.85 |
| IAPP | 3375 | NM_000415 | 4 | −8.82 |
| UBE2L3 | 7332 | NM_003347 | 3 | −8.70 |
| CD34 | 947 | NM_001773 | 3 | −8.48 |
| RAB3C | 115827 | NM_138453 | 3 | −8.24 |
| NLRP1 | 22861 | NM_014922 | 3 | −8.13 |
| C1ORF91 | 56063 | NM_019118 | 3 | −7.97 |
| TCTE3 | 6991 | NM_174910 | 3 | −7.73 |
| KIA1161 | 57462 | NM_020702 | 3 | −7.64 |
| NCKAP1 | 10787 | NM_013436 | 3 | −7.63 |
| TMED1 | 11018 | NM_006858 | 3 | −7.51 |
| BAIAP3 | 8938 | NM_003933 | 3 | −7.47 |
| BSN | 8927 | NM_003458 | 3 | −7.40 |
| PPM1B | 5495 | NM_002706 | 3 | −6.98 |
| GUCA1A | 2978 | NM_000409 | 3 | −6.71 |
| SEMA3C | 10512 | NM_006379 | 3 | −6.17 |
| EEF2 | 1938 | NM_001961 | 3 | +6.32 |
| RAB40A | 142684 | NM_080879 | 3 | +6.47 |
| ALDH7A1 | 501 | NM_001182 | 3 | +6.80 |
| LOC440611 | 440611 | Discontinued | 3 | +8.10 |
| HDLBP | 3069 | NM_005336 | 3 | +8.15 |
| LOC442126 | 442126 | Discontinued | 3 | +8.57 |
| ASTL | 431705 | NM_001002036 | 3 | +8.64 |
| SLC22A18AS | 5003 | NM_007105 | 3 | +8.69 |
| C21ORF2 | 755 | NM004928 | 3 | +8.74 |
| RPSAP32/ | 402123 | Pseudogene | 3 | +9.13 |
| LOC402123 | | | | |
| FN3KRP | 79672 | NM_024619 | 3 | +9.76 |
| DHRSX | 207063 | NM_145177 | 3 | +9.96 |
| LOC441115 | 441115 | Discontinued | 3 | +10.31 |
| COX5B | 1329 | NM_001862 | 3 | +10.61 |
| COX7C | 1350 | NM_001867 | 4 | +10.85 |
| LOC440056 | 440056 | Discontinued | 3 | +11.88 |
| MPP6 | 51678 | NM_016447 | 3 | +10.89 |
| SLC39A5 | 283375 | NM_173596 | 3 | +10.96 |
| GRLF1 | 2909 | NM_004491 | 3 | +11.62 |
| ALK | 238 | NM_004304 | 3 | +12.03 |
| COMMD9 | 29099 | NM_014186 | 3 | +12.60 |
| APAF1 | 317 | NM001160 | 4 | +12.66 |
| ZNF878/ | 729747 | NM_001080404 | 3 | +13.13 |
| LOC342972 | | | | |
| FBXO42 | 54455 | NM_018994 | 3 | +13.47 |
| LOC388882 | 388882 | XR_042117 | 3 | +13.52 |
| RNF26 | 79102 | NM_032015 | 3 | +14.55 |
| TRIP4 | 9325 | NM_016213 | 3 | +14.89 |
| PSMD7 | 5713 | NM_002811 | 3 | +16.00 |
| OVCH1 | 341350 | NM_183378 | 3 | +16.24 |
| PSMC5 | 5705 | NM_002805 | 3 | +16.29 |
| SOCS6 | 9306 | NM_004232 | 3 | +17.10 |

*= n° of siRNAs (out of 4 tested) that significantly modulated cisplatin (CDDP)-induced cell death
**= upon background subtraction and inter-plate normalization,
Δ = relative survival of siRNA-transfected cells (WST-1 signal from CDDP treated, siRNA-transfected cells/WST-1 signal from untreated, siRNA-transfected cells) − relative survival of cells transfected with a control siRNA (siUNR) (WST-1 signal from CDDP treated, siUNR-transfected cells/WST-1 signal from untreated, siUNR-transfected cells). Negative and positive Δ values signify chemosensitization and cytoprotection, respectively.

Preferably, the genes, in particular the at least one gene, for example two or three genes, the absence, presence or expression level of which are advantageously to be determined in a biological sample of the subject having a tumor can be selected from the group consisting of ACHE, ADAR, ALDH7A1, ALK, APAF1, ASTL, BAIAP3, BCL2L1, BMP7, BSN, BTNL2, C10ORF92, C1ORF91, C21ORF2, CD151, CD34, CLIC1, COMMD9, COX5B, COX7C, CYP11B2, DEFB4, DHRSX, EBP, EEF2, FBXO42, FLOT2, FN3KRP, GRLF1, GUCA1A, HDLBP, HGD, HIRIP3, IAPP, IL6R, ITGB6, KHSRP, KIA1161, LIPC (sequence NM_{—}000236=SEQ ID NO:1 and corresponding 499 amino acids sequence=SEQ ID NO:2), LOC388882, LOC389727, LOC390533, LOC440056, LOC440611, LOC441115, LOC442126, LRMP, MAT2A, MCL1, MPP6, NANOS1, NCKAP1, NCOA3, NLRP1, OSTCL, OVCH1, PDXK (sequence NM_{—}003681=SEQ ID NO:3 and corresponding 312 amino acids sequence=SEQ ID NO:4), PLD3, PLK1S1, PPM1B, PPM1J, PRSS21, PSMC5, PSMD7, RAB3C, RAB40A, RAC2, RNF26, RPSAP32, RRAD, SEMA3C, SEMG2, SLC22A18A5, SLC39A5, SOCS6, TCTE3, TFF1, TMED1, TRIP4, UBE2L3, UBE2T, USP8, WSB2, ZDHHC9, ZNF878, more preferably from the group consisting of ACHE, ADAR, ALDH7A1, ALK, APAF1, ASTL, BAIAP3, BCL2L1, BMP7, BSN, BTNL2, CD151, CD34, CLIC1, COMMD9, COX5B, COX7C, CYP11B2, DEFB4, DHRSX, EBP, EEF2, FBXO42, FLOT2, FN3KRP, GRLF1, GUCA1A, HDLBP, HGD, HIRIP3, IAPP, IL6R, ITGB6, KHSRP, LIPC, LRMP, MAT2A, MCL1, MPP6, NANOS1, NCKAP1, NCOA3, NLRP1, OSTCL, OVCH1, PDXK, PLD3, PLK1S1, PPM1B, PPM1J, PRSS21, PSMC5, PSMD7, RAB3C, RAB40A, RAC2, RNF26, RPSAP32, RRAD, SEMA3C, SEMG2, SLC22A18A5, SLC39A5, SOCS6, TCTE3, TFF1, TMED1, TRIP4, UBE2L3, UBE2T, USP8, WSB2, ZDHHC9 and ZNF878; and even more preferably from the group consisting of ACHE, ADAR, ALDH7A1, ALK, ASTL, BAIAP3, BMP7, BSN, BTNL2, CD151, CD34, CLIC1, COMMD9, COX5B, COX7C, CYP11B2, DEFB4, DHRSX, EBP, EEF2, FBXO42, FLOT2, FN3KRP, GRLF1, GUCA1A, HDLBP, HGD, HIRIP3, IAPP, IL6R, ITGB6, KHSRP, LIPC, LRMP, MAT2A, MPP6, NANOS1, NCKAP1, NCOA3, NLRP1, OSTCL, OVCH1, PDXK, PLD3, PLK1S1, PPM1B, PPM1J, PRSS21, PSMC5, PSMD7, RAB3C, RAB40A, RAC2, RNF26, RPSAP32, RRAD, SEMA3C, SEMG2, SLC22A18A5, SLC39A5, SOCS6, TCTE3, TMED1, TRIP4, UBE2L3, UBE2T, USP8, WSB2, ZDHHC9, and ZNF878.

Optionally, the method comprises determining the absence, presence or expression level of at least 1, 2, 3, 4, 5, 6, 7, 8, 9, 10, 50, 60, 65, 70, 74, 75, 80 or 85 genes from those identified in Table 1.

When several genes are studied in the method, said genes can be selected in such a way that they comprise at least one, preferably several, for example at least 1, 2, 3, 4, 5, 6, 7, 8, 9, 10, 50, 60, 65, 70, 74, 75, 80 or 85 over-expressed genes, and at least one, preferably several, for example at least 1, 2, 3, 4, 5, 6, 7, 8, 9, 10, 50, 60, 65, 70, 74, 75, 80 or 85, under-expressed ones. Alternatively, they can be selected in such a way that they comprise only over-expressed or under-expressed genes.

In particular, the non expression or under-expression of a gene selected from ALDH7A1, ALK, APAF1, ASTL, C21ORF2, COMMD9, COX5B, COX7C, DHRSX, EEF2, FBXO42, FN3KRP, GRLF1, HDLBP, LOC388882, LOC440056, LOC440611, LOC441115, LOC442126, MPP6, OVCH1, PDXK, PSMC5, PSMD7, RAB40A, RNF26, RPSAP32, SLC22A18A5, SLC39A5, SOCS6, TRIP4, and ZNF878 and/or the over-expression of a gene selected from ACHE, ADAR, BAIAP3, BCL2L1, BMP7, BSN, BTNL2, C10ORF92, C1ORF91, CD151, CD34, CLIC1, CYP11B2, DEFB4, EBP, FLOT2, GUCA1A, HGD, HIRIP3, IAPP, IL6R, ITGB6, KHSRP, KIA1161, LIPC, LOC389727, LOC390533, LRMP, MAT2A, MCL1, NANOS1, NCKAP1, NCOA3, NLRP1, OSTCL, PLD3, PLK1S1, PPM1B, PPM1J, PRSS21, RAB3C, RAC2, RRAD, SEMA3C, SEMG2, TCTE3, TFF1, TMED1, UBE2L3, UBE2T, USP8, WSB2, and ZDHHC9 are indicative of a resistance to a cancer treatment, in particular of a resistance to a chemotherapy, more particularly of a resistance to a chemotherapy using a drug selected from an alkylating agent such as camptothecin, cyclophosphamide, mechlorethamine, uramustine, melphalan, chlorambucil, ifosfamide, carmustine, lomostine, streoptozocin, busulfan, thiotepa, dacarbazine, mitozolomide, temozolomide, procarbazine, altretamine, dacarbazine, or an alkylating-like agent, in particular a platin (or platinum-based agent) selected from cis-platinum (cisplatin or CDDP), carboplatin, nedaplatin, satraplatin and oxali-platinum (oxaliplatin or OXP), most preferably a platinum-based agent, in particular CDDP.

In a particular embodiment of the present invention, a preferred example of the at least one gene the absence, presence or expression level of which is advantageously to be determined in a biological sample of the subject having a tumor, is the hepatic lipase (LIPC) gene.

The presence of LIPC in a biological sample, preferably in a tumor sample of the subject, is indicative of a resistance of the subject to a conventional cancer treatment, and, in the specific context of a chemotherapy, of a deleterious effect thereof on the subject undergoing such a cancer treatment.

The absence of LIPC in a biological sample, preferably in a tumor sample of the subject, is the indication that a chemotherapy will be efficient in the subject.

In a particular aspect, the method previously described of assessing or monitoring the sensitivity of a subject having a tumor to a particular treatment of cancer, advantageously further comprises a step of comparing the expression level of the at least one gene in the biological sample of the subject to a reference expression level, for instance the expression level of the gene in a reference cell, typically a cancer cell, preferably a cell sensitive to the particular treatment of cancer.

Alternatively, reference or control expression levels are determined with a sample of patients or subjects sensitive to the particular treatment.

An over-expressed gene thus herein refers to a gene having an increased expression in comparison to the expression level of this gene in a sensitive cell, and an under-expressed gene herein refers to a gene having a decreased expression in comparison to the expression level of this gene in a sensitive cell. However, the man skilled in art understands that other references can be used. For instance, the invention also contemplates a reference level corresponding to the expression level in a cell resistant to the particular treatment of cancer.

In the context of the herein described methods, the presence, absence, normal/functional/correct expression, abnormal/non functional expression, and/or expression level of a gene can typically be determined (detected and/or measured) by the presence, quantity and/or analysis of the functional expression of protein or mRNA encoded by said genes. Generally, the expression level as determined is a relative expression level.

The expression level of the selected gene(s) can be determined by measuring the amounts of RNA, in particular mRNA, DNA, in particular cDNA, or protein using a variety of techniques well-known by the man skilled in art.

In a particular embodiment, the under-expression of a gene can be indirectly assessed through the determination of the methylation status of its promoter. Indeed, a methylated promoter is indicative of an expression repression, and therefore of an under-expression. On the contrary, an unmethylated promoter is indicative of a normal expression. The methylation state of a promoter can be assessed by any method known by the one skilled in the art, for instance by the methods disclosed in the following documents: Frommer et al (Proc Natl Acad Sci USA. 1992; 89:1827-31) and Boyd et al (Anal Biochem. 2004; 326:278-80).

More preferably, the determination comprises contacting the sample with selective reagents such as probes, primers or ligands, and thereby detecting the presence, or measuring the amount, of proteins or nucleic acids of interest originally in the sample. Contacting may be performed in any suitable device, such as a plate, microtiter dish, test tube, well, glass, column, and so forth. In specific embodiments, the contacting is performed on a substrate coated with the reagent, such as a nucleic acid array or chip or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or proteins of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). For instance, the probes and primers can be selected from the Taqman Applied ones cited in the present application.

The nucleic acid primers or probes used herein may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, et 2006).

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The antibody (Ab) the presence of which is to be determined according to the previously described method may be selected from:
aldehyde dehydrogenase family 7, member A1 (ALDH7A1) antibody (goat antiserum #SC-79398, Santa Cruz Biotechnology Inc., Santa Cruz, USA),
apoptotic peptidase activating factor 1 (APAF1) antibody (mouse monoclonal IgG₂ #MAB868, R&D Systems),
BCL-X_{L} (BCL2L1) antibody (rabbit antiserum #2764; Cell Signaling Technology Inc., Danvers, USA),
caspase-3 (CASP3) antibody (rabbit monoclonal IgG #9665; Cell Signaling Technology Inc.),
caspase-9 (CASP9) antibody (rabbit antiserum #9502; Cell Signaling Technology Inc.),
cysteine conjugate-β lyase (CCBL1) antibody (mouse monoclonal IgG₂a #AB76963, Abcam plc., Cambridge, UK),
cytochrome c oxidase subunit Vb (COX5B) antibody (mouse antiserum #AB88440, Abcam plc.),

cytochrome c oxidase subunit VIIc (COX7C) antibody (goat antiserum #SC-55711, Santa Cruz Biotechnology Inc.),
hepatic lipase (LIPC) antibody (rabbit antiserum #SC-21007, Santa Cruz Biotechnology Inc.),
interleukin 6 receptor (IL6R) antibody (goat antiserum #AB-227-A, R&D Systems),
pyridoxal kinase (PDXK) antibody (rabbit antiserum #AB38208, Abcam plc.),
pyridoxal phosphatase (PDXP) antibody (rabbit monoclonal IgG #4686, Cell Signaling Technology Inc.),
phospho-TP53 (Ser15) antibody (rabbit antiserum #9284; Cell Signaling Technology Inc.),
phospho-TP53 (Ser46) antibody (rabbit antiserum #2521; Cell Signaling Technology Inc.),
TP53 antibody (rabbit antiserum #9282; Cell Signaling Technology Inc.), and
ZDHHC9 antibody (rabbit antiserum #AB74504, Abcam plc.).

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

The expression of a particular gene (from the genes listed in Table 1) is correct if the expressed protein is active or functional, i.e., in the context of the present invention, if a gene selected from ALDH7A1, ALK, APAF1, ASTL, C21ORF2, COMMD9, COX5B, COX7C, DHRSX, EEF2, FBXO42, FN3KRP, GRLF1, HDLBP, LOC388882, LOC440056, LOC440611, LOC441115, LOC442126, MPP6, OVCH1, PDXK, PSMC5, PSMD7, RAB40A, RNF26, RPSAP32, SLC22A18A5, SLC39A5, SOCS6, TRIP4, and ZNF878 is able to directly or indirectly induce or enhance the action of the therapeutic treatment of a cancer in a subject or if a gene selected from ACHE, ADAR, BAIAP3, BCL2L1, BMP7, BSN, BTNL2, C10ORF92, C1ORF91, CD151, CD34, CLIC1, CYP11B2, DEFB4, EBP, FLOT2, GUCA1A, HGD, HIRIP3, IAPP, IL6R, ITGB6, KHSRP, KIA1161, LIPC, LOC389727, LOC390533, LRMP, MAT2A, MCL1, NANOS1, NCKAP1, NCOA3, NLRP1, OSTCL, PLD3, PLK1S1, PPM1B, PPM1J, PRSS21, RAB3C, RAC2, RRAD, SEMA3C, SEMG2, TCTE3, TFF1, TMED1, UBE2L3, UBE2T, USP8, WSB2, and ZDHHC9 is able to directly or indirectly inhibit or reduce the action of the therapeutic treatment of a cancer in a subject.

An in vitro or ex vivo method of assessing the prognosis of a cancer in a subject is further herein disclosed. This method comprises a step of determining, in a biological sample of the subject, the presence, absence or expression level of a compound selected from vitamin B6, pyridoxal (PL), pyridoxal-5-phosphate (PLP), pyridoxamine (PM), pyridoxamine-5′-phosphate, pyridoxine (PN), pyridoxine-5′-phosphate, L-2-aminoadipate and L-2-aminoadipate 6-semialdehyde, and/or of the expression product (as previously defined) of at least one gene selected from the genes identified in Table I, or a derivative product thereof, thereby assessing the prognosis of the cancer in the subject.

In a particular embodiment of the present invention, a preferred example of the at least one gene the absence, presence or expression level of which is advantageously to be determined in a biological sample of the subject having a tumor, is selected from the hepatic lipase (LIPC) gene and the pyridoxal kinase (PDXK) gene.

The presence of LIPC in a biological sample of a subject, preferably in a tumor sample of the subject, is indicative of a favourable prognosis of the cancer in the subject. On the contrary, the absence of LIPC in a biological sample of a subject, is indicative of an unfavourable prognosis of the cancer in the subject.

An over expression of PDXK in a biological sample of the subject, preferably in a tumor sample of the subject, when compared, as explained previously, to a reference expression level is indicative of a favourable prognosis of the cancer in the subject.

The non expression of PDXK in a biological sample of the subject, preferably in a tumor sample of the subject, or a reduced expression thereof when compared to a reference expression level is indicative of an unfavourable prognosis of the cancer in the subject

The method previously described of assessing the prognosis of a cancer in a subject thus advantageously further comprises a step of comparing, as explained previously, the expression level of the at least one gene in the biological sample of the subject to a reference expression level.

The presence, in at least one of the herein identified genes (see Table 1), of an alteration leading to its abnormal expression, may determine the inability of the subject to positively respond to a cancer treatment and/or may be used to assess the prognosis of a cancer in a subject. The alteration may be in a gene locus. The alteration may be a single nucleotide polymorphism (SNP).

The method of assessing or monitoring the sensitivity of a subject having a tumor to a treatment of cancer or of assessing the prognosis of a cancer in a subject, may also consist in detecting the presence of an altered nucleic acid in a biological sample from the subject (as defined previously), the presence of said altered nucleic acid, being indicative of the inability for the subject to respond to a treatment of a cancer.

This detection step may be performed before or after the administration to the subject having the tumor of at least part of the treatment of cancer, typically of at least part of a conventional treatment of cancer as previously explained.

A particular method herein described may comprise the following steps of (a) obtaining from the subject a test sample of tumoral DNA, cDNA or RNA, (b) contacting the test sample with at least one nucleic acid probe, wherein said nucleic acid is complementary to and specifically hybridises with a targeted altered nucleic acid sequence (one of the herein identified sequence) preferably comprising at least one point mutation, in particular a single nucleotide polymorphism (SNP), to form a hybridization sample, (c) maintaining the hybridization sample under conditions sufficient for the specific hybridization of the targeted nucleic acid sequence with the nucleic acid probe to occur, and (d) detecting whether there is specific hybridization of the altered targeted nucleic acid sequence with the nucleic acid probe.

Further herein described is a DNA chip comprising a solid support which carries nucleic acids that are specific to at least one, preferably at least 2, 3, 4, 5, 6, 7, 8, 9, 10, 20, 25, 50, 60, 65, 70, 74, 75, 80 or 85 genes, selected from the genes identified in Table I. The DNA chip can further comprise nucleic acid(s) for control gene, for instance a positive and negative control or a nucleic acid for an ubiquitous gene in order to normalize the results.

Also herein described is a kit for assessing or monitoring the sensitivity of a subject having a tumor to a treatment of cancer, in particular a chemotherapy, or the prognosis of a cancer in a subject, wherein the kit comprises (i) detection means selected from the group consisting of a pair of primers, a probe, at least one antibody specific to the expression product of a gene selected, in particular from the genes identified in Table I, and/or specific to a derivative product thereof, and a DNA chip as herein described, and, optionally, (ii) a leaflet providing the wild-type sequence of the gene and/or the control quantitative expression value corresponding to the expression product of the gene in a control population and/or corresponding to a derivative product thereof.

The kit can further comprise control reagents and other necessary reagents.

The present invention also relates to the use of a DNA chip or a kit of the invention for preparing a kit for (i) predictively determining if a subject having a tumor, will respond to a treatment of cancer, in particular to a conventional treatment of cancer, or will be resistant to said treatment, and/or for (ii) assessing the prognosis of a cancer in a subject having a tumor.

If the subject is identified, using a method according to the present invention, as resistant to a particular treatment of cancer (for example because this subject does not express or abnormally expresses the previously mentioned genes), the method advantageously further comprises a step of selecting a “compensatory molecule”, to be used, alone or in combination with a conventional treatment of cancer as herein defined, in particular with the particular treatment of cancer mentioned previously, as the appropriate therapeutic treatment of cancer for the subject.

A method of selecting an appropriate, preferably optimal, therapeutic treatment of cancer for a subject having a tumor, is therefore in addition herein described, as well as compensatory molecules for use in such a treatment of cancer, preferably in combination with a conventional treatment of cancer, in particular a chemotherapeutic treatment of cancer, in a subject identified, using a method as herein described, as resistant to the conventional treatment of cancer.

In a particular embodiment, the method of selecting an appropriate treatment of cancer for a subject having a tumor, comprises a step of determining the presence, absence or expression level of the hepatic lipase (LIPC) in a biological sample of said subject, the absence of LIPC in the biological sample being the indication that a chemotherapy will be efficient in the subject, the presence of LIPC, or an over expression thereof when compared to a reference expression level, in the biological sample being, on the contrary, the indication that a chemotherapy, in particular a chemotherapy using cis-platinum (cisplatin or CDDP), more particularly as sole treatment, will be detrimental to the subject.

In another particular embodiment, the method comprises a step of determining the presence or absence of LIPC in a biological sample of said subject, the presence of LIPC in the biological sample, or an over expression thereof when compared to a reference expression level, being the indication that another molecule selected from in particular: an inhibitor of lipase such as Orlistat®; an antagonist of IL-6R, such as an IL6- or an IL6R-neutralizing antibody, and/or a DNA damaging chemotherapeutic agent selected from camptothecin, cyclophosphamide, mechlorethamine, uramustine, melphalan, chlorambucil, ifosfamide, carmustine, lomostine, streoptozocin, busulfan, thiotepa, dacarbazine, mitozolomide, temozolomide, procarbazine, altretamine, dacarbazine, or an alkylating-like agent, in particular a platinum-based agent selected from cis-platinum (cisplatin or CDDP), carboplatin, nedaplatin, satraplatin and oxali-platinum (oxaliplatin or OXP), most preferably a platinum-based agent, in particular CDDP, should preferably be administered to the patient together with a conventional treatment of cancer such as a chemotherapy, in particular a chemotherapy using cis-platinum.

In a further particular embodiment, the method comprises a step of determining the expression level of a compound selected from PDXK, PDXP and aldehyde dehydrogenase 7 family, member A1 (ALDH7A1) in a biological sample of said subject, the non expression thereof or a reduced expression thereof, when compared to a reference expression level, being the indication that a compound selected from pyridoxine (PN, vitamin B6), pyridoxal (PL), pyridoxal-5-phosphate (PLP), pyridoxamine (PM), pyridoxamine-5′-phosphate, pyridoxine (PN), and pyridoxine-5′-phosphate (PNP), should preferably be administered to the patient together with a conventional treatment of cancer such as a chemotherapy, in particular a chemotherapy using cis-platinum, typically when the cancer is selected from a NSCLC, a cervical cancer, a carcinoma, a osteosarcoma and a wild-type colorectal cancer.

Further herein disclosed is the use of a compensatory molecule as herein described for improving the treatment of a cancer, preferably for treating the cancer, or to prepare a pharmaceutical composition for improving the treatment of a cancer, preferably for treating the cancer in a subject tested and identified, by a method as herein described, as resistant to a treatment of cancer, typically to a conventional treatment of cancer. Preferably, the pharmaceutical composition further comprises, as a combined preparation, a drug used in a conventional treatment of cancer, for simultaneous, separate or sequential use in the treatment of said cancer.

Herein described is, in addition, a method for preventing or treating a cancer comprising the administration to a subject in need thereof, as previously explained, of a compensatory molecule or compound, preferably together with (in combination with) a distinct therapeutic agent, typically an agent or a drug used in a conventional treatment of cancer (as a combined preparation), the compound and distinct therapeutic agent being simultaneously or separately administered in the context of a unique cancer protocol.

In a particular embodiment of the present invention, the previously described method for treating cancer is performed on a subject having a tumor before surgical resection of at least part thereof. In another particular embodiment of the present invention, the previously described method for treating cancer is performed on a subject having a tumor after surgical resection of at least part thereof (adjuvant chemotherapy).

Herein disclosed is also a method for screening or identifying a compound suitable for improving the treatment of a cancer (also herein identified as “compensatory molecule”) in a subject having a tumor, said method comprising determining the ability of a test compound (i) to modify the expression in particular of at least one gene selected from the genes identified in Table I, or compensate an abnormal or altered expression thereof, or (ii) to improve the treatment of a cancer with a conventional treatment as described previously, or to reduce the development of a resistance during such a conventional treatment of a cancer.

In a particular aspect, the method comprises: 1) providing a cell or cell-line with at least one gene over-expressed and/or under-expressed selected from the group of genes of Table 1; 2) contacting said cell or cell-line with a test compound; 3) determining the expression level of said at least one gene; and, 4) selecting the compound which inhibits the expression or decreases the expression level of the at least one over-expressed gene or increases the expression level of the at least one under-expressed gene.

In another particular aspect, the method comprises: 1) providing a cell or cell-line sensitive to a particular alkylating agent or alkylating-like agent, in particular a platin (for example CDDP), as herein identified; 2) contacting said cell or cell-line with a test compound and the particular alkylating agent or alkylating-like agent; 3) determining the expression level of said at least one gene selected from the genes listed in Table 1, and, 4) selecting the compound which inhibits the appearance of an over-expression and/or the appearance of an inhibition or under-expression of the at least one gene selected from the genes listed in Table 1.

In a further particular aspect, the method comprises a step of detecting and/or measuring the level of expression, in a cell or cell-line, of at least one gene selected from the genes identified in Table I in the presence of a test compound, wherein a modified expression in comparison with the expression obtained in a control cell or cell-line (the same cell or cell-line which has not been exposed to or contacted with the test compound), is indicative of the capacity of said test compound to modify the expression of said at least one gene in said cell or cell-line.

Also herein described is a method for screening a compound usable for treating a cancer, as a compensatory molecule or compound according to the present invention, in a subject having an altered nucleic acid, an altered nucleic acid expression, or an abnormal expression or activity of the protein encoded by said nucleic acid, said method comprising determining in vitro, in vivo or ex vivo the ability of a test compound to (i) restore a functional expression of said altered or abnormal protein (ii) modulate (i.e., induce, increase, or decrease) the expression or activity of said protein, or (iii) modulate the expression or activity of a ligand of said protein.

The compounds identified with one of the herein described screening methods may be used, in the context of the present invention, as compensatory molecules.

Other characteristics and advantages of the invention are given in the following experimental section (with reference to FIGS. 1 to 13), which should be regarded as illustrative and not limiting the scope of the present application.

### EXPERIMENTAL PART

### Example 1

### Identification of Chemotherapeutic Agent Response Modifiers Reveals New Prognostic and Predictive Biomarkers in Cancer

Personalized cancer therapy relies on biomarkers that predict the evolution and therapeutic response of individual tumors. Most on-small cell lung cancers (NSCLC) are treated with platinum-based agents such as cisplatin, yielding highly heterogeneous therapeutic responses. Here, inventors report a genome-wide siRNA-based screening that led to the identification of gene transcripts whose abundance affects the response of NSCLC cells to cisplatin. Functional and pharmacological validation experiments coupled to epistatic analyses led to the identification of two metabolic pathways affecting cisplatin responses in vitro and in vivo. One pathway involves pyridoxal kinase (PDXK), which generates the bioactive form of vitamin B6 and is required for optimal cisplatin responses. The other pathway involves hepatic lipase (LIPC), whose depletion or pharmacological inhibition has chemosensitizing effects. The expression levels of PDXK and LIPC had a prognostic impact on patients with NSCLC. Moreover, low LIPC expression levels were predictive for a positive effect of cisplatin-based adjuvant chemotherapy.

### Materials and Methods

Unless otherwise specified, all experiments were performed in triplicate and repeated at least twice. Data are reported as means±SEM.

### Chemicals and Cell Cultures.

Unless otherwise specified, chemicals were purchased from Sigma-Aldrich (St. Louis, USA), culture media and supplements for cell culture from Gibco-Invitrogen (Carlsbad, USA) and plasticware from Corning (Corning, USA). Anti-human interleukin 6 (IL6, goat antiserum #AB-206-A) and IL6 receptor (IL6R, goat antiserum #AB-227-A) neutralizing antibodies were purchased by R&D Systems (Minneapolis, USA). Orlistat (Xenical®) was obtained from Roche (Basel, Switzerland). The content of each capsule (120 mg) was resuspended in 1 mL 33% (v/v) ethanol in PBS for 30 min and carefully vortexed every 10 min. Undissolved material was removed by two sequential rounds of centrifugation at 12,000 G (5 min, room temperature, RT), supernatants were collected and stored at −80° C. under protection from light until use. In low-throughput experiments, all cell lines were treated with 2.5 mM pyridoxine (PN), exception made for A549 cells that received 5 mM PN.

Human wild type (WT) non-small cell lung cancer (NSCLC) A549 cells and six distinct cisplatin (CDDP)-resistant derivatives (kindly provided by Dr. Catherine Brenner Jan, Université Paris 11, Chatenay Malabry, France) were routinely maintained in Glutamax®-containing DMEM/F12 medium supplemented with 10% fetal bovine serum (FBS), 10 mM HEPES buffer, 100 units/mL penicillin G sodium and 100 μg/mL streptomycin sulfate. Human NSCLC HCC827, H1299, H1650 and H1975 cells were cultured in Glutamax®-containing RPMI medium, supplemented as above. Human cervical carcinoma HeLa and osteosarcoma U2OS cells as well as murine Lewis lung carcinoma (LLC) cells were grown in endotoxin-free Glutamax®-containing DMEM, supplemented as above. Human WT and TP53^{-/-} colon carcinoma cells were maintained in Glutamax®-containing McCoy's 5A supplemented with 10% FBS, 10 mM HEPES buffer, 1 mM sodium pyruvate, 100 units/mL penicillin G sodium and 100 μg/mL streptomycin sulfate. Cell lines were routinely maintained at +37° C. under 5% CO₂ in T175 flasks, and seeded into 10 cm Petri dishes or 6-, 12-, 24- or 96-wells plates 12-24 h before the experiments.

### Transfections.

The genome-wide siRNA library Human Whole Genome siRNA Set Version 1.0 (Qiagen, Hilden, Germany) was used in this study. The sequences of the siRNAs selected for low-throughput validation analyses, as well as those of other siRNAs that were employed in this study are reported in Table 2.

To avoid depleting the siRNA library of selected sequences, siRNAs for validation experiments were purchased as custom molecules from Sigma-Aldrich. For low-throughput experiments, (see below for the transfection protocols employed in the siRNA screen), siRNA were transfected in A549 cells with Oligofectamine™ reagent (Invitrogen), following the manufacturer's instructions. Transfection with a non-targeting siRNA (siUNR) was employed as a negative control condition. Forty-eight hours after transfection, siRNA-mediated target downregulation was assessed by immunoblotting (see below).

Genome-Wide siRNA Screens.

High-throughput operations were carried out by means of a Microlab STAR automated workstation (Hamilton, Reno, USA) placed under a class II safety cabinet. In all screening rounds, each siRNA was tested in triplicates that were distributed to three different plates. Prior to cell seeding, transfection complexes were individually prepared in a V-bottomed 96-well deep well plate. In brief, each siRNA (as retrieved from the Human Whole Genome siRNA Set Version 1.0) was dissolved in a total volume of 122.5 μL serum-free phenol-red free DMEM/F12 medium (final concentration=10 nM, conditions for 7 wells). Similarly, an appropriate amount of HiPerFect® transfection reagent (Qiagen) was diluted in serum-free phenol-red free DMEM/F12 medium (0.25 μL per well). These solutions were allowed to stand at RT for 5-10 min and then 122.5 μL of diluted HiPerFect® were added to each siRNA solution, mixed by automated pipetting and left for another 10-15 min at RT to facilitate complex formation. Meanwhile, A549 cells from maintenance cultures were collected and resuspended in phenol-red free DMEM/F12 medium supplemented with 10% fetal bovine serum (FBS), 10 mM HEPES buffer, 100 units/mL penicillin G sodium and 100 μg/mL streptomycin sulfate at a final concentration of 45,000 cells/mL. Thereafter, 455 μL of such suspension were added on top of the transfection complexes for each siRNA, mixed by automated pipetting and seeded in 100 μL aliquots, in 6 distinct flat-bottomed 96-well plates. In each plate set (6 plates), the following control conditions were included: empty wells (for background subtraction), untransfected cells and untransfected cells destined to treatment (for the control of cell death induction), UNR-transfected cells and UNR-transfected cells destined to treatment (negative control for transfection), untransfected cells destined to lysis (positive control for the assessment of plasma membrane breakdown), cells transfected with a BAK1-targeting siRNA and cells transfected with a BAK1-targeting siRNA destined to treatment (positive control of siRNA-mediated cytoprotection), cells transfected with a EG5-targeting siRNA (positive control of transfection efficiency). Forty-eight hours later, 10 μL of 0.5 mM CDDP (in phenol red-free culture medium) were administered to 3 out of 6 plates (final concentration=50 μM). After additional 24 h, the 3 plates that were not subjected to cell death induction were assessed for both plasma membrane breakdown and residual cell proliferation, while the 3 plates that were treated with cell death-inducing stimuli were monitored for residual proliferation only (see next section).

Colorimetric Assessment of Plasma Membrane Breakdown and Residual Proliferation.

Plasma membrane breakdown was assessed by quantifying the release of the intracellular enzyme lactate dehydrogenase (LDH) in culture supernatants by means of the Cytotoxicity Detection Kit (Roche), according to the manufacturer's instructions. A positive control for plasma membrane rupture was obtained by incubating cells with 0.1% (v/v) Triton® X-100 for 10 min before the beginning of the test.

Cell proliferation was quantified by means of a colorimetric assay based on the reduction of the colorless tetrazolium salt 4-[3-(4-iodophenyl)-2-(4-nitrophenyl)-2H-5-tetrazolio]-1,3-benzene disulfonate (WST-1, from Roche) to formazan (which exhibit an absorbance peak around 450 nm), according to the manufacturer's instructions.

### Mathematical and Statistical Analysis of Screening Results.

Mean WST-1 and LDH signals for each experimental condition (triplicate wells in 3 distinct plates) were obtained by upon background subtraction and inter-plate normalization.

In the first round of screening (2 siRNAs×23,078 transcripts=43,156 siRNAs), siRNAs that per se exerted a significant cytotoxic (inducing an LDH release ≧15% than that provoked by Triton® X-100) or antiproliferative (reducing WST-1 conversion to ≦80% of that of siUNR-transfected cells) effect were excluded from further analysis (score=0). The modulatory effect on CDDP-induced cell death of the remaining siRNAs was estimated by the introduction of the assay-independent indicator A, as previously reported {de La Motte Rouge, 2007; Tajeddine, 2008}. In brief, for each siRNA, residual proliferation was calculated as the ratio between the WST-1 signal of treated, siRNA-transfected cells and that of untreated, siRNA-transfected cells. Δ was then calculated as the difference between the residual proliferation or siRNA-transfected cells and that of control, siUNR-transfected cells. Non-cytotoxic, non-antiproliferative siRNAs were assigned a score of −1 if they were associated with a significantly (Student's t test, p<0.05) negative Δ, of 0 if their Δ was non significant, and of +1 if they displayed a significantly positive Δ. mRNAs were finally ranked by summing the score associated to each of the corresponding siRNAs, and 1,000 transcripts were selected based on the absolute value of Δ amongst mRNAs with a score of either −2 or +2. The modulatory effects of these transcripts on CDDP-induced cell death were subjected to validation in a second round of screening.

In the second round of screen (4 siRNAs×1,000 transcripts=4,000 siRNAs), the effects of individual siRNAs were analyzed and scored as described above. Thus, mRNA scores could assume any integer figure ranging from −4 to +4. 85 mRNAs exhibiting a score with an absolute score value of either 3 or 4 (i.e., −4, −3, +3 and +4) were selected as final hits and validated in functional low-throughout assays.

### Epistatic Screen.

Every possible couple of siRNAs selected amongst the most efficient siRNAs corresponding to each of the 85 hits from the second round of screening plus 7 siRNAs targeting BAK1, BAX, BCL2, CASP2, STAT3, TP53 and VDAC1 (see also Table 2) was tested for its ability to modulate the response of A549 cells to CDDP, as described above. The sole variations in the screening protocol concerned the transfection solution (which contained 10 nM siRNAₓ+10 nM siRNA_{y} rather than 10 nM siRNAₓ only) and the assay outcome (WST-1 conversion only)

The following mathematical model was established for the estimation of the effects of siRNAs (alone or coupled) on CDDP-induced cell death, as compared to the negative control provided by siUNR:

Yᵢⱼₖ=μ×siRNAWellᵢⱼ×siRNACDDPⱼₖ×εᵢⱼₖ

where
siRNAWellᵢⱼ=siRNA and well combined effect
siRNACDDPⱼₖ=siRNA and CDDP combined effect
εᵢⱼₜ=model residues
i=1 to 4280: siRNA alone or siRNA couple
j=well number
k=0,1: untreated or treated plate.

For untreated plates, the CDDP effect is null, implying siRNACDDPⱼ₀=1.

The effect of individual siRNA as compared to that of siUNR was then evaluated by:

siRNACDDPⱼ₁=siRNACDDPₖ₁
j=1 to 92: siRNA alone
k=siUNR.

To estimate the effects of siRNA couples as compared to those of the corresponding siRNAs alone, the following model was established:

W_{xy}=Wₓ+W_{y}+ε_{xy}

where
W_{xy}=siRNAₓ and siRNA_{y} combined effect
Wₓ=effect of siRNAₓ alone
W_{y}=effect of siRNA_{y} alone
ε_{xy}=epistatic effect between siRNA_{X} and siRNA_{y}.

In this model, ε_{xy} can assume the following values:
ε_{xy}=0: absence of epistasis
ε_{xy}<0: negative (alleviating) epistatic effect
ε_{xy}>0: positive (aggravating) epistatic effect.

Each siRNA couple was then subjected to a Student's t test for the hypothesis ε_{xy}=0, and the following distribution was obtained:

ε_{xy}=0 in 2,012 instances (out of 4,186 total siRNA interactions, i.e., the number of combinations of 92 siRNAs taken 2 at a time, ⁹²C₂)
ε_{xy}<0: in 310 instances
ε_{xy}>0: in 1,864 instances.

Epistasis could be further classified as:
masking epistasis, when W_{xy}=Wₓ>W_{y}
partially masking epistasis, when W_{xy}>Wₓ>W_{y}
complete suppression, when W_{xy}=W_{y}<Wₓ
partial suppression, when W_{y}<W_{xy}<Wₓ
coequal interaction, when W_{xy}=Wₓ=W_{y}.

Finally, siRNAs were clustered based on the epistatic effect (ε) by means of the Ward method and the Pearson's correlation, leading to the identification of 4 modules within each of which siRNAs fail to manifest any consistent degree of epistatic interaction (see also FIG. 4 and Table 5).

### Validation Screens.

Validation screens were performed with 1 siRNA×85 targets as described above for the genome-wide screening, exception made for the amount of HiPerFect® transfection reagent per well (ranging from 0.5 to 2 μL, depending on cell type) and for the readout (WST-1 conversion only). In A549 cells, cell death induction was performed with 60 μM betulinic acid, 30 μM C₂-ceramide (C₂-CER), 150 μM carboplatin, 200 nM camptothecin, 40 μM cadmium dichloride (CdCl₂), 50 μM CDDP, 20 μM doxorubicin, 80 μM etoposide, 10 μM mitoxantrone, 3 μM mytomycin C, 80 μM oxaliplatin; 1.5 μM staurosporine or 8 μM thapsigargin. For cell lines other that A549, CDDP was used at the following final concentrations: 130 μM for A549 #1-3, 100 μM for A549 #4 and #5; 160 μM for A549 #6, 120 μM for HCC827, 100 μM for H1299, 80 μM for H1650, 60 μM for H1975, 40 μM for WT HCT 116 and 50 μM for TP53^{-/-} HCT 116 cells.

Δ values were calculated as described above and subjected to unsupervised hierarchical clustering based on the Euclidean distance metric and average linkage clustering by the open-source MultiExperiment Viewer v. 4.1 software (part of the TM4 software suite) {Saeed, 2003}.

### Transcriptome Studies.

NSCLC A549 cells were treated with 100 μM CDDP, 75 μM C₂-CER or 100 μM CdCl₂ for 12 h, or with 50 μM CDDP, C₂-CER or CdCl₂ for 24 h. Thereafter, cells were collected and lysed for RNA extraction of following established procedures {de La Motte Rouge, 2007}. mRNA expression changes were quantified on G4112A 44 k Whole Human Genome Oligo Microarrays (Agilent Technologies, Santa Clara, USA), as previously reported {Castedo, 2006}. mRNA microarrays underwent standardized post-hybridization processing, image acquisition and analysis (see below) {Castedo, 2006}.

Data from microarray experiments were analyzed with the Rosetta Resolver® System (Rosetta Biosoftware, Seattle, USA). The threshold for statistical significance of probe signals (Intensity) was set at p=10⁻⁵. In case of replicate probes for the same transcript, average Intensity was calculated for probes exhibiting a p value <10⁻⁵. For each mRNA, fold change (FC) was then defined as Intensityₛₐₘₚₗₑ/Intensity_{control} when Intensityₛₐₘₚₗₑ>Intensity_{control}, or as—(Intensity_{control}/Intensityₛₐₘₚₗₑ) when Intensityₛₐₘₚₗₑ<Intensity_{control}. Ratios were calculated as Intensityₛₐₘₚₗₑ/Intensity_{control}.

### Cytofluorometry.

To measure apoptosis-related parameters, living cells were labeled for 30 min at +37° C. with 40 nM 3,3′dihexyloxalocarbocyanine iodide (DiOC₆(3), from Molecular Probes-Invitrogen, Eugene, USA), which quantifies the mitochondrial transmembrane potential (Δψₘ), plus 1 μg/mL propidium iodide (PI), which only accumulates in cells with ruptured plasma membrane {Galluzzi, 2009; Galluzzi, 2008; Galluzzi, 2007}. To assess cell cycle distribution, cells were collected, fixed by gentle vortexing in ice-cold 80% (v/v) ethanol (Carlo Erba Reagents, Milano, Italy) and stained with 50 μg/mL PI in 0.1% (w/v) D-glucose in PBS supplemented with 1 μg/mL (w/v) RNAse A {Mondragon, 2009; Mouhamad, 2007}. All cytofluorometric determinations were carried out a FACSCalibur or a FACScan cytofluorometer (BD Biosciences, San Jose, USA) equipped with a 70 μm nozzle.

First line statistical analysis of cytofluorometric results was performed by using the CellQuest™ software (BD Biosciences), by gating on the events characterized by normal forward scatter and side scatter parameters. Apoptosis- and cell cycle-related data were further analyzed with Microsoft Excel (Microsoft Co., Redmond, USA) and statistical significance was assessed by means of two-tailed Student's t test (p<0.05).

### Immunoblotting.

For the preparation of total protein extracts A549 cells were washed with cold PBS and lysed in a buffer containing 1% NP40, 20 mM HEPES (pH 7.9), 10 mM KCl, 1 mM EDTA, 10% glycerol, 1 mM orthovanadate, 1 mM PMSF, 1 mM dithiothreitol, 10 μg/mL aprotinin, 10 μg/mL leupeptin, and 10 μg/mL pepstatin, according to established protocols {Zermati, 2007}. Lysates were separated on pre-cast 4-12% polyacrylamide NuPAGE® Novex® Bis-Tris gels (Invitrogen), electrotransferred to polyvinylidene fluoride (PVDF) membranes (Bio-Rad, Hercules, USA) and probed with primary antibodies specific for aldehyde dehydrogenase family 7, member A1 (ALDH7A1, goat antiserum #SC-79398, Santa Cruz Biotechnology Inc., Santa Cruz, USA), apoptotic peptidase activating factor 1 (APAF1, mouse monoclonal IgG₂ #MAB868, R&D Systems), BCL-X_{L} (BCL2L1, rabbit antiserum #2764; Cell Signaling Technology Inc., Danvers, USA), caspase-3 (CASP3, rabbit monoclonal IgG #9665; Cell Signaling Technology Inc.), caspase-9 (CASP9, rabbit antiserum #9502; Cell Signaling Technology Inc.), cysteine conjugate-β lyase (CCBL1, mouse monoclonal IgG₂a #AB76963, Abcam plc., Cambridge, UK), cytochrome c oxidase subunit Vb (COX5B, mouse antiserum #AB88440, Abcam plc.), cytochrome c oxidase subunit VIIc (COX7C, goat antiserum #SC-55711, Santa Cruz Biotechnology Inc.), hepatic lipase (LIPC, rabbit antiserum #SC-21007, Santa Cruz Biotechnology Inc.), interleukin 6 receptor (IL6R, goat antiserum #AB-227-A, R&D Systems), pyridoxal kinase (PDXK, rabbit antiserum #AB38208, Abcam plc.), pyridoxal phosphatase (PDXP, rabbit monoclonal IgG #4686, Cell Signaling Technology Inc.), phospho-TP53(Ser15) (rabbit antiserum #9284; Cell Signaling Technology Inc.), phospho-TP53(Ser46) (rabbit antiserum #2521; Cell Signaling Technology Inc.), TP53 (rabbit antiserum #9282; Cell Signaling Technology Inc.) and ZDHHC9 (rabbit antiserum #AB74504, Abcam plc.). Equal loading of lanes was checked by probing membranes with an antibody that specifically binds to glyceraldehyde 3-phosphate dehydrogenase (GAPDH, mouse monoclonal IgG₁ #MAB374, Millipore-Chemicon International, Temecula, USA) or to β-actin (rabbit monoclonal IgG #4970; Cell Signaling Technology Inc.). Finally membranes were incubated with suitable secondary IgG conjugated to horseradish peroxidase (Southern Biotech, Birmingham, USA), followed by chemiluminescence detection with the SuperSignal West Pico® reagent and CL-XPosure® X-ray films (both from Thermo Scientific-Pierce, Rockford, USA).

### Immunofluorescence Microscopy for the Detection of CDDP-GG DNA Adducts.

Immunofluorescence staining and measurement of specific DNA platination products was performed essentially as previously described {Dzagnidze, 2007; Liedert, 2006}, with minor modifications. Briefly, cells were spotted onto ImmunoSelect adhesion slides (Squarix GmbH, Marl, Germany), fixed overnight in ice-cold methanol, and subjected to proteolytic digestion with 60 μg/mL pepsin (100 μL per spot, 10 min, 37° C., in a moist chamber) and 40 μg/mL proteinase K (100 μL per spot, 10 min, 37° C., in a moist chamber). Upon blockage of unspecific binding sites with 5% (w/v) non-fat powdered milk in PBS, slides were incubated with a rat primary antibody that specifically recognizes CDDP-GG DNA adducts (RC-18) (at 37° C. for 2 h or at 4° C. overnight). Primary antibodies were revealed with anti-rat Cy3®-labeled antibodies (Molecular Probes-Invitrogen). Incubation in 1 μg/mL (w/v) 4′,6-diamidino-2-phenylindole (DAPI, from Molecular Probes-Invitrogen) in PBS for 30 min at RT was employed for nuclear counterstaining. Images were acquired on an Axioplan fluorescence microscope (Carl Zeiss GmbH, Göttingen, Germany) coupled to a C4880 CCD camera (Hamamatsu Photonics, Herrsching, Germany). For the detection of CDDP-GG DNA adducts by immunofluorescence microscopy, fluorescence signals were measured by quantitative digital image analysis with the ACAS 6.0 Cytometry Analysis System (ACAS II, Asbach, Germany). The levels of adducts in each sample were calculated as arbitrary fluorescence units (AFUs), upon normalization of integrated antibody-derived fluorescence from 200 nuclei/sample to the corresponding DNA content. Data are presented of mean AFUs±SEM from n=3 independent samples.

### Real-Time Measurements of Cell Adherence.

5×10³ A549 cells were seeded in standard culture conditions on 96-well plates coated with gold microelectrodes (E-plates™, Roche) and let adapt and recover growth for 40 h. Thereafter, cells were treated with 50 μM CDDP alone or in association with 5 mM PN for additional 48 h. Since plating, the adherence of cells to the plate was monitored every 15 min (as a function of plate impedance) by means of an xCELLigence™ Real-Time Cell Analyzer (RTCA, from Roche).

Cell adherence was measured in 3 parallel wells for each experimental condition.

Results are reported as mean cell index (CI) values, which constitute an indication of electrode impedance, ±SEM.

### Quantification of Intracellular PN.

A549 cells were cultured in absence or the presence of 5 mM PN for 24 or 48 h, harvested, washed once in cold PBS and pelleted at 800 G for 10 min. Supernatants were discarded and pellets were dissolved in 500 μL of deionized H₂O by vortexing for 1 min followed by sonication for 10 min. Thereafter, lysates were centrifuged at 5,796 G for 10 min and 60 μL of the resulting supernatant were mixed with an equal volume of trichloroacetic acid (TCA). Upon additional centrifugation (5,796 G, 10 min), 50 μL of sample were injected onto the chromatographic column and PN was measured by liquid chromatography with tandem mass spectrometry (LC-MS/MS), as previously described {Johansson, 2010; Midttun, 2009}.

### Immunofluorescence Microscopy for the Detection of DNA Damage-Related Signaling.

Immunofluorescence microscopy determinations of DNA damage-related events were performed as previously described (Vitale et al., 2007; Vitale et al., 2008). Briefly, cells were fixed in 4% (w/v) paraformaldehyde in PBS, permeabilized with 0.1% SDS and immunostained with antibodies recognizing phospho-ATM (Ser1981 of SEQ ID NO: 187) (mouse monoclonal IgG1κ #05-740, Upstate, Billerica, USA) and phospho-H2AX (Ser140 of SEQ ID NO: 188) (γ-H2AX, rabbit antiserum #4411, Trevigen, Gaithersburg, USA). Slides were then incubated with the appropriate Alexa Fluor® 488 conjugates (Molecular Probes-Invitrogen) in the presence of 10 μM Hoechst 33342 (Molecular Probes-Invitrogen) for nuclear counterstaining. Fluorescence images were acquired using an IRE2 microscope equipped with a DC300F camera (both from Leica Microsystems GmbH, Wetzlar, Germany). Image analysis was performed with the open source software Image J (National Institute of Health). The percentage of phospho-ATM and γ-H2AX positivity was assessed by scoring cells that exhibited more than 5 and 10 very bright nuclear spots (DNA damage foci), respectively. Data are presented as mean±SEM from n=3 independent experiments.

### Yeast Strains Assays.

The haploid WT Saccharomyces cerevisiae strain BY4741 (MATa; his3Δ1; leu2Δ0; met15Δ0; ura3Δ0), its diploid counterpart BY4743 (MATa/MATα; his3Δ1/his3Δ1; leu2Δ0/leu2Δ0; MET15/met15Δ0; LYS2/lys2Δ0; ura3Δ0/ura3Δ0) as well as the null mutants employed in this study were obtained from were obtained from Euroscarf (Frankfurt, Germany). For large-scale experiments (see also FIG. 9), yeast cells were cultured on a routine basis at 28° C., on yeast extract peptone dextrose (YEPD) medium containing 1% yeast extract (Difco Laboratories, Detroit, USA), 2% bacto peptone (Difco Laboratories) and 4% D-glucose {Madeo, 2002}. Stationary phase cells from an overnight culture (8-9×107 cells/mL) were inoculated in 10 mL of medium at a density of 2.5×105 cells/mL, let grow at 28° C. for additional 4 h (up to a final concentration of approximately 1×106 cells/mL) and treated for further 4 h with 0.5 mM CDDP, 40 μM C2-CER or 0.5 mM CdCl2. Control cultures were treated with an equal volume of solvent: N—N-dimethylformamide (DMF) for CDDP, dimethylsulfoxide (DMSO) for C2-CER and H2O for CdCl2.

To achieve vitamin B6-depleted conditions, WT, Δsno1 and Δsnz1 cells {Rodriguez-Navarro, 2002} were grown in a vitamin B6-free medium composed of 1.7% vitamin-free yeast base (Formedium, Norfolk, UK) supplemented with 1% glucose, all vitamins but PN (according to the composition of the classical yeast nitrogen base, from Formedium), all amino acids, uracil and adenine {Madeo, 2002}. In this case, 0.10 mM CDDP (or an equal amount of DMF) was added shortly before cells ceased to grow (approximately 5 h after inoculation) and maintained for 22 h. When appropriate, WT or Δsnz1 cells were pre-incubated for 1 h with 0.05 μM or 1 mM PLP, 1 mM PN or 1 mM aminooxyacetic acid (AOAA) before CDDP administration.

In all settings, cell number was then quantified by means of a CASY® cell counter (Schärfe System, Reutlingen, Germany) and 500 cells from each experimental condition were seeded on three parallel YEPD plates. Following 3 days of incubation at 28° C., plates were finally subjected to the quantification of colony forming units (CFUs). For each genotype or experimental condition, clonogenic survival was calculated as the ratio between the number of CFUs observed in treated and untreated conditions.

### Isolation of Rat Liver Mitochondria and Assessment of Mitochondrial Swelling.

Mitochondria were isolated from the liver of male Wistar Kyoto rats (Charles River Laboratories Inc., Wilmington, USA) by differential centrifugation, as previously described {Zischka, 2008}. Briefly, immediately after organ collection, livers were homogenized with a glass teflon homogenizer in an isolation buffer containing 10 mM triethanolamine, 10 mM acetic acid, 280 mM sucrose, 0.2 mM EGTA (pH adjusted to 7.4 with KOH). Homogenates were cleared from debris and nuclei by two rounds of centrifugation at 750 G (10 min, 4° C.). Mitochondria then were pelleted by centrifugation at 9,000 G (10 min, 4° C.), washed three times in isolation buffer (once at 9,000 G, then at 15,000 G, 10 min, 4° C.) and resuspended in EGTA-free isolation buffer. The integrity and functionality of isolated mitochondria was routinely checked by standard respiratory measurements.

For large-amplitude swelling determinations, mitochondria were resuspended in swelling buffer (10 mM Tris-MOPS pH 7.4, 200 mM sucrose, 5 mM succinate, 1 mM phosphate, 10 μM EGTA and 2 μM rotenone) and pre-incubated for 5 min at RT with swelling buffer (negative control) or either of the following permeability transition inhibitors: 5 or 10 μM bongkrekic acid (BA), 1 or 5 μM cyclosporine A (CsA); 0.1 or 0.5 mM reduced glutathione (GSH) or 0.5 or 2 mM N-acetylcysteine (NAC). Then, swelling buffer (negative control) or either of the following chemicals were added: 100 μM Ca²⁺ (positive control), 50 μM GD3 ganglioside, 100 or 200 μM CDDP, 50 or 100 μM C2-CER or 50 or 100 μM CdCl2. The final assay volume was 200 μL, containing 0.5 mg/mL mitochondria. Permeability transition-induced large-amplitude swelling was assessed by measuring light scattering of mitochondrial suspensions at 540 nm on a standard microplate absorbance reader (μ-Quant™, Bio-Tek, Bad Friedrichshall, Germany) every 5 min over a total assay time of 30 min (at RT), as previously described {Zischka, 2008}.

The following mathematical model has been generated from the entire absorbance dataset:

ABSᵢⱼₜ=μ+αᵢ+repᵢⱼ+βᵢ×t+γᵢ×√t+εᵢⱼₜ

where
i=experiment number
j=replicate number
t=time
εᵢⱼₜ=model residues,
   leading to the definition of the following swelling classes (to which each inhibitor/inducer combination has been assigned):

Class 0 (no swelling)=γᵢ+6.5×βᵢ>−0.01

Class A (linear decrease in absorbance)=γᵢ+6.5×βᵢ≧+0.01 and −0.05≦γᵢ≦0.03

Class B (logarithmic decrease in absorbance)=γᵢ+6.5×βᵢ≧+0.01 and −0.15≦γᵢ≦0.05

Class A and B have been further subdivided according to the amplitude of absorbance decrease (ξ=(ABSₘₐₓ−ABSₘᵢₙ)/ABSₘₐₓ) into:

Subclass 1=ξ<0.35

Subclass 2=ξ≧0.35.

### Mouse Strains.

C57BL/6 (H-2b) and Swiss Nude mice were obtained from Janvier (Le Genest St. Isle, France) and Charles River, respectively. Animals were maintained in pathogen-free conditions, and all experiments were carried out (upon approval by the local ethical committee) according to the Federation of European Laboratory Animal Science Association (FELASA) guidelines. Mice were used between 6 and 12 weeks of age. During the experiments, animals were subjected to artificial light cycle (12 h lights on-12 h lights off) and allowed food and drink ad libitum.

### Tumor Graft Models.

5×10⁵ LLC and A549 cells were subcutaneously xenografted (in 100 μL sterile PBS) in 25-30 immunocompetent (C57BL/6) and immunodeficient (Swiss Nude) mice, respectively. When the surface of tumors reached 25-40 mm², n=4-6 mice were assigned to either of 4 different treatment groups: group 1=100 μL intraperitoneal PBS; group 2=5 mg/kg intraperitoneal CDDP in 100 μL PBS; group 3=240 mg/Kg intraperitoneal Orlistat® in 40 μL PBS; group 4=5 mg/kg intraperitoneal CDDP in 100 μL PBS+intraperitoneal Orlistat® in 40 μL PBS. Treatments were administered three times a week (day 1, 3 and 5; day 8=day 1). Tumor size was monitored on the day of treatment by means of a standard calliper and tumor volume was extrapolated as previously described {Streit, 1999}. Animals bearing tumors that exceeded 20-25% of total body mass or that exhibited large necrotic lesions were sacrificed. Statistical significance was evaluated by two-way ANOVA.

Sample Preparation for RRLC.

A549 cells were left untreated or treated with 25 μM CDDP alone or in combination with 5 mM PN, for 24 h. Thereafter, both attached (living) and detached (dying/dead) cells were harvested, washed once in cold PBS and pelleted (800 G for 5 min). Pellets were resuspended in 200 μL deionized water, briefly vortexed and sonicated. 800 μl ice-cold methanol was added, and samples were kept in ice for 15 min, vortexed and centrifuged at 13,000 G for 10 min at 4° C. Supernatants were dried in a SpeedVac drier (Thermo Scientific-Pierce) and stored at −80° C. until analysis. Pellets were resuspended in 150 μL 98:2 water/acetonitrile immediately before analytical determinations (see below).

Targeted Metabolomic Studies.

ATP, ADP, AMP and GSH were quantified on a Rapid Resolution Liquid Chromatography (RRLC) 1200SL system coupled to a 6410 Triple Quadripole (QQQ) mass spectrometer (both from Agilent Technologies). For nucleotides, RRLC was performed on 150×2.1 mm, 3.5 μM Eclipse Plus columns (Agilent Technologies) with water containing 4 mM dimethylhexylamine (DMHA) plus 0.01% (v/v) acetic acid in channel A and acetonitrile in channel B, in gradient mode at a flow rate of 0.2 mL/min: t=0 min 2% B; t=9 min 32% B; t=10 min 95% B; T=12 min 95% B, re-equilibration time=6 min. Mass spectrometry was performed in positive electrospray ionization mode at +4 kV on the QQQ system operating in MRM mode. MRM transitions were optimized with direct infusions of ATP, ADP, and AMP standards with direct infusion, and four transitions were recorded for each compounds. Parent ions were adduct ions of DMHA and also proton adducts:

ATP 637.2>508 CE (collision energy) 12 V; fragmentor 200 V

637.2>136 CE 44 V

508>410 CE 12 V; fragmentor 150 V

508>136 CE 40 V

ADP 557.2>428 CE 4 V; fragmentor 180 V

557.2>136 CE 40 V

428>348 CE 16 V; fragmentor 145 V

428>136 CE 28 V

AMP 477.2>348 at 4 V; fragmentor 180 V

477.2>136 at 28 V

348>136 CE 20 V; fragmentor 125 V

348>119 CE 45 V

For GSH, RRLC was performed on a 150×2.1 mm, 3.5 μM Stable Bond AQ column (Agilent Technologies) with water containing 0.2% (v/v) acetic acid in channel A and methanol in channel B, in gradient mode at a flow rate of 0.3 mL/min: t=0 min 2% B; t=8 min 95% B; t=9 min 95% B; re-equilibration time=6 min. Mass spectrometry was performed as above. MRM transitions were optimized with direct infusion of GSH standards.

Two Transitions were Recorded:

GSH 308.1>179 CE 8 V; fragmentor 100V

308.1>161.9 CE 12 V; fragmentor 125 V

Data collection and analysis were performed with the MassHunter software (Agilent Technologies).

### Patients.

Tumors resected from 122 patients with NSCLC from the Institut Mutualiste Montsouris (Paris, France) were fixed in neutral buffered 10% formalin solution and paraffin-embedded. Inclusion criteria were: (1) histological diagnosis of NSCLC and (2) complete tumor resection. An informed written consent was obtained from patients according to the local ethical committee. Bioptic specimens were collected in the context of the CHEMORES initiative (an EU funded research collaboration aimed at improving cancer treatment by obtaining increased knowledge on mechanisms of chemotherapy resistance) between 2002 and 2006. The main patients' characteristics (i.e., age, gender, smoking status, tumor histology, follow up, administration of adjuvant chemotherapy) are summarized in Table 6.

### Immunohistochemistry.

Paraffin blocks were sectioned (4-μm thick) on a standard microtome and applied onto histological glass slides (Thermo Fisher Scientific Inc., Waltham, USA). Tissue sections were then deparaffinized in xylene rehydrated by incubation in serial (95%, 70%, 50%, 30%, v/v in PBS) ethanol baths (2 min/bath). Immunostaining was performed following standard procedures {Loriot, 2010; Olaussen, 2006}. Briefly, epitope retrieval was performed by incubating slides in either Tris-EDTA (pH 7.8 or 9), 10 mM citrate buffer (pH 6.0 or pH 7.3) for 30-40 min. Endogenous peroxidase activity was inhibited by treatment with 3% H2O2 for 10 min. Unspecific binding sites were then blocked for 10 min with Protein Block reagent (San Ramon, UA, USA) and slides were incubated for 1 h at RT with primary antibodies that recognizes: ALDH7A1 (rabbit antiserum #AB51029, Abcam plc.; dilution 1:400), ALK (mouse monoclonal IgG₃ #M7195, Dako, Carpinteria, USA; dilution 1:30), BCL2L1 (mouse monoclonal IgG₂a #AHO0222, Invitrogen; dilution 1:50), LIPC (mouse monoclonal IgG₁ #SC-21741, Santa Cruz Biotechnology Inc., dilution 1:600), PDXK (rabbit antiserum #AP7167A, Abgent, San Diego, USA; dilution 1:50), PDXP (rabbit antiserum #HPA001099, Sigma-Aldrich; dilution 1:200) or WSB2 (rabbit antiserum #12124-2-AP, ProteinTech, Chicago, USA; dilution 1:200). Slides were then washed in PBS and incubated for 30 min at RT with secondary antibodies (Vectastain® Universal Elite ABC Kit, from Vector Laboratories Inc., Burlingame, USA). Revelation was performed by the streptavidin-biotin-peroxidase complex method with 3,3′-diaminobenzidine tetrahydrochloride (DAB) as chromogenic substrate. Slides were counterstained with Mayer's hematoxylin (Dako), mounted with glass coverslips (Thermo Fisher Scientific Inc.) and observed by means of a DM2000 microscope equipped with HC PL Fluotar 20×/0.50 and 40×/0.75 objectives and coupled to a DFC280 CCD camera (all from Leica Microsystems GmbH, Wetzlar, Germany).

For each antibody, staining intensity was quantified on a 0-3 scale, using as a reference the signal observed in fibroblasts or endothelial cells (score 2). The percentage of reactive tumor cells was scored on a 0%-100% scale. These variables were combined by calculating each marker's score (ranging from 0-300) as the product between staining intensity (0-3) and the percentage of stained cells (0-100). The cutoff value for patient stratification was arbitrarily chosen as the score median for all markers but LIPC, for which 0 was used (positive versus negative tumors). Outcome variables were overall survival (OS) and disease-free survival (DFS) starting from the surgery date. The objective was to explore whether or not the seven markers carry prognostic and/or predictive information relative to DFS and/or OS. Estimated distributions of time-to-event data are displayed as Kaplan-Meier plots, and survival curves were compared by means of the log-rank test. The Cox proportional hazard model was employed to estimate the interaction between the presence/absence of adjuvant chemotherapy and patient status relative to each marker. Statistical analyses were performed on the open-source software environment for statistical computing and graphics R {Schwarzer, 2007}.

### Results and Discussion

Genome-Wide siRNA Screening for the Identification of New CDDP Response Modifiers.

A genome-wide panel of small interfering RNAs (two siRNAs specific for 23,078 transcripts) was transfected into human NSCLC A549 cells, which then were either left untreated or treated with 50 μM CDDP for 24 hours (which kills ˜45% of cells). Finally, cells were assayed for the conversion of a tetrazolium salt (WST-1, which provides an estimate for cell density/survival) and lactate dehydrogenase (LDH) release (which provides an estimate for plasma membrane breakdown). Cytotoxic (0.4% of all siRNAs) and anti-proliferative (12.2%) siRNAs, per se leading to increased LDH release or decreased WST-1 conversion, respectively, were excluded from further analysis. One thousand transcripts for which both specific siRNAs yielded a concordant and significant modulatory effect on the CDDP-mediated reduction of WST-1 conversion were selected based on their biological potency in the primary screen and subjected to further analysis. Two additional siRNAs were used in further rounds of confirmation, and 85 transcripts for which at least three out of four non-overlapping siRNAs yielded significant and consistent results were selected (FIG. 1A-C, Table 1). 53 of the transcripts are “cytoprotectors” (because their depletion enhances CDDP-induced cell death) and 32 are “chemosensitizers” (because their knockdown reduces CDDP-mediated killing). To confirm the results obtained with WST-1, siRNA-transfected A549 cells were treated with a sub-apoptotic (25 μM) or highly efficient (75 μM) dose of CDDP for 48 h, and then co-stained with the mitochondrial transmembrane potential (Δψₘ)-sensitive dye DiOC₆(3) and the vital dye propidium iodide (PI) to determine the frequency of dying (PI⁻ DiOC₆(3)^{low}) and dead (PI⁺ DiOC₆(3)^{low}) cells (FIG. 1D, FIG. 8A, Table 2). In addition, pharmacological inhibitors specific for druggable siRNA targets were used to validate the screening results (FIG. 1E). For example, both the depletion and inhibition of ZDHHC9 (a palmitoyl transferase of H-RAS and N-RAS), interleukin 6 receptor (IL6R), BCL2L1 (better known as BCL-X_{L}), and hepatic lipase (LIPC) sensitized to CDDP-induced cell death, while the depletion/inhibition of two distinct cytochrome c oxidase (COX) subunits and that of apoptotic peptidase activating factor 1 (APAF1) protected against CDDP (FIG. 1D, E, FIG. 8B). Thus, the screening effort identified known apoptosis modulators (such as BCL-X_{L} and APAF1) as well as proteins that had not previously been implicated in CDDP-induced signalling pathways.

TABLE 2
Main siRNAs used in this study
Sense and antisense sequences of the main siRNAs used in this study. Accession number and
source are provided. Please note that some of most (but not all) of these siRNAs were part
of the Human Whole Genome siRNA Set Version 1.0 (Qiagen, Hilden, Germany).

| Name | Accession number | Sense sequence | Source |
|---|---|---|---|
| siACHE_1 | NM_000665 | 5′-CGCCCTCTGGCTGCAAATAAA-3′(SEQ ID NO: 5) | This study* |
| siACHE_2 | | 5′-CCAGAGTGTCTGCTACCAATA-3′(SEQ ID NO: 6) | |
| siADAR_1 | NM_001111 | 5′-TCCCTCGATAACAGTCAGCTA-3′(SEQ ID NO: 7) | |
| siADAR_2 | | 5′-TTCCGTTACCGCAGGGATCTA-3′(SEQ ID NO: 8) | |
| siALDH7A1_1 | NM_001182 | 5′-TCCGATTCTCTATGTCTTTAA-3′(SEQ ID NO: 9) | |
| siALDH7A1_2 | | 5′-AAGGATGATTGGAGGACCTAT-3′(SEQ ID NO: 10) | |
| siALK_1 | NM_004304 | 5′-CACCTACGTATTTAAGATGAA-3′(SEQ ID NO: 11) | |
| siALK_2 | | 5′-CTCGACCATCATGACCGACTA-3′(SEQ ID NO: 12) | |
| siAPAF1_1 | NM_001160 | 5′-CAGTGAAGGTATGGAATATTA-3′(SEQ ID NO: 13) | |
| siAPAF1_2 | | 5′-TAGGCAGAGTATAAAGTATTA-3′(SEQ ID NO: 14) | |
| siASTL_1 | NM_001002036 | 5′-CAGGCTTTGAAATCAACTTCA-3′(SEQ ID NO: 15) | |
| siASTL_2 | | 5′-TCCCATCTTCAGAAGCAGGAA-3′(SEQ ID NO: 16) | |
| siBAIAP3_1 | NM_003933 | 5′-GTCGACCTTGCTGGACATTAA-3′(SEQ ID NO: 17) | |
| siBAIAP3_2 | | 5′-CTCGCCTGACTCCATCCAGAA-3′(SEQ ID NO: 18) | |
| siBAK1_1 | NM_001188 | 5′-GCGAAGUCUUUGCCUUCUCdTdT-3′(SEQ ID NO: 19) | Qiagen** |
| siBAX_1 | NM_004324 | 5′-GGUGCCGGAACUGAUCAGAdTdT-3′(SEQ ID NO: 20) | (Vitale, |
| siBCL2_1 | NM_000657/NM_000633 | 5′-GCUGCACCUGACGCCCUUCdTdT-3′(SEQ ID NO: 21) | (Vicencio, |
| siBCL2L1_1 | NM_001191 | 5′-AAAGTGCAGTTCAGTAATAAA-3′(SEQ ID NO: 22) | This study* |
| siBCL2L1_2 | | 5′-CAAGCTTTCCCAGAAAGGATA-3′(SEQ ID NO: 23) | |
| siBMP7_1 | NM_001719 | 5′-CCCGGAAGTTCCTGTAATAAA-3′(SEQ ID NO: 24) | |
| siBMP7_2 | | 5′-CTCGTGGAACATGACAAGGAA-3′(SEQ ID NO: 25) | |
| siBSN_1 | NM_003458 | 5′-AAAGGACTTGATATAAACAAA-3′(SEQ ID NO: 26) | |
| siBSN_2 | | 5′-CACGCTGTGTCCCATATGCAA-3′(SEQ ID NO: 27) | |
| siBTNL2_1 | NM_019602 | 5′-ATGGAGGGACATGGAAGGAAA-3′(SEQ ID NO: 28) | |
| siBTNL2_2 | | 5′-CAGAGTGGGAGAAGATATACA-3′(SEQ ID NO: 29) | |
| siC10ORF92_1 | NM_017609 | 5′-CAGGGCCTATTGTCACTTCAA-3′(SEQ ID NO: 30) | |
| siC10ORF92_2 | | 5′-CAGGAGAGGCGAGGACCTGAA-3′(SEQ ID NO: 31) | |
| siC1ORF91_1 | NM_019118 | 5′-ACCGTCTGTCTTCACAAAGTA-3′(SEQ ID NO: 32) | |
| siC1ORF91_2 | | 5′-CTGGCGAGTAGTAGCTGTCAA-3′(SEQ ID NO: 33) | |
| siC21ORF2_1 | NM_004928 | 5′-AAGCGCTTTGTTGGAATGGTA-3′(SEQ ID NO: 34) | |
| siC21ORF2_2 | | 5′-CCGCATGAAGCTGACGCGGAA-3′(SEQ ID NO: 35) | |
| siCASP2_1 | NM_032982/NM_032983 | 5′-ACAGCUGUUGUUGAGCGAAdTdT-3′(SEQ ID NO: 36) | (de La Motte |
| siCCBL1 1 | NM_004059/ | 5′-CCAGUACACCAAGACAUUUdTdT-3′(SEQ ID NO: 37) | This study |
| siCCBL1_2 | NM_001122671 | 5′-GGUCCAGAUCACAUCAUGAdTdT-3′(SEQ ID NO: 38) | |
| siCD151_1 | NM_004357 | 5′-CACCCTGTGCCATCACCATAA-3′(SEQ ID NO: 39) | This study* |
| siCD151_2 | | 5′-CTGCCTGTACAGGAGTCTCAA-3′(SEQ ID NO: 40) | |
| siCD34_1 | NM_001773 | 5′-CACGTGGTGGCTGATACCGAA-3′(SEQ ID NO: 41) | |
| siCD34_2 | | 5′-AACGGCCATTCAGCAAGACAA-3′(SEQ ID NO: 42) | |
| siCLIC1_1 | NM_001288 | 5′-AAGGTGTCTCTCAGAGGAAGT-3′(SEQ ID NO: 43) | |
| siCLIC1_2 | | 5′-TTCCTGCTGTATGGCACTGAA-3′(SEQ ID NO: 44) | |
| siCOMMD9_1 | NM_014186 | 5′-AAGCTCCTGAGTGCAACTTAA-3′(SEQ ID NO: 45) | |
| siCOMMD9_2 | | 5′-CCCGCTGTTCTCACTCATCTT-3′(SEQ ID NO: 46) | |
| siCOX5B_1 | NM_001862 | 5′-CACCTGCACTAAATTACTCAA-3′(SEQ ID NO: 47) | |
| siCOX5B_2 | | 5′-AGGCACCAGGGAAGACCCTAA-3′(SEQ ID NO: 48) | |
| siCOX7C_1 | NM_001867 | 5′-CTAGATATGTTTGTCAATAAA-3′(SEQ ID NO: 49) | |
| siCOX7C_2 | | 5′-CAAGTGGTCGTTACTAGCTAA-3′(SEQ ID NO: 50) | |
| siCYP11B2_1 | NM_000498 | 5′-CACTAGATCATGGGATCCTAA-3′(SEQ ID NO: 51) | This study* |
| siCYP11B2_2 | | 5′-AAGGCGGAACTGTCACTAGAA-3′(SEQ ID NO: 52) | |
| siDEFB4_1 | NM_004942 | 5′-GCCCTAGAAGGTATAAACAAA-3′(SEQ ID NO: 53) | |
| siDEFB4_2 | | 5′-TGCCTTAAGAGTGGAGCCATA-3′(SEQ ID NO: 54) | |
| siDHRSX_1 | NM_145177 | 5′-CCGCGTTAATACAACTGAGTA-3′(SEQ ID NO: 55) | |
| siDHRSX_2 | | 5′-TGCCCTGATAAGCAACTTAAA-3′(SEQ ID NO: 56) | |
| siEBP_1 | NM_006579 | 5′-ACTGGACAACTTTGTACCTAA-3′(SEQ ID NO: 57) | |
| siEBP_2 | | 5′-CACAGTGTGCATGGAAACCAT-3′(SEQ ID NO: 58) | |
| siEEF2_1 | NM_001961 | 5′-CCGCGCCATCATGGACAAGAA-3′(SEQ ID NO: 59) | |
| siEEF2_2 | | 5′-TGCCGTTTCTTTCAATATTTA-3′(SEQ ID NO: 60) | |
| siFBX042_1 | NM_018994 | 5′-CTGGGATCAAATCTCCATTAA-3′(SEQ ID NO: 61) | |
| siFBX042_2 | | 5′-CTCCTGTGTGATAGATGATAA-3′(SEQ ID NO: 62) | |
| siFLOT2_1 | NM_004475 | 5′-CTGCCTGTCCCTTCTGGTAAA-3′(SEQ ID NO: 63) | |
| siFLOT2_2 | | 5′-CACCAAGATTGCTGACTCTAA-3′(SEQ ID NO: 64) | |
| siFN3KR13_1 | NM_024619 | 5′-CCGGACTAGCTTAAGACCAAT-3′(SEQ ID NO: 65) | |
| siFN3KR13_2 | | 5′-ACGAGTGTTCGTGAAAGTGAA-3′(SEQ ID NO: 66) | |
| siGRLF1_1 | NM_004491 | 5′-CAGGATGTTCTGGGAGAGGAA-3′(SEQ ID NO: 67) | |
| siGRLF1_2 | | 5′-CACCACCGAAGAGGTGTTTAA-3′(SEQ ID NO: 68) | |
| siGUCA1A_1 | NM_000409 | 5′-CACCGATACAGTGTTCTCCAA-3′(SEQ ID NO: 69) | |
| siGUCA1A_2 | | 5′-GCCGGTCGTTGTGGACTCTAA-3′(SEQ ID NO: 70) | |
| siHDLBP_1 | NM_005336 | 5′-AAGGATCTAATCATTGAGCAA-3′(SEQ ID NO: 71) | |
| siHDLBP_2 | | 5′-ATCCGTGAAATTCGTGACAAA-3′(SEQ ID NO: 72) | |
| siHGD_1 | NM_000187 | 5′-ACCCTACAAGTACAACCTGAA-3′(SEQ ID NO: 73) | |
| siHGD_2 | | 5′-CGGGAATTATACACCCTACAA-3′(SEQ ID NO: 74) | |
| siHIRIP3_1 | NM_003609 | 5′-TAGGAAGAAACCTGTGGTAAA-3′(SEQ ID NO: 75) | |
| siHIRIP3_2 | | 5′-CAGCCAAAGAGGAGAATCCAA-3′(SEQ ID NO: 76) | |
| siIAPP_1 | NM_000415 | 5′-TTAGAGGACAATGTAACTCTA-3′(SEQ ID NO: 77) | |
| siIAPP_2 | | 5′-ATGCAGGGTATTGCGAAACAA-3′(SEQ ID NO: 78) | |
| siIL6R_1 | NM_000565 | 5′-ACCCAGTTAGCTCTCAAGTTA-3′(SEQ ID NO: 79) | |
| siIL6R_2 | | 5′-CTGCGGGACCATGGAGTGGTA-3′(SEQ ID NO: 80) | |
| siITGB6_1 | NM_000888 | 5′-CTGGAATTACTTGTCAGCCCA-3′(SEQ ID NO: 81) | |
| siITGB6_2 | | 5′-AACCCTTGCAGTAGTATTCCA-3′(SEQ ID NO: 82) | |
| siKHSRP_1 | NM_003685 | 5′-CAAGAGGAGATTGAAACTGAA-3′(SEQ ID NO: 83) | |
| siKHSRP_2 | | 5′-CAGGATTCAGGCTGCAAAGTA-3′(SEQ ID NO: 84) | |
| siKIA1161_1 | NM_020702 | 5′-CAGGCCAGACTTCGTGCCTTA-3′(SEQ ID NO: 85) | |
| siKIA1161_2 | | 5′-CGCGGCCGCCATCAAAGTCAA-3′(SEQ ID NO: 86) | |
| siLIPC_1 | NM_000236 | 5′-CAGGAGAAACCCAGCAAAGAA-3′(SEQ ID NO: 87) | |
| siLIPC_2 | | 5′-CTGAAGACGATCAGAGTCAAA-3′(SEQ ID NO: 88) | |
| siLOC388882_1 | XR_042117 | 5′-CACCTTGGTGAGAATCAGGAA-3′(SEQ ID NO: 89) | |
| siLOC388882_2 | | 5′-CTGCTGTACATGACATCTCTA-3′(SEQ ID NO: 90) | |
| siLOC389727_1 | Pseudogene | 5′-CCACGCAGAGATGACAGCCAA-3′(SEQ ID NO: 91) | |
| siLOC389727_2 | | 5′-CTGGCGGTGAATGACTTTGAA-3′(SEQ ID NO: 92) | |
| siLOC390533_1 | Discontinued | 5′-CGGGTGGTATGGCCAAAGCAT-3′(SEQ ID NO: 93) | |
| siLOC390533_2 | | 5′-ATACAACAGCTTCTTAGGTAA-3′(SEQ ID NO: 94) | |
| siLOC440056_1 | Discontinued | 5′-CCCGAGGCTTCGCTCCGCAAA-3′(SEQ ID NO: 95) | |
| siLOC440056_2 | | 5′-CTCCGGCGCGAGCCTCCCAAA-3′(SEQ ID NO: 96) | |
| siLOC440611_1 | Discontinued | 5′-CACCGAAAGAGTGTTTCCAAA-3′(SEQ ID NO: 97) | |
| siLOC4406112 | | 5′-AAAGAGTGTTTCAAACGTGAA-3′(SEQ ID NO: 98) | |
| siLOC441115_1 | Discontinued | 5′-CAGAGCTACCTCAAACAGGAA-3′(SEQ ID NO: 99) | |
| siLOC441115_2 | | 5′-AGGGAGGTCTTTGAAAGTTCA-3′(SEQ ID NO: 100) | |
| siLOC442126_1 | Discontinued | 5′-CAGGGCGGGCGCAGTCCTGGA-3′(SEQ ID NO: 101) | This study* |
| siLOC442126_2 | | 5′-TCGCACGAGCGGGATACTCCA-3′(SEQ ID NO: 102) | |
| siLRMP_1 | NM_006152 | 5′-CTGGGAAAGTATAGCATGAAA-3′(SEQ ID NO: 103) | |
| siLRMP_2 | | 5′-CACAAACTATATGGTGGTCAA-3′(SEQ ID NO: 104) | |
| siMAT2A_1 | NM_005911 | 5′-CAGCAGGATCCTGATGCCAAA-3′(SEQ ID NO: 105) | |
| siMAT2A_2 | | 5′-CAGGGAGTGTTCCCTATCCAA-3′(SEQ ID NO: 106) | |
| siMCL1_1 | NM_021960 | 5′-CTGGTTTGGCATATCTAATAA-3′(SEQ ID NO: 107) | |
| siMCL1_2 | | 5′-CGGGACTGGCTAGTTAAACAA-3′(SEQ ID NO: 108) | |
| siMPP6_1 | NM_016447 | 5′-TGGAAGGTGTACTAATATATA-3′(SEQ ID NO: 109) | |
| siMPP6_2 | | 5′-CTGATGATTCAGTAAGGTTAA-3′(SEQ ID NO: 110) | |
| siNANOS1_1 | NM_199461 | 5′-CAGGCAGTGCTCAAACTAGAA-3′(SEQ ID NO: 111) | |
| siNANOS1_2 | | 5′-CACCAGTTTACCCATAAGCAA-3′(SEQ ID NO: 112) | |
| siNCKAP1_1 | NM_013436 | 5′-ACGCATGAACTATGTCCAGAA-3′(SEQ ID NO: 113) | |
| siNCKAP1_2 | | 5′-CAGGCATATACTAGTGTCTCA-3′(SEQ ID NO: 114) | |
| siNCOA3_1 | NM_006534 | 5′-CAGGAGGAGATTATAATACTT-3′(SEQ ID NO: 115) | |
| siNCOA3_2 | | 5′-CCGACAGGCACTTGAATTGAA-3′(SEQ ID NO: 116) | |
| siNLRP1_1 | NM_014922 | 5′-CAGCTTCTGCTCGCCAATAAA-3′(SEQ ID NO: 117) | |
| siNLRP1_2 | | 5′-CTGGTTTGCCTTCCAGCACTA-3′(SEQ ID NO: 118) | |
| siOSTCL_1 | NM_145303 | 5′-AAGCATTGGCTCTATGACTGA-3′(SEQ ID NO: 119) | |
| siOSTCL_2 | | 5′-AAAGGCTGTCCAATCCTCTAA-3′(SEQ ID NO: 120) | |
| siOVCH1_1 | NM_183378 | 5′-TACGAGGTGCATTTGGTATAA-3′(SEQ ID NO: 121) | |
| siOVCH1_2 | | 5′-AGCGTGTAATACTGTGCTCAA-3′(SEQ ID NO: 122) | |
| siPDXK_1 | NM_003681 | 5′-TCGAGTGACTTTCTAACCCAA-3′(SEQ ID NO: 123) | |
| siPDXK_2 | | 5′-CCCTGTATTAAGCAAGAATTA-3′(SEQ ID NO: 124) | |
| siPDXP_1 | NM_020315 | 5′-GGUUUGAGGGCCCUUGCAAdTdT-3′(SEQ ID NO: 125) | This study |
| siPDXP_2 | | 5′-GUGAUAGUGACUCAUCAAUdTdT-3′(SEQ ID NO: 126) | |
| siPLD3_1 | NM_012268 | 5′-TTGAGGGAAGATGAAGCCTAA-3′(SEQ ID NO: 127) | This study* |
| siPLD3_2 | | 5′-CCGCATGGTGGACATGCAGAA-3′(SEQ ID NO: 128) | |
| siPLK1S1_1 | NM_018474 | 5′-AAGGAAATATGTGAATCTGAA-3′(SEQ ID NO: 129) | |
| siPLK1S1_2 | | 5′-CAGCGGTGCATTCGAGACAAA-3′(SEQ ID NO: 130) | |
| siPPM1B_1 | NM_002706 | 5′-CAACCAAGTGTTTAGAATGAA-3′(SEQ ID NO: 131) | |
| siPPM1B_2 | | 5′-TAGCCTAACTACACACATCAA-3′(SEQ ID NO: 132) | |
| siPPM1J_1 | NM_005167 | 5′-AACGAGATGGATAAAGGTGAA-3′(SEQ ID NO: 133) | |
| siPPM1L_2 | | 5′-AGGGATGTCCGCTATATCCAA-3′(SEQ ID NO: 134) | |
| siPRSS21_1 | NM_006799 | 5′-CCACTTTGAGTGGATCCAGAA-3′(SEQ ID NO: 135) | |
| siPRSS21_2 | | 5′-ACCCTTGGCCTGTAACAAGAA-3′(SEQ ID NO: 136) | |
| siPSMC5_1 | NM_002805 | 5′-TTGGTCAAGGTACATCCTGAA-3′(SEQ ID NO: 137) | |
| siPSMC5_2 | | 5′-CCGGGTGGCTCTAAGGAATGA-3′(SEQ ID NO: 138) | |
| siPSMD7_1 | NM_002811 | 5′-TTCCGTATTGGTCATCATTGA-3′(SEQ ID NO: 139) | |
| siPSMD7_2 | | 5′-CAGGCCCTAAACTACACAAGA-3′(SEQ ID NO: 140) | |
| siRAB3C_1 | NM_138453 | 5′-GTGGCAAATATGTGATCTTAA-3′(SEQ ID NO: 141) | |
| siRAB3C_2 | | 5′-AAGGACATTTGCCAACAATGA-3′(SEQ ID NO: 142) | |
| siRAB40A_1 | NM_080879 | 5′-ATGGGAGGAAGAAATAGTACA-3′(SEQ ID NO: 143) | |
| siRAB40A_2 | | 5′-TAGGCTGAATAATCTCCTGTA-3′(SEQ ID NO: 144) | |
| siRAC2_1 | NM_002872 | 5′-AAGGAGATTGACTCGGTGAAA-3′(SEQ ID NO: 145) | |
| siRAC2_2 | | 5′-CAATGTGATGGTGGACAGCAA-3′(SEQ ID NO: 146) | |
| siRNF26_1 | NM_032015 | 5′-CACTAGGGTCCAAATACAGAA-3′(SEQ ID NO: 147) | |
| siRNF26_2 | | 5′-GACCTTGGTGTTGGACCTCAA-3′(SEQ ID NO: 148) | |
| siRPSAP32_1 | Pseudogene | 5′-CACTACTGAAATCCTCACTAA-3′(SEQ ID NO: 149) | |
| siRPSAP32_2 | | 5′-CTGACTATCATTACTCTATGA-3′(SEQ ID NO: 150) | |
| siRRAD_1 | NM_004165 | 5′-CGCGGTGGTCTTTGACTGCAA-3′(SEQ ID NO: 151) | This study* |
| siRRAD_2 | | 5′-CCCGGACGCGATGACCCTGAA-3′(SEQ ID NO: 152) | |
| siSEMA3C_1 | NM_006379 | 5′-CAGAATATGATTCACTATTTA-3′(SEQ ID NO: 153) | |
| siSEMA3C_2 | | 5′-TCCCTGAATATTAACAATATA-3′(SEQ ID NO: 154) | |
| siSEMG2_1 | NM_003008 | 5′-CAGGTAACAATTCATAGTCAA-3′(SEQ ID NO: 155) | |
| siSEMG2_2 | | 5′-AAGGCAGTATTTCGATCCAAA-3′(SEQ ID NO: 156) | |
| siSLC22A18AS_1 | NM_007105 | 5′-CTGCAGCAACATAGAGTACAA-3′(SEQ ID NO: 157) | |
| siSLC22A18AS_2 | | 5′-CTCAGCCAGTCTTAAAGGCAA-3′(SEQ ID NO: 158) | |
| siSLC39A5_1 | NM_173596 | 5′-CTCCTGGGACCTCGTCTACTA-3′(SEQ ID NO: 159) | |
| siSLC39A5_2 | | 5′-CAGGCTTCTGTTGCTGGACCA-3′(SEQ ID NO: 160) | |
| siSOCS6_1 | NM_004232 | 5′-TTGATCTAATTGAGCATTCAA-3′(SEQ ID NO: 161) | |
| siSOCS6_2 | | 5′-TAGAATCGTGAATTGACATAA-3′(SEQ ID NO: 162) | |
| siSTAT3_1 | NM_003150 | 5′-CTGGTCTTAACTCTGATTGTA-3′(SEQ ID NO: 163) | |
| siTCTE3_1 | NM_174910 | 5′-TAGAATGGAGCCATTGAAGAA-3′(SEQ ID NO: 164) | |
| siTCTE3_2 | | 5′-CTCAATTGCTGATATAGGTAA-3′(SEQ ID NO: 165) | |
| siTFF1_1 | NM_003225 | 5′-CACCATGGAGAACAAGGTGAT-3′(SEQ ID NO: 166) | |
| siTFF1_2 | | 5′-ACCATCGACGTCCCTCCAGAA-3′(SEQ ID NO: 167) | |
| siTMED1_1 | NM_006858 | 5′-CCGGCCAAAGTCTAGGCAGAA-3′(SEQ ID NO: 168) | |
| siTMED1_2 | | 5′-GAGGAGATGCTGGATGTTAAA-3′(SEQ ID NO: 169) | |
| siTP53_1 | NM_000546 | 5′-GACUCCAGUGGUAAUCUACdTdT-3′(SEQ ID NO: 170) | (Hoffmann, |
| siTRIP4_1 | NM_016213 | 5′-CAGAAATCAGGCGACCATCTA-3′(SEQ ID NO: 171) | This study* |
| siTRIP4_2 | | 5′-CAGCTGCGAATCCAGGATCAA-3′(SEQ ID NO: 172) | |
| siUBE2L3_1 | NM_003347 | 5′-ACCACCGAAGATCACATTTAA-3′(SEQ ID NO: 173) | |
| siUBE2L3_2 | | 5′-AAGGAGCAGCACCAAATCCAA-3′(SEQ ID NO: 174) | |
| siUBE2T_1 | NM_014176 | 5′-TCGCAACTGTGTTGACCTCTA-3′(SEQ ID NO: 175) | |
| siUBE2T_2 | | 5′-AAGAGAGAGCTGCACATGTTA-3′(SEQ ID NO: 176) | |
| siUNR | — | 5′-GCCGGUAUGCCGGUUAAGUdTdT-3′(SEQ ID NO: 177) | (Mondragon, |
| siUSP8_1 | NM_005154 | 5′-CAGGTTCAGGCAAGCCATTTA-3′(SEQ ID NO: 178) | This study* |
| siUSP8_2 | | 5′-AAGGCTCGTATTCATGCAGAA-3′(SEQ ID NO: 179) | |
| siVDAC1_1 | NM_003374/NM_003150 | 5′-GUACGGCCUGACGUUUACAdTdT-3′(SEQ ID NO: 180) | (Tajeddine, |
| siWSB2_1 | NM_018639 | 5′-CACGGCTTCTTACGATACCAA-3′(SEQ ID NO: 181) | This study* |
| siWSB2_2 | | 5′-CACGTTAATTCGGAAGCTAGA-3′(SEQ ID NO: 182) | |
| siZDHHC9_1 | NM_001008222 | 5′-AACCAGATTGTGAAACTGAAA-3′(SEQ ID NO: 183) | |
| siZDHHC9_2 | | 5′-AGCAGGAATGGCAGTAATAAA-3′(SEQ ID NO: 184) | |
| siZNF878_1 | NM_001080404 | 5′-AAGCATTATCTTATCTTGTAA-3′(SEQ ID NO: 185) | |
| siZNF878_2 | | 5′-TAGAGTTGCCTCACAACTTAA-3′(SEQ ID NO: 186) | |

*= Human Whole Genome siRNA Set Version 1.0 (Qiagen, Hilden, Germany)
**= HP Validated siRNA (Qiagen, Hilden, Germany)

### Identification of General Cell Death Modulators Versus Cisplatin (CDDP) Response Modifiers (CRMs).

To discriminate between general modulators of cell death and specific CRMs, inventors characterized the effect of siRNAs identified by our primary screen in A549 cells responding to a phylogenetically conserved stress mediator, ceramide, and an environmental toxin, the heavy metal cadmium. All these agents including CDDP stimulate the intrinsic pathway of apoptosis. However, only cadmium can mediate direct mitochondrion-permeabilizing effects, as determined in cell-free experiments (FIG. 8C-E). CDDP elicited a transcriptional response, as measured in pre-apoptotic conditions, which was clearly different from that induced by ceramide and cadmium (FIG. 2A, B). Among the 53 cytoprotective transcripts (Table 1), only 4 were upregulated and 4 downregulated at 12 h of CDDP treatment (2 and 3 were up- and downregulated, respectively, at 24 h) (FIG. 2C). Moreover, among the 32 chemosensitizing transcripts (Table 1), 2 were upregulated and 5 were downregulated at 12 h (2 and 4 were up- and downregulated, respectively, at 24 h) (FIG. 2C, Table 3).

Next, inventors compared the effects of the 85 siRNAs on the cytotoxic/antiproliferative effects of CDDP, ceramide and cadmium. The biological effects of siRNAs were sorted for CDDP (FIG. 2D) and then plotted in the same order for the other cell death inducers (FIG. 2E, F), clearly demonstrating that most siRNAs modulated inducer-specific rather than general cell death pathways. For example, knockdown of 2 distinct COX subunits protected against, while the depletion of ZDHHC9 and IL6R sensitized to, CDDP only, but not to ceramide and cadmium. On the other hand, knockdown of the essential caspase activator, APAF1, protected against all three cell death inducers. Similarly, the response to CDDP, ceramide and cadmium was assayed in a panel of yeast strains that were deficient for cell death-relevant genes, yielding rather distinct patterns in cell death modulation and hence corroborating the results generated in human cells (FIG. 9).

Next, they tested the 85 siRNAs discovered in A549 cells for their capacity to modulate the CDDP response in six distinct CDDP-resistant A549 clones, four additional NSCLC cell lines and wild-type or TP53^{-/-} HCT 116 colon cancer cells, revealing cell line-specific and cancer-type specific modulations (FIG. 3A, Table 4).

Moreover, on A549 cells responding to 11 different cytotoxic drugs, they found that the 85 siRNAs induced relatively similar modifications in the response to CDDP and carboplatin, yet rather dissimilar changes when CDDP and oxaliplatin were compared (FIG. 4A, Table 4). This method revealed the existence of general (e.g., COX5B, PSMD7, TRIP4) and drug-specific (e.g., ALK, CLIC1, IL6R, etc.) modifiers of the chemotherapeutic response.

TABLE 3
Overlap between siRNA screening hits and the transcriptional signatures of A549
cells treated with cisplatin (CDDP), C2-ceramide (C2-CER) and cadmium dichloride (CdCl2).

| | | | | | CDDP 12 h | | CDDP 24 h | | C2-CER 12 h | | C2-CER 24 h | | CdCl₂ 12 h | | CdCl₂ 24 h | |
|---|---|---|---|---|---|---|---|---|---|---|---|---|---|---|---|---|
| N | Accession | Symbol | Δ* | Score** | p value | Fold change | p value | Fold change | p value | Fold change | p value | Fold change | p value | Fold change | p value | Fold change |
| 1 | NM_018029 | W282 | −15.70 | 4 | n.s. | n.s. | 2.27E−06 | 1.30 | n.s. | n.s. | n.s. | n.s. | 7.93E−07 | 1.32 | n.s. | n.s. |
| 2 | NM_005911 | MAT2A | −13.51 | 3 | 1.58E−30 | −1.97 | n.s. | n.s. | 0.00E+00 | −3.47 | 3.75E−28 | −2.96 | 0.00E+00 | −4.30 | 0.00E+00 | −3.05 |
| 3 | NM_018474 | PLK1S1 | −12.10 | 3 | n.s. | n.s. | n.s. | n.s. | 7.16E−06 | 1.99 | 5.38E−08 | 1.93 | n.s. | n.s. | n.s. | n.s. |
| 4 | NM_372092 | LOC389727 | −11.75 | 3 | n.s. | n.s. | n.s. | n.s. | n.s. | n.s. | n.s. | n.s. | 2.37E−11 | 2.09 | n.s. | n.s. |
| 5 | NM_021960 | MOL1 | −11. [symbol] | 3 | 9.24E−20 | 2.48 | n.s. | n.s. | 1.20E−06 | −1.35 | 2.43E−28 | −1.58 | 1.44E−11 | 1.37 | 1.26E−14 | 1.07 |
| 6 | NM_000565 | IL6R | −11.67 | 3 | n.s. | n.s. | n.s. | n.s. | 4.24E−06 | 2.94 | 1.53E−19 | 3.52 | n.s. | n.s. | n.s. | n.s. |
| 7 | NM_005167 | PPM1J | −11.28 | 3 | 8.27E−06 | 1.63 | n.s. | n.s. | n.s. | n.s. | n.s. | n.s. | n.s. | n.s. | n.s. | n.s. |
| 8 | NM_004185 | RRAD | −10.96 | 3 | 3.45E−31 | 6.59 | 6.32E−18 | 3.75 | n.s. | n.s. | 1.51E−12 | 1.55 | 4.79E−14 | 3.33 | 2.66E−10 | 2.04 |
| 9 | NM_014176 | UBE21 | −10.70 | 3 | n.s. | n.s. | n.s. | n.s. | 0.00E+00 | −2.92 | 1.02E−21 | −4.38 | 1.09E−11 | −2.06 | 2.08E−08 | −1.50 |
| 10 | NM_000187 | HGD | −10.44 | 3 | 2.17E−11 | −1.68 | 6.41E−12 | −1.50 | 0.00E+00 | −2.15 | 0.00E+00 | −4.23 | 3.00E−37 | −2.37 | 1.05E−43 | −2.34 |
| 11 | NM_006579 | EBP | −10.32 | 3 | n.s. | n.s. | n.s. | n.s. | 2.42E−40 | −2.80 | 0.00E+00 | −3.16 | n.s. | n.s. | n.s. | n.s. |
| 12 | NM_003685 | KHSRP | −10.19 | 3 | n.s. | n.s. | 6.37E−14 | −2.00 | 2.21E−10 | −2.29 | 2.51E−08 | −2.23 | n.s. | n.s. | n.s. | n.s. |
| 13 | NM_001111 | ADAR | −9.40 | 3 | n.s. | n.s. | n.s. | n.s. | n.s. | n.s. | 5.61E−08 | −1.45 | n.s. | n.s. | 7.73E−07 | −1.33 |
| 14 | NM_001288 | CLIC1 | −9.00 | 4 | n.s. | n.s. | n.s. | n.s. | n.s. | n.s. | n.s. | n.s. | 7.10E−17 | 4.60 | 4.74E−06 | −1.29 |
| 15 | NM_004357 | CD151 | −8.95 | 3 | n.s. | n.s. | 9.50E−19 | −2.13 | n.s. | n.s. | n.s. | n.s. | n.s. | n.s. | n.s. | n.s. |
| 16 | NM_145303 | OSTCL | −8.85 | 3 | n.s. | n.s. | n.s. | n.s. | 2.27E−10 | 2.25 | 3.29E−27 | 3.08 | n.s. | n.s. | n.s. | n.s. |
| 17 | NM_003347 | UBE2L3 | −8.70 | 3 | 1.70E−30 | −1.76 | n.s. | n.s. | 5.91E−14 | −1.48 | 3.57E−09 | −1.47 | n.s. | n.s. | n.s. | n.s. |
| 18 | NM_006858 | TMED1 | −7.51 | 3 | 1.50E−07 | 1.35 | n.s. | n.s. | n.s. | n.s. | n.s. | n.s. | n.s. | n.s. | n.s. | n.s. |
| 19 | NM_000379 | SEMA3G | −6.17 | 3 | 6.25E−14 | −1.58 | n.s. | n.s. | n.s. | n.s. | n.s. | n.s. | n.s. | n.s. | n.s. | n.s. |
| 20 | NM_001901 | EEF2 | 6.32 | 3 | 2.03E−06 | 1.36 | 1.58E−06 | −1.31 | 2.01E−19 | 2.31 | 0.00E+00 | 2.62 | n.s. | n.s. | n.s. | n.s. |
| 21 | NM_001182 | ALDH7AT | 6.80 | 3 | n.s. | n.s. | 2.37E−27 | 1.62 | 2.25E−25 | −1.85 | 5.74E−36 | −1.98 | 1.43E−19 | −1.65 | 1.27E−30 | −1.82 |
| 22 | NM_003681 | FDXK | 7.00 | 3 | 3.17E−24 | −1.77 | 1.16E−06 | −1.44 | 2.66E−17 | −1.89 | 4.06E−08 | −1.91 | n.s. | n.s. | n.s. | n.s. |
| 23 | NM_005336 | HDLBP | 8.16 | 3 | n.s. | n.s. | n.s. | n.s. | 2.84E−21 | 1.69 | 1.84E−12 | 1.99 | n.s. | n.s. | n.s. | n.s. |
| 24 | NM_007105 | SLC22A16AS | 8.69 | 3 | 1.08E−08 | −1.36 | 7.13E−06 | −1.34 | 1.03E−10 | −1.40 | 1.83E−18 | −1.60 | 1.69E−08 | −1.43 | 6.25E−09 | −1.53 |
| 25 | NM_024619 | FN3KRP | 9.70 | 3 | 5.54E−06 | 1.33 | 4.70E−06 | 1.25 | 9.02E−08 | −1.35 | 1.19E−23 | −1.67 | 1.88E−25 | −3.34 | 1.55E−11 | −2.05 |
| 26 | NM_145177 | DHRSX | 9.90 | 3 | 0.00E−00 | −1.94 | 0.00E+00 | −5.40 | n.s. | n.s. | n.s. | n.s. | 6.54E−15 | 1.64 | 2.62E−06 | 1.38 |
| 27 | NM_001802 | COX5B | 10.61 | 3 | n.s. | n.s. | n.s. | n.s. | 7.50E−26 | −1.62 | 6.29E−15 | −1.61 | n.s. | n.s. | n.s. | n.s. |
| 28 | NM_001867 | COX7C | 10.85 | 4 | n.s. | n.s. | n.s. | n.s. | 8.32E−09 | −1.33 | n.s. | n.s. | n.s. | n.s. | n.s. | n.s. |
| 29 | NM_016447 | MPP6 | 10.89 | 3 | n.s. | n.s. | n.s. | n.s. | n.s. | n.s. | n.s. | n.s. | 7.68E−07 | −3.80 | n.s. | n.s. |
| 30 | NM_014186 | COMMO9 | 12.60 | 3 | n.s. | n.s. | n.s. | n.s. | n.s. | n.s. | n.s. | n.s. | 5.31E−06 | 1.85 | 1.44E−09 | 1.87 |
| 31 | NM_032015 | RNF26 | 14.55 | 3 | n.s. | n.s. | n.s. | n.s. | n.s. | n.s. | 9.51E−04 | −2.11 | 9.46E−08 | −1.77 | 2.45E−09 | −1.51 |
| 32 | NM_016213 | TRIP4 | 14.89 | 3 | 3.33E−06 | −1.64 | n.s. | n.s. | 2.02E−07 | 1.83 | 7.34E−07 | 1.65 | n.s. | n.s. | n.s. | n.s. |
| 33 | NM_002805 | P5MC5 | 16.29 | 3 | n.s. | n.s. | n.s. | n.s. | 3.11E−31 | −2.20 | 5.36E−32 | −2.27 | n.s. | n.s. | n.s. | n.s. |
| 34 | NM_004232 | SOCS6 | 17.10 | 3 | 4.41E−08 | −1.28 | n.s. | n.s. | n.s. | n.s. | n.s. | n.s. | n.s. | n.s. | n.s. | n.s. |

Each siRNA screen hit whose abundance was significantly changed in any of the experimental settings assayed for transcriptome modifications is reported in Table 4 together with accession number, score, Δ, as well as fold change (FC) and p value
*= upon background subtraction and inter-plate normalization, Δ = relative survival of siRNA-transfected cells (WST-1 signal from CDDP treated, siRNA-transfected cells/WST-1 signal from untreated, siRNA-transfected cells) - relative survival of cells transfected with a control siRNA (siUNR) (WST-1 signal from CDDP treated, siUNR-transfected cells/WST-1 signal from untreated, siUNR-transfected cells). Negative and positive Δ values signify chemosensitization and cytoprotection, respectively.
**= n^{o} of siRNAs (out of 4 tested) that significantly modulated cisplatin (CDDP)-induced cell death [symbol]
indicates data missing or illegible when filed

TABLE 4
Effects of the 85 siRNA screen hits in other experimental models of cancer cell death.
Table 4A (in two parts): The 85 siRNAs discovered in A549 cells were assayed for their capacity
to modulate the response to cisplatin (CDDP) in six distinct CDDP-resistant A549 clones (A549
#1-6), four additional NSCLC cell lines (i.e., HCC827, H1299, H1650, H1975) and wild-type or
TP53−/− HCT 116 colon cancer cells.
Table 4A (part I)

| | A549* | | A549 #1 | | A549 #2 | | A549 #3 | | A549 #4 | | A549 #5 | | A549 #6 | |
|---|---|---|---|---|---|---|---|---|---|---|---|---|---|---|
| | Δ** | Δ | Δ | Δ | Δ | Δ | Δ | Δ | Δ | Δ | Δ | Δ | Δ | Δ |
| | (Mean) | (SEM) | (Mean) | (SEM) | (Mean) | (SEM) | (Mean) | (SEM) | (Mean) | (SEM) | (Mean) | (SEM) | (Mean) | (SEM) |
| MCL1 | −0.37 | 0.04 | −0.18 | 0.08 | −0.15 | 0.06 | −0.20 | 0.05 | −0.18 | 0.06 | −0.18 | 0.07 | −0.02 | 0.07 |
| TFF1 | −0.36 | 0.05 | −0.27 | 0.06 | −0.19 | 0.06 | −0.27 | 0.04 | −0.26 | 0.05 | −0.27 | 0.10 | −0.24 | 0.06 |
| BCL2L1 | −0.35 | 0.04 | −0.25 | 0.06 | −0.20 | 0.05 | −0.28 | 0.04 | −0.18 | 0.08 | −0.35 | 0.06 | −0.29 | 0.06 |
| RAB3C | −0.33 | 0.06 | −0.24 | 0.06 | −0.15 | 0.06 | −0.21 | 0.05 | −0.27 | 0.05 | −0.26 | 0.06 | −0.07 | 0.07 |
| PPM1J | −0.33 | 0.04 | −0.22 | 0.06 | −0.19 | 0.06 | −0.22 | 0.05 | −0.25 | 0.06 | −0.20 | 0.08 | −0.07 | 0.06 |
| CD151 | −0.32 | 0.12 | −0.05 | 0.06 | −0.09 | 0.07 | −0.10 | 0.05 | −0.13 | 0.05 | −0.19 | 0.06 | −0.08 | 0.07 |
| BMP7 | −0.30 | 0.04 | −0.13 | 0.05 | −0.10 | 0.06 | −0.17 | 0.05 | −0.11 | 0.09 | −0.11 | 0.10 | 0.02 | 0.06 |
| RAC2 | −0.30 | 0.04 | −0.25 | 0.06 | −0.13 | 0.06 | −0.19 | 0.04 | −0.29 | 0.05 | −0.27 | 0.06 | −0.17 | 0.05 |
| WSB2 | −0.30 | 0.05 | −0.25 | 0.11 | −0.22 | 0.06 | −0.28 | 0.04 | −0.28 | 0.05 | −0.26 | 0.06 | −0.21 | 0.06 |
| LOC390533 | −0.29 | 0.09 | −0.12 | 0.07 | −0.14 | 0.06 | −0.16 | 0.05 | −0.16 | 0.06 | −0.13 | 0.07 | −0.11 | 0.07 |
| CYP11B2 | −0.28 | 0.05 | −0.19 | 0.07 | −0.13 | 0.05 | −0.08 | 0.06 | −0.11 | 0.05 | −0.16 | 0.07 | 0.05 | 0.08 |
| RRAD | −0.28 | 0.05 | −0.16 | 0.09 | −0.11 | 0.07 | −0.17 | 0.06 | −0.22 | 0.05 | −0.27 | 0.07 | −0.06 | 0.07 |
| MAT2A | −0.27 | 0.05 | −0.11 | 0.09 | −0.03 | 0.07 | −0.11 | 0.05 | −0.08 | 0.07 | −0.06 | 0.11 | 0.14 | 0.08 |
| USP8 | −0.27 | 0.04 | −0.24 | 0.05 | −0.20 | 0.06 | −0.01 | 0.07 | 0.20 | 0.11 | 0.03 | 0.14 | −0.01 | 0.08 |
| ITGB6 | −0.27 | 0.05 | −0.09 | 0.08 | −0.08 | 0.07 | −0.10 | 0.08 | −0.06 | 0.07 | −0.21 | 0.07 | 0.02 | 0.06 |
| DEFB4 | −0.27 | 0.04 | −0.19 | 0.06 | −0.10 | 0.06 | −0.02 | 0.09 | 0.07 | 0.05 | −0.03 | 0.13 | 0.06 | 0.08 |
| NCKAP1 | −0.27 | 0.05 | −0.16 | 0.06 | −0.11 | 0.06 | −0.09 | 0.05 | −0.06 | 0.05 | −0.19 | 0.06 | −0.16 | 0.06 |
| IAPP | −0.26 | 0.04 | −0.09 | 0.09 | −0.07 | 0.06 | −0.15 | 0.05 | −0.20 | 0.05 | −0.26 | 0.06 | 0.05 | 0.10 |
| IL6R | −0.25 | 0.04 | −0.11 | 0.06 | −0.06 | 0.06 | −0.09 | 0.06 | 0.08 | 0.08 | −0.05 | 0.10 | 0.08 | 0.07 |
| PLK1S1 | −0.25 | 0.05 | −0.23 | 0.08 | −0.18 | 0.06 | −0.21 | 0.05 | −0.30 | 0.05 | −0.22 | 0.08 | −0.12 | 0.06 |
| BTNL2 | −0.25 | 0.05 | −0.15 | 0.07 | −0.08 | 0.09 | −0.16 | 0.06 | −0.13 | 0.09 | −0.22 | 0.06 | −0.02 | 0.06 |
| FLOT2 | −0.25 | 0.05 | −0.15 | 0.08 | −0.06 | 0.06 | −0.05 | 0.06 | 0.00 | 0.06 | −0.11 | 0.09 | 0.00 | 0.06 |
| SEMG2 | −0.24 | 0.05 | −0.10 | 0.07 | −0.06 | 0.07 | −0.13 | 0.05 | −0.22 | 0.05 | −0.31 | 0.06 | 0.08 | 0.08 |
| BSN | −0.24 | 0.05 | −0.27 | 0.08 | −0.10 | 0.06 | −0.19 | 0.03 | −0.18 | 0.08 | −0.33 | 0.07 | −0.02 | 0.05 |
| PLD3 | −0.24 | 0.05 | −0.15 | 0.06 | −0.12 | 0.06 | −0.11 | 0.05 | 0.01 | 0.10 | −0.12 | 0.08 | −0.11 | 0.07 |
| PRSS21 | −0.24 | 0.04 | −0.23 | 0.06 | −0.13 | 0.06 | −0.11 | 0.07 | −0.07 | 0.07 | −0.01 | 0.22 | −0.09 | 0.09 |
| ADAR | −0.24 | 0.04 | −0.15 | 0.08 | −0.04 | 0.06 | −0.07 | 0.06 | −0.01 | 0.07 | −0.14 | 0.10 | −0.05 | 0.06 |
| HSPC150 | −0.24 | 0.05 | −0.12 | 0.06 | −0.05 | 0.07 | −0.03 | 0.06 | −0.05 | 0.06 | −0.11 | 0.06 | −0.09 | 0.06 |
| HGD | −0.24 | 0.05 | −0.21 | 0.07 | −0.15 | 0.07 | −0.18 | 0.05 | −0.07 | 0.09 | −0.28 | 0.07 | −0.19 | 0.07 |
| CLIC1 | −0.23 | 0.04 | −0.14 | 0.09 | −0.07 | 0.08 | −0.20 | 0.04 | −0.23 | 0.06 | −0.17 | 0.07 | −0.12 | 0.09 |
| NALP1 | −0.22 | 0.04 | −0.14 | 0.10 | −0.09 | 0.05 | −0.13 | 0.05 | −0.12 | 0.07 | −0.04 | 0.08 | 0.07 | 0.06 |
| LRMP | −0.22 | 0.05 | −0.09 | 0.07 | −0.07 | 0.07 | −0.15 | 0.05 | −0.16 | 0.05 | −0.09 | 0.11 | 0.00 | 0.06 |
| C1ORF91 | −0.22 | 0.05 | −0.24 | 0.06 | −0.09 | 0.06 | −0.22 | 0.05 | −0.27 | 0.05 | −0.30 | 0.06 | −0.06 | 0.06 |
| LOC389727 | −0.21 | 0.05 | −0.05 | 0.08 | −0.06 | 0.06 | −0.04 | 0.05 | −0.02 | 0.06 | −0.15 | 0.09 | −0.02 | 0.07 |
| ACHE | −0.21 | 0.05 | −0.18 | 0.08 | −0.05 | 0.08 | −0.12 | 0.07 | −0.17 | 0.06 | −0.08 | 0.11 | −0.03 | 0.07 |
| BAIAP3 | −0.21 | 0.06 | −0.09 | 0.10 | 0.01 | 0.06 | −0.09 | 0.06 | −0.05 | 0.10 | −0.08 | 0.10 | −0.06 | 0.04 |
| HIRIP3 | −0.21 | 0.05 | −0.11 | 0.07 | −0.10 | 0.06 | −0.17 | 0.05 | −0.18 | 0.07 | −0.21 | 0.08 | 0.03 | 0.07 |
| PPM1B | −0.21 | 0.06 | −0.24 | 0.09 | −0.12 | 0.05 | −0.18 | 0.05 | −0.21 | 0.07 | −0.27 | 0.09 | −0.01 | 0.07 |
| KHSRP | −0.21 | 0.05 | −0.19 | 0.07 | −0.16 | 0.06 | −0.18 | 0.06 | −0.19 | 0.08 | −0.27 | 0.07 | 0.00 | 0.06 |
| ZDHHC9 | −0.20 | 0.05 | −0.12 | 0.08 | −0.11 | 0.06 | −0.21 | 0.05 | −0.28 | 0.05 | −0.34 | 0.06 | −0.04 | 0.08 |
| CD34 | −0.20 | 0.05 | −0.17 | 0.07 | −0.07 | 0.06 | −0.14 | 0.05 | −0.20 | 0.05 | −0.24 | 0.07 | −0.02 | 0.06 |
| TMED1 | −0.19 | 0.04 | −0.21 | 0.05 | −0.13 | 0.06 | −0.04 | 0.05 | −0.15 | 0.08 | −0.11 | 0.07 | −0.09 | 0.06 |
| NCOA3 | −0.19 | 0.04 | 0.00 | 0.09 | −0.10 | 0.08 | −0.13 | 0.05 | −0.05 | 0.06 | −0.19 | 0.06 | −0.03 | 0.09 |
| TCTE3 | −0.19 | 0.05 | −0.07 | 0.10 | −0.04 | 0.07 | −0.11 | 0.06 | −0.03 | 0.05 | −0.20 | 0.09 | 0.09 | 0.07 |
| C10orf92 | −0.18 | 0.09 | −0.14 | 0.06 | −0.08 | 0.09 | −0.19 | 0.05 | −0.10 | 0.07 | −0.20 | 0.10 | −0.16 | 0.07 |
| UBE2L3 | −0.17 | 0.04 | −0.21 | 0.06 | −0.10 | 0.06 | −0.12 | 0.05 | −0.02 | 0.07 | −0.10 | 0.07 | −0.07 | 0.07 |
| NANOS1 | −0.17 | 0.06 | −0.03 | 0.07 | −0.02 | 0.06 | −0.02 | 0.06 | −0.14 | 0.05 | −0.04 | 0.07 | −0.06 | 0.08 |
| LIPC | −0.16 | 0.05 | −0.12 | 0.07 | −0.12 | 0.06 | −0.18 | 0.05 | −0.22 | 0.05 | −0.08 | 0.09 | −0.11 | 0.06 |
| OSTCL | −0.16 | 0.07 | 0.03 | 0.07 | 0.03 | 0.07 | 0.04 | 0.06 | 0.05 | 0.07 | −0.06 | 0.07 | 0.01 | 0.06 |
| GUCA1A | −0.14 | 0.07 | −0.11 | 0.09 | 0.01 | 0.10 | −0.02 | 0.05 | 0.05 | 0.07 | −0.02 | 0.08 | 0.01 | 0.05 |
| EBP | −0.13 | 0.06 | −0.13 | 0.06 | −0.11 | 0.06 | −0.08 | 0.05 | −0.07 | 0.07 | −0.04 | 0.12 | −0.12 | 0.06 |
| SEMA3C | −0.11 | 0.06 | −0.18 | 0.08 | −0.07 | 0.06 | −0.03 | 0.04 | −0.17 | 0.07 | −0.28 | 0.08 | 0.01 | 0.05 |
| KIAA1161 | −0.02 | 0.06 | 0.21 | 0.12 | 0.11 | 0.07 | 0.09 | 0.06 | −0.11 | 0.06 | −0.01 | 0.06 | 0.05 | 0.07 |
| ALK | 0.01 | 0.05 | 0.15 | 0.09 | 0.14 | 0.05 | 0.11 | 0.05 | 0.09 | 0.09 | 0.10 | 0.10 | 0.27 | 0.07 |
| EEF2 | 0.02 | 0.06 | 0.13 | 0.10 | 0.26 | 0.20 | 0.34 | 0.11 | 0.32 | 0.09 | 0.24 | 0.09 | 0.14 | 0.06 |
| COX7C | 0.05 | 0.06 | 0.10 | 0.13 | 0.07 | 0.05 | 0.01 | 0.06 | −0.05 | 0.08 | −0.11 | 0.09 | 0.18 | 0.05 |
| C21orf2 | 0.06 | 0.06 | 0.24 | 0.09 | 0.19 | 0.05 | 0.15 | 0.06 | 0.22 | 0.11 | 0.03 | 0.13 | 0.12 | 0.06 |
| RPSAP32 | 0.07 | 0.07 | 0.10 | 0.08 | −0.03 | 0.05 | 0.02 | 0.05 | 0.15 | 0.10 | −0.09 | 0.08 | 0.01 | 0.03 |
| PDXK | 0.07 | 0.07 | 0.16 | 0.12 | 0.17 | 0.09 | 0.21 | 0.05 | 0.22 | 0.08 | 0.05 | 0.08 | 0.17 | 0.05 |
| MPP6 | 0.08 | 0.08 | 0.09 | 0.14 | 0.09 | 0.05 | 0.09 | 0.07 | 0.18 | 0.09 | 0.08 | 0.10 | 0.08 | 0.04 |
| RAB40A | 0.09 | 0.07 | 0.33 | 0.10 | 0.31 | 0.08 | 0.26 | 0.06 | 0.45 | 0.07 | 0.31 | 0.12 | 0.22 | 0.05 |
| OVCH1 | 0.11 | 0.05 | 0.27 | 0.12 | 0.26 | 0.09 | 0.16 | 0.05 | 0.20 | 0.10 | 0.12 | 0.09 | 0.16 | 0.06 |
| GRLF1 | 0.11 | 0.07 | 0.13 | 0.10 | 0.09 | 0.10 | 0.16 | 0.08 | 0.15 | 0.09 | −0.04 | 0.12 | 0.10 | 0.07 |
| SLC22A1LS | 0.11 | 0.08 | 0.32 | 0.13 | 0.19 | 0.07 | 0.30 | 0.08 | 0.56 | 0.17 | 0.35 | 0.13 | 0.23 | 0.03 |
| LOC442126 | 0.12 | 0.07 | 0.24 | 0.18 | 0.29 | 0.16 | 0.30 | 0.07 | 0.44 | 0.13 | 0.39 | 0.12 | 0.23 | 0.06 |
| DHRSX | 0.13 | 0.07 | 0.27 | 0.11 | 0.23 | 0.06 | 0.22 | 0.05 | 0.32 | 0.07 | 0.23 | 0.11 | 0.18 | 0.04 |
| LOC440056 | 0.14 | 0.06 | 0.39 | 0.13 | 0.33 | 0.09 | 0.26 | 0.06 | 0.37 | 0.08 | 0.34 | 0.10 | 0.27 | 0.04 |
| FBXO42 | 0.14 | 0.06 | −0.01 | 0.12 | 0.13 | 0.06 | 0.14 | 0.06 | 0.19 | 0.09 | −0.03 | 0.10 | 0.03 | 0.08 |
| ALDH7A1 | 0.14 | 0.06 | 0.24 | 0.19 | 0.31 | 0.06 | 0.50 | 0.06 | 0.58 | 0.08 | 0.49 | 0.12 | 0.20 | 0.06 |
| LOC440611 | 0.14 | 0.06 | 0.18 | 0.17 | 0.20 | 0.06 | 0.23 | 0.06 | 0.20 | 0.10 | 0.32 | 0.13 | 0.05 | 0.06 |
| HDLBP | 0.15 | 0.06 | 0.27 | 0.17 | 0.24 | 0.08 | 0.27 | 0.12 | 0.42 | 0.08 | 0.34 | 0.12 | 0.17 | 0.05 |
| LOC441115 | 0.16 | 0.07 | 0.19 | 0.17 | 0.32 | 0.08 | 0.15 | 0.05 | 0.15 | 0.09 | 0.08 | 0.09 | 0.07 | 0.07 |
| ASTL | 0.17 | 0.07 | 0.13 | 0.08 | 0.17 | 0.10 | 0.29 | 0.09 | 0.42 | 0.09 | 0.06 | 0.08 | 0.18 | 0.04 |
| FN3KRP | 0.17 | 0.07 | 0.40 | 0.09 | 0.21 | 0.08 | 0.27 | 0.09 | 0.29 | 0.10 | 0.31 | 0.14 | 0.25 | 0.10 |
| RNF26 | 0.18 | 0.07 | 0.24 | 0.11 | 0.16 | 0.07 | 0.28 | 0.07 | 0.49 | 0.13 | 0.40 | 0.11 | 0.13 | 0.07 |
| PSMC5 | 0.18 | 0.09 | 0.22 | 0.12 | 0.19 | 0.10 | 0.38 | 0.05 | 0.53 | 0.13 | 0.39 | 0.10 | 0.33 | 0.08 |
| TRIP4 | 0.20 | 0.07 | 0.32 | 0.13 | 0.30 | 0.13 | 0.36 | 0.07 | 0.41 | 0.08 | 0.42 | 0.08 | 0.29 | 0.06 |
| SOCS6 | 0.21 | 0.08 | 0.31 | 0.23 | 0.27 | 0.08 | 0.14 | 0.09 | 0.25 | 0.15 | −0.02 | 0.08 | 0.17 | 0.06 |
| PSMD7 | 0.22 | 0.05 | 0.23 | 0.16 | 0.39 | 0.07 | 0.25 | 0.08 | 0.49 | 0.12 | 0.22 | 0.08 | 0.33 | 0.17 |
| COX5B | 0.23 | 0.05 | 0.07 | 0.12 | 0.11 | 0.04 | 0.17 | 0.05 | 0.31 | 0.07 | 0.05 | 0.09 | 0.10 | 0.05 |
| SLC39A5 | 0.23 | 0.07 | 0.38 | 0.14 | 0.45 | 0.16 | 0.45 | 0.12 | 0.52 | 0.09 | 0.60 | 0.11 | 0.29 | 0.06 |
| ZNF878 | 0.25 | 0.08 | 0.17 | 0.13 | 0.16 | 0.08 | 0.30 | 0.09 | 0.37 | 0.12 | 0.10 | 0.11 | 0.19 | 0.04 |
| APAF12 | 0.25 | 0.07 | 0.07 | 0.11 | 0.20 | 0.06 | 0.24 | 0.05 | 0.20 | 0.10 | 0.09 | 0.09 | 0.04 | 0.06 |
| LOC388882 | 0.26 | 0.06 | 0.32 | 0.15 | 0.37 | 0.12 | 0.37 | 0.08 | 0.53 | 0.09 | 0.23 | 0.14 | 0.26 | 0.07 |
| COMMD9 | 0.35 | 0.09 | 0.32 | 0.11 | 0.33 | 0.08 | 0.54 | 0.09 | 0.66 | 0.13 | 0.48 | 0.14 | 0.35 | 0.05 |

Table 4A (part II)

| | H1299 | | H1650 | | H1975 | | HCC827 | | HCT 116 | | p53^{-/-} HCT 116 | |
|---|---|---|---|---|---|---|---|---|---|---|---|---|
| | Δ | Δ | Δ | Δ | Δ | Δ | Δ | Δ | Δ | Δ | Δ | Δ |
| | (Mean) | (SEM) | (Mean) | (SEM) | (Mean) | (SEM) | (Mean) | (SEM) | (Mean) | (SEM) | (Mean) | (SEM) |
| MCL1 | −0.13 | 0.08 | 0.00 | 0.12 | −0.06 | 0.07 | −0.05 | 0.13 | −0.10 | 0.06 | −0.12 | 0.10 |
| TFF1 | −0.40 | 0.06 | −0.13 | 0.11 | −0.27 | 0.07 | −0.18 | 0.10 | 0.31 | 0.06 | 0.09 | 0.10 |
| BCL2L1 | −0.23 | 0.06 | −0.14 | 0.09 | −0.08 | 0.07 | −0.31 | 0.11 | 0.04 | 0.05 | −0.05 | 0.10 |
| RAB3C | −0.14 | 0.08 | −0.11 | 0.07 | −0.19 | 0.08 | −0.08 | 0.10 | 0.01 | 0.06 | −0.12 | 0.09 |
| PPM1J | −0.23 | 0.06 | −0.14 | 0.11 | −0.23 | 0.06 | −0.09 | 0.11 | 0.17 | 0.06 | 0.00 | 0.11 |
| CD151 | 0.00 | 0.09 | −0.05 | 0.09 | −0.20 | 0.07 | −0.11 | 0.13 | 0.20 | 0.09 | 0.19 | 0.12 |
| BMP7 | −0.11 | 0.07 | −0.10 | 0.12 | −0.02 | 0.07 | −0.13 | 0.10 | −0.07 | 0.05 | 0.00 | 0.11 |
| RAC2 | −0.25 | 0.10 | −0.14 | 0.06 | −0.17 | 0.08 | −0.18 | 0.10 | 0.11 | 0.04 | 0.03 | 0.10 |
| WSB2 | −0.21 | 0.06 | −0.10 | 0.07 | −0.18 | 0.08 | 0.03 | 0.12 | 0.14 | 0.05 | 0.03 | 0.11 |
| LOC390533 | −0.28 | 0.07 | −0.13 | 0.07 | −0.08 | 0.06 | −0.14 | 0.09 | 0.13 | 0.06 | −0.10 | 0.13 |
| CYP11B2 | −0.19 | 0.06 | −0.12 | 0.08 | −0.10 | 0.09 | 0.07 | 0.11 | 0.05 | 0.05 | −0.02 | 0.13 |
| RRAD | −0.28 | 0.06 | 0.03 | 0.07 | 0.00 | 0.13 | 0.04 | 0.09 | 0.18 | 0.07 | 0.12 | 0.08 |
| MAT2A | −0.06 | 0.06 | 0.00 | 0.13 | −0.11 | 0.10 | 0.21 | 0.12 | 0.30 | 0.05 | 0.31 | 0.10 |
| USP8 | −0.15 | 0.06 | −0.05 | 0.10 | −0.16 | 0.12 | 0.01 | 0.13 | 0.17 | 0.07 | −0.06 | 0.08 |
| ITGB6 | 0.04 | 0.07 | 0.19 | 0.08 | −0.18 | 0.11 | 0.18 | 0.12 | 0.45 | 0.08 | 0.45 | 0.09 |
| DEFB4 | −0.30 | 0.05 | −0.03 | 0.07 | −0.07 | 0.07 | 0.02 | 0.09 | 0.16 | 0.10 | 0.09 | 0.15 |
| NCKAP1 | −0.17 | 0.06 | −0.02 | 0.09 | −0.04 | 0.10 | 0.10 | 0.09 | 0.32 | 0.08 | 0.22 | 0.13 |
| IAPP | −0.18 | 0.06 | 0.05 | 0.09 | −0.08 | 0.08 | −0.07 | 0.10 | 0.14 | 0.08 | 0.14 | 0.11 |
| IL6R | −0.08 | 0.06 | 0.06 | 0.13 | −0.13 | 0.07 | −0.03 | 0.15 | 0.03 | 0.04 | 0.12 | 0.12 |
| PLK1S1 | −0.26 | 0.06 | −0.07 | 0.06 | −0.17 | 0.06 | −0.11 | 0.09 | 0.06 | 0.05 | 0.04 | 0.09 |
| BTNL2 | −0.27 | 0.06 | 0.01 | 0.10 | −0.08 | 0.08 | −0.03 | 0.12 | −0.01 | 0.09 | 0.07 | 0.11 |
| FLOT2 | −0.13 | 0.06 | −0.03 | 0.12 | −0.15 | 0.07 | −0.01 | 0.09 | 0.07 | 0.04 | 0.07 | 0.12 |
| SEMG2 | −0.26 | 0.07 | −0.10 | 0.06 | −0.16 | 0.07 | −0.08 | 0.09 | −0.13 | 0.04 | −0.14 | 0.11 |
| BSN | −0.32 | 0.05 | −0.16 | 0.06 | −0.13 | 0.06 | −0.14 | 0.07 | −0.09 | 0.04 | −0.14 | 0.10 |
| PLD3 | −0.14 | 0.08 | 0.02 | 0.07 | −0.09 | 0.10 | 0.07 | 0.16 | 0.07 | 0.08 | 0.05 | 0.11 |
| PRSS21 | −0.38 | 0.06 | −0.13 | 0.06 | −0.04 | 0.09 | −0.11 | 0.10 | 0.36 | 0.07 | 0.21 | 0.12 |
| ADAR | 0.01 | 0.08 | −0.01 | 0.11 | −0.13 | 0.07 | −0.05 | 0.14 | 0.12 | 0.09 | 0.08 | 0.09 |
| HSPC150 | −0.08 | 0.06 | −0.01 | 0.08 | −0.14 | 0.07 | −0.06 | 0.10 | 0.19 | 0.05 | 0.07 | 0.12 |
| HGD | −0.31 | 0.06 | 0.03 | 0.06 | −0.21 | 0.10 | −0.06 | 0.13 | 0.23 | 0.10 | 0.12 | 0.14 |
| CLIC1 | −0.29 | 0.07 | −0.11 | 0.08 | −0.09 | 0.08 | −0.09 | 0.10 | 0.15 | 0.03 | 0.06 | 0.09 |
| NALP1 | −0.16 | 0.06 | 0.00 | 0.09 | −0.05 | 0.08 | −0.08 | 0.10 | 0.06 | 0.06 | 0.13 | 0.11 |
| LRMP | −0.13 | 0.06 | −0.03 | 0.11 | −0.02 | 0.07 | −0.03 | 0.11 | 0.05 | 0.07 | 0.08 | 0.16 |
| C1ORF91 | 0.06 | 0.06 | 0.02 | 0.06 | −0.15 | 0.06 | 0.04 | 0.09 | 0.08 | 0.05 | −0.09 | 0.08 |
| LOC389727 | 0.02 | 0.09 | 0.00 | 0.09 | −0.13 | 0.09 | −0.03 | 0.13 | 0.28 | 0.09 | 0.20 | 0.12 |
| ACHE | −0.12 | 0.06 | −0.05 | 0.08 | −0.16 | 0.09 | 0.16 | 0.09 | −0.02 | 0.07 | −0.02 | 0.08 |
| BAIAP3 | −0.14 | 0.09 | −0.05 | 0.07 | −0.08 | 0.07 | 0.20 | 0.09 | 0.32 | 0.03 | 0.36 | 0.15 |
| HIRIP3 | −0.15 | 0.06 | −0.09 | 0.08 | −0.17 | 0.07 | −0.09 | 0.11 | −0.01 | 0.04 | 0.03 | 0.11 |
| PPM1B | −0.28 | 0.06 | −0.12 | 0.07 | −0.01 | 0.05 | 0.00 | 0.08 | 0.06 | 0.04 | 0.19 | 0.13 |
| KHSRP | −0.31 | 0.07 | −0.11 | 0.06 | −0.17 | 0.07 | −0.07 | 0.11 | 0.09 | 0.06 | 0.03 | 0.09 |
| ZDHHC9 | −0.25 | 0.07 | −0.05 | 0.11 | −0.19 | 0.06 | −0.03 | 0.12 | 0.16 | 0.06 | 0.10 | 0.09 |
| CD34 | −0.34 | 0.06 | 0.04 | 0.13 | −0.07 | 0.10 | −0.04 | 0.10 | −0.13 | 0.04 | −0.10 | 0.08 |
| TMED1 | −0.19 | 0.07 | −0.02 | 0.09 | −0.12 | 0.10 | −0.03 | 0.09 | 0.14 | 0.04 | 0.04 | 0.16 |
| NCOA3 | −0.15 | 0.08 | −0.07 | 0.07 | −0.10 | 0.06 | 0.00 | 0.16 | 0.18 | 0.07 | 0.08 | 0.09 |
| TCTE3 | −0.15 | 0.07 | 0.00 | 0.09 | −0.14 | 0.08 | −0.04 | 0.09 | 0.24 | 0.05 | 0.19 | 0.12 |
| C10orf92 | −0.17 | 0.08 | 0.07 | 0.09 | −0.07 | 0.07 | 0.12 | 0.13 | 0.37 | 0.06 | 0.28 | 0.18 |
| UBE2L3 | −0.17 | 0.06 | −0.03 | 0.06 | −0.10 | 0.11 | −0.03 | 0.10 | −0.05 | 0.05 | −0.05 | 0.10 |
| NANOS1 | −0.41 | 0.06 | −0.12 | 0.07 | −0.02 | 0.11 | −0.04 | 0.13 | −0.02 | 0.03 | −0.20 | 0.10 |
| LIPC | −0.38 | 0.06 | −0.05 | 0.10 | −0.08 | 0.08 | −0.09 | 0.09 | 0.12 | 0.05 | 0.02 | 0.09 |
| OSTCL | 0.00 | 0.09 | 0.05 | 0.07 | −0.09 | 0.07 | 0.13 | 0.11 | 0.02 | 0.05 | 0.02 | 0.09 |
| GUCA1A | −0.03 | 0.06 | −0.09 | 0.06 | −0.22 | 0.06 | 0.01 | 0.07 | 0.24 | 0.07 | 0.22 | 0.12 |
| EBP | 0.02 | 0.08 | −0.10 | 0.07 | −0.13 | 0.08 | −0.13 | 0.09 | 0.15 | 0.06 | 0.10 | 0.11 |
| SEMA3C | −0.15 | 0.05 | −0.11 | 0.06 | 0.03 | 0.05 | 0.01 | 0.08 | −0.01 | 0.05 | −0.04 | 0.07 |
| KIAA1161 | 0.10 | 0.12 | 0.15 | 0.14 | −0.02 | 0.07 | 0.23 | 0.09 | 0.13 | 0.04 | 0.03 | 0.10 |
| ALK | 0.06 | 0.09 | 0.06 | 0.06 | 0.08 | 0.05 | 0.11 | 0.08 | 0.41 | 0.12 | 0.28 | 0.13 |
| EEF2 | 0.06 | 0.08 | −0.08 | 0.06 | 0.18 | 0.14 | 0.14 | 0.10 | 0.41 | 0.10 | 0.28 | 0.13 |
| COX7C | −0.07 | 0.07 | −0.04 | 0.09 | 0.00 | 0.08 | 0.02 | 0.08 | 0.24 | 0.03 | 0.13 | 0.07 |
| C21orf2 | 0.02 | 0.05 | 0.06 | 0.06 | −0.02 | 0.06 | 0.06 | 0.09 | 0.35 | 0.08 | 0.40 | 0.08 |
| RPSAP32 | 0.05 | 0.08 | −0.07 | 0.07 | −0.15 | 0.08 | 0.03 | 0.08 | 0.32 | 0.11 | 0.11 | 0.09 |
| PDXK | 0.10 | 0.06 | 0.07 | 0.07 | 0.00 | 0.07 | 0.04 | 0.08 | 0.39 | 0.06 | 0.29 | 0.15 |
| MPP6 | 0.08 | 0.06 | −0.09 | 0.07 | 0.08 | 0.09 | −0.04 | 0.10 | 0.11 | 0.06 | 0.24 | 0.15 |
| RAB40A | 0.16 | 0.06 | 0.05 | 0.09 | 0.18 | 0.07 | 0.09 | 0.06 | 0.28 | 0.07 | 0.15 | 0.11 |
| OVCH1 | 0.19 | 0.09 | 0.09 | 0.06 | 0.18 | 0.08 | 0.02 | 0.10 | 0.15 | 0.09 | 0.19 | 0.09 |
| GRLF1 | 0.06 | 0.07 | −0.02 | 0.06 | 0.17 | 0.10 | 0.02 | 0.06 | 0.36 | 0.06 | 0.14 | 0.10 |
| SLC22A1LS | 0.24 | 0.06 | 0.05 | 0.08 | 0.06 | 0.07 | 0.30 | 0.09 | 0.33 | 0.05 | 0.44 | 0.13 |
| LOC442126 | 0.14 | 0.07 | 0.10 | 0.06 | 0.04 | 0.08 | 0.05 | 0.12 | 0.25 | 0.08 | 0.40 | 0.10 |
| DHRSX | 0.09 | 0.09 | −0.02 | 0.06 | 0.08 | 0.06 | 0.00 | 0.08 | 0.08 | 0.05 | 0.07 | 0.07 |
| LOC440056 | 0.23 | 0.14 | 0.05 | 0.09 | −0.02 | 0.09 | 0.08 | 0.10 | 0.34 | 0.14 | 0.37 | 0.10 |
| FBXO42 | 0.24 | 0.08 | 0.05 | 0.07 | 0.12 | 0.08 | 0.09 | 0.09 | 0.16 | 0.04 | 0.22 | 0.14 |
| ALDH7A1 | 0.15 | 0.08 | −0.01 | 0.07 | 0.03 | 0.06 | 0.13 | 0.09 | 0.26 | 0.09 | 0.13 | 0.09 |
| LOC440611 | 0.17 | 0.08 | −0.01 | 0.06 | 0.03 | 0.06 | 0.00 | 0.10 | 0.06 | 0.04 | −0.03 | 0.08 |
| HDLBP | 0.19 | 0.06 | 0.02 | 0.08 | 0.04 | 0.05 | 0.04 | 0.08 | 0.12 | 0.07 | 0.26 | 0.11 |
| LOC441115 | 0.24 | 0.08 | 0.01 | 0.06 | 0.15 | 0.10 | 0.02 | 0.09 | 0.31 | 0.05 | 0.12 | 0.09 |
| ASTL | 0.07 | 0.08 | 0.02 | 0.06 | 0.02 | 0.11 | 0.16 | 0.13 | 0.28 | 0.09 | 0.38 | 0.12 |
| FN3KRP | 0.09 | 0.06 | −0.05 | 0.07 | 0.06 | 0.06 | −0.04 | 0.12 | 0.13 | 0.06 | 0.08 | 0.09 |
| RNF26 | 0.33 | 0.10 | 0.05 | 0.07 | 0.03 | 0.10 | 0.05 | 0.06 | 0.21 | 0.04 | 0.28 | 0.15 |
| PSMC5 | −0.03 | 0.07 | −0.06 | 0.11 | −0.02 | 0.06 | 0.17 | 0.15 | 0.39 | 0.06 | 0.11 | 0.10 |
| TRIP4 | 0.31 | 0.13 | −0.02 | 0.07 | 0.12 | 0.08 | 0.02 | 0.14 | 0.44 | 0.15 | 0.42 | 0.10 |
| SOCS6 | −0.02 | 0.07 | −0.02 | 0.07 | 0.24 | 0.12 | 0.03 | 0.07 | 0.09 | 0.03 | 0.12 | 0.06 |
| PSMD7 | 0.10 | 0.11 | −0.01 | 0.08 | −0.16 | 0.05 | 0.23 | 0.10 | 0.28 | 0.05 | 0.21 | 0.10 |
| COX5B | 0.23 | 0.07 | 0.00 | 0.09 | −0.07 | 0.08 | 0.11 | 0.13 | 0.14 | 0.07 | 0.39 | 0.11 |
| SLC39A5 | 0.14 | 0.05 | −0.04 | 0.07 | 0.12 | 0.09 | 0.12 | 0.13 | 0.42 | 0.16 | 0.42 | 0.13 |
| ZNF878 | 0.16 | 0.10 | 0.10 | 0.08 | 0.12 | 0.08 | 0.17 | 0.17 | 0.27 | 0.06 | 0.19 | 0.11 |
| APAF12 | 0.26 | 0.09 | 0.13 | 0.08 | 0.29 | 0.06 | 0.03 | 0.09 | 0.11 | 0.05 | −0.12 | 0.07 |
| LOC388882 | 0.19 | 0.09 | 0.01 | 0.07 | 0.18 | 0.07 | 0.09 | 0.12 | 0.32 | 0.12 | 0.27 | 0.10 |
| COMMD9 | 0.33 | 0.09 | 0.03 | 0.07 | 0.12 | 0.10 | 0.10 | 0.10 | 0.37 | 0.11 | 0.32 | 0.13 |

Table 4B (in two parts): Alternatively, the siRNA screen hits were evaluated for their ability to
modify the response of A549 to betulinic acid, carboplatin, camptothecin, doxorubicin, etoposide,
mitoxantrone, mytomycin C, oxaliplatin, staurosporine, and thapsigargin. For each hit in each
experimental settings, mean Δ and SEM are reported.
Table 4B (part I)

| | | | | |
|---|---|---|---|---|
| Betulinic acid | Carboplatin | Cisplatin* | Camptothecin | Doxorubicin |

| | Δ** | Δ | Δ | Δ | Δ | Δ | Δ | Δ | Δ | Δ |
|---|---|---|---|---|---|---|---|---|---|---|
| | (Mean) | (SEM) | (Mean) | (SEM) | (Mean) | (SEM) | (Mean) | (SEM) | (Mean) | (SEM) |
| MCL1 | −0.26 | 0.10 | −0.42 | 0.04 | −0.37 | 0.04 | −0.40 | 0.04 | −0.17 | 0.06 |
| TFF1 | −0.09 | 0.06 | −0.27 | 0.05 | −0.36 | 0.05 | −0.16 | 0.07 | −0.21 | 0.05 |
| BCL2L1 | −0.16 | 0.08 | −0.34 | 0.05 | −0.35 | 0.04 | −0.28 | 0.04 | −0.24 | 0.04 |
| RAB3C | −0.07 | 0.07 | −0.25 | 0.05 | −0.33 | 0.06 | −0.12 | 0.05 | −0.17 | 0.04 |
| PPM1J | −0.08 | 0.07 | −0.18 | 0.05 | −0.33 | 0.04 | −0.13 | 0.08 | −0.18 | 0.05 |
| CD151 | −0.09 | 0.10 | −0.33 | 0.04 | −0.32 | 0.12 | −0.30 | 0.04 | −0.03 | 0.03 |
| BMP7 | −0.06 | 0.08 | −0.19 | 0.05 | −0.30 | 0.04 | −0.18 | 0.04 | −0.05 | 0.05 |
| RAC2 | −0.08 | 0.09 | −0.31 | 0.05 | −0.30 | 0.04 | −0.26 | 0.06 | −0.12 | 0.07 |
| WSB2 | −0.04 | 0.07 | −0.36 | 0.04 | −0.30 | 0.05 | −0.30 | 0.04 | −0.18 | 0.06 |
| LOC390533 | −0.10 | 0.10 | −0.30 | 0.04 | −0.29 | 0.09 | −0.24 | 0.06 | −0.09 | 0.06 |
| CYP11B2 | −0.02 | 0.11 | −0.23 | 0.05 | −0.28 | 0.05 | −0.14 | 0.07 | −0.10 | 0.05 |
| RRAD | −0.14 | 0.08 | −0.23 | 0.08 | −0.28 | 0.05 | −0.22 | 0.06 | 0.02 | 0.06 |
| MAT2A | 0.03 | 0.09 | −0.11 | 0.04 | −0.27 | 0.05 | 0.04 | 0.09 | −0.08 | 0.04 |
| USP8 | −0.14 | 0.07 | −0.19 | 0.05 | −0.27 | 0.04 | −0.20 | 0.06 | −0.25 | 0.04 |
| ITGB6 | −0.21 | 0.06 | −0.32 | 0.03 | −0.27 | 0.05 | −0.35 | 0.05 | −0.03 | 0.03 |
| DEFB4 | −0.07 | 0.09 | −0.25 | 0.05 | −0.27 | 0.04 | −0.21 | 0.06 | −0.02 | 0.04 |
| NCKAP1 | −0.12 | 0.08 | −0.32 | 0.04 | −0.27 | 0.05 | −0.30 | 0.05 | −0.04 | 0.07 |
| IAPP | −0.05 | 0.08 | −0.26 | 0.04 | −0.26 | 0.04 | −0.24 | 0.04 | −0.05 | 0.08 |
| IL6R | −0.06 | 0.06 | −0.11 | 0.06 | −0.25 | 0.04 | −0.05 | 0.06 | 0.04 | 0.05 |
| PLK1S1 | −0.05 | 0.08 | −0.11 | 0.07 | −0.25 | 0.05 | −0.11 | 0.08 | −0.09 | 0.04 |
| BTNL2 | −0.03 | 0.09 | −0.18 | 0.05 | −0.25 | 0.05 | −0.06 | 0.05 | −0.01 | 0.06 |
| FLOT2 | −0.04 | 0.06 | −0.15 | 0.05 | −0.25 | 0.05 | −0.19 | 0.05 | −0.10 | 0.04 |
| SEMG2 | −0.06 | 0.06 | −0.17 | 0.04 | −0.24 | 0.05 | −0.13 | 0.04 | −0.06 | 0.04 |
| BSN | −0.04 | 0.07 | −0.20 | 0.04 | −0.24 | 0.05 | −0.23 | 0.06 | −0.03 | 0.07 |
| PLD3 | −0.08 | 0.06 | −0.28 | 0.05 | −0.24 | 0.05 | −0.33 | 0.04 | −0.12 | 0.05 |
| PRSS21 | −0.08 | 0.07 | −0.13 | 0.06 | −0.24 | 0.04 | −0.19 | 0.06 | −0.05 | 0.07 |
| ADAR | −0.16 | 0.06 | −0.18 | 0.06 | −0.24 | 0.04 | −0.13 | 0.07 | 0.03 | 0.04 |
| HSPC150 | −0.06 | 0.08 | −0.27 | 0.04 | −0.24 | 0.05 | −0.25 | 0.04 | −0.05 | 0.05 |
| HGD | −0.14 | 0.05 | −0.31 | 0.05 | −0.24 | 0.05 | −0.35 | 0.05 | −0.22 | 0.03 |
| CLIC1 | −0.08 | 0.08 | −0.13 | 0.05 | −0.23 | 0.04 | 0.01 | 0.06 | −0.07 | 0.03 |
| NALP1 | −0.07 | 0.07 | −0.11 | 0.04 | −0.22 | 0.04 | −0.10 | 0.04 | −0.16 | 0.03 |
| LRMP | −0.08 | 0.07 | −0.13 | 0.05 | −0.22 | 0.05 | −0.01 | 0.06 | 0.04 | 0.04 |
| C1ORF91 | −0.07 | 0.06 | −0.17 | 0.04 | −0.22 | 0.05 | −0.14 | 0.04 | −0.07 | 0.03 |
| LOC389727 | −0.09 | 0.06 | −0.18 | 0.04 | −0.21 | 0.05 | −0.14 | 0.04 | −0.12 | 0.03 |
| ACHE | −0.06 | 0.10 | −0.16 | 0.06 | −0.21 | 0.05 | −0.12 | 0.05 | 0.04 | 0.06 |
| BAIAP3 | 0.01 | 0.09 | −0.21 | 0.05 | −0.21 | 0.06 | −0.20 | 0.08 | −0.07 | 0.05 |
| HIRIP3 | −0.06 | 0.07 | −0.19 | 0.03 | −0.21 | 0.05 | −0.12 | 0.06 | −0.06 | 0.05 |
| PPM1B | 0.05 | 0.11 | −0.17 | 0.04 | −0.21 | 0.06 | −0.13 | 0.08 | −0.01 | 0.07 |
| KHSRP | −0.08 | 0.08 | −0.26 | 0.06 | −0.21 | 0.05 | −0.13 | 0.05 | −0.11 | 0.05 |
| ZDHHC9 | −0.05 | 0.08 | −0.15 | 0.04 | −0.20 | 0.05 | −0.12 | 0.07 | 0.01 | 0.05 |
| CD34 | −0.03 | 0.08 | −0.19 | 0.05 | −0.20 | 0.05 | −0.10 | 0.04 | −0.02 | 0.04 |
| TMED1 | −0.08 | 0.07 | −0.27 | 0.05 | −0.19 | 0.04 | −0.24 | 0.04 | 0.06 | 0.03 |
| NCOA3 | −0.06 | 0.08 | −0.33 | 0.05 | −0.19 | 0.04 | −0.32 | 0.07 | 0.06 | 0.06 |
| TCTE3 | −0.05 | 0.09 | −0.16 | 0.05 | −0.19 | 0.05 | −0.08 | 0.07 | 0.00 | 0.03 |
| C10orf92 | −0.17 | 0.08 | −0.19 | 0.06 | −0.18 | 0.09 | −0.36 | 0.05 | −0.12 | 0.07 |
| UBE2L3 | −0.03 | 0.11 | −0.27 | 0.04 | −0.17 | 0.04 | −0.23 | 0.05 | −0.04 | 0.05 |
| NANOS1 | 0.02 | 0.09 | −0.15 | 0.05 | −0.17 | 0.06 | −0.11 | 0.06 | −0.05 | 0.05 |
| LIPC | −0.14 | 0.07 | −0.14 | 0.04 | −0.16 | 0.05 | −0.09 | 0.06 | −0.19 | 0.04 |
| OSTCL | −0.03 | 0.09 | −0.15 | 0.04 | −0.16 | 0.07 | −0.21 | 0.06 | 0.01 | 0.04 |
| GUCA1A | −0.04 | 0.09 | −0.13 | 0.05 | −0.14 | 0.07 | −0.28 | 0.06 | −0.04 | 0.05 |
| EBP | 0.00 | 0.07 | −0.19 | 0.06 | −0.13 | 0.06 | −0.19 | 0.06 | −0.14 | 0.03 |
| SEMA3C | 0.08 | 0.07 | −0.10 | 0.04 | −0.11 | 0.06 | −0.18 | 0.08 | 0.01 | 0.06 |
| KIAA1161 | 0.06 | 0.08 | −0.20 | 0.06 | −0.02 | 0.06 | −0.19 | 0.06 | 0.14 | 0.07 |
| ALK | 0.01 | 0.07 | −0.08 | 0.05 | 0.01 | 0.05 | −0.15 | 0.11 | 0.15 | 0.07 |
| EEF2 | −0.03 | 0.10 | −0.01 | 0.07 | 0.02 | 0.06 | 0.02 | 0.13 | 0.08 | 0.05 |
| COX7C | 0.07 | 0.10 | 0.07 | 0.05 | 0.05 | 0.06 | −0.01 | 0.09 | 0.26 | 0.06 |
| C21orf2 | 0.00 | 0.07 | −0.17 | 0.04 | 0.06 | 0.06 | −0.06 | 0.09 | 0.14 | 0.07 |
| RPSAP32 | 0.01 | 0.09 | −0.02 | 0.04 | 0.07 | 0.07 | −0.02 | 0.06 | −0.05 | 0.05 |
| PDXK | 0.03 | 0.06 | 0.05 | 0.06 | 0.07 | 0.07 | −0.07 | 0.07 | 0.01 | 0.04 |
| MPP6 | 0.13 | 0.07 | 0.04 | 0.05 | 0.08 | 0.08 | 0.01 | 0.08 | 0.04 | 0.06 |
| RAB40A | 0.00 | 0.07 | −0.03 | 0.04 | 0.09 | 0.07 | −0.13 | 0.08 | 0.13 | 0.06 |
| OVCH1 | 0.01 | 0.07 | −0.11 | 0.04 | 0.11 | 0.05 | −0.22 | 0.09 | 0.04 | 0.05 |
| GRLF1 | 0.09 | 0.07 | −0.05 | 0.04 | 0.11 | 0.07 | 0.02 | 0.08 | 0.16 | 0.06 |
| SLC22A1LS | −0.02 | 0.10 | 0.00 | 0.05 | 0.11 | 0.08 | −0.05 | 0.06 | 0.12 | 0.08 |
| LOC442126 | 0.00 | 0.06 | −0.06 | 0.04 | 0.12 | 0.07 | −0.21 | 0.07 | 0.19 | 0.09 |
| DHRSX | 0.05 | 0.07 | 0.05 | 0.04 | 0.13 | 0.07 | −0.03 | 0.07 | 0.12 | 0.05 |
| LOC440056 | −0.10 | 0.06 | 0.03 | 0.04 | 0.14 | 0.06 | −0.07 | 0.08 | 0.18 | 0.05 |
| FBXO42 | 0.06 | 0.06 | −0.09 | 0.04 | 0.14 | 0.06 | −0.06 | 0.06 | 0.01 | 0.05 |
| ALDH7A1 | 0.06 | 0.07 | −0.02 | 0.05 | 0.14 | 0.06 | −0.15 | 0.07 | 0.16 | 0.05 |
| LOC440611 | 0.13 | 0.07 | 0.00 | 0.05 | 0.14 | 0.06 | −0.08 | 0.06 | 0.02 | 0.05 |
| HDLBP | 0.04 | 0.08 | 0.10 | 0.05 | 0.15 | 0.06 | 0.03 | 0.07 | 0.06 | 0.06 |
| LOC441115 | −0.01 | 0.08 | −0.09 | 0.06 | 0.16 | 0.07 | −0.22 | 0.07 | 0.09 | 0.07 |
| ASTL | −0.04 | 0.08 | 0.04 | 0.06 | 0.17 | 0.07 | −0.10 | 0.06 | 0.01 | 0.05 |
| FN3KRP | 0.11 | 0.09 | −0.06 | 0.04 | 0.17 | 0.07 | −0.08 | 0.08 | 0.10 | 0.06 |
| RNF26 | −0.01 | 0.07 | −0.04 | 0.05 | 0.18 | 0.07 | −0.14 | 0.06 | 0.05 | 0.07 |
| PSMC5 | −0.05 | 0.08 | −0.03 | 0.04 | 0.18 | 0.09 | −0.02 | 0.09 | 0.02 | 0.06 |
| TRIP4 | 0.03 | 0.08 | 0.04 | 0.06 | 0.20 | 0.07 | 0.08 | 0.08 | 0.03 | 0.07 |
| SOCS6 | 0.01 | 0.07 | −0.01 | 0.05 | 0.21 | 0.08 | −0.06 | 0.09 | 0.13 | 0.05 |
| PSMD7 | 0.00 | 0.07 | 0.07 | 0.04 | 0.22 | 0.05 | 0.06 | 0.08 | 0.11 | 0.06 |
| COX5B | 0.07 | 0.07 | 0.08 | 0.06 | 0.23 | 0.05 | −0.02 | 0.08 | 0.12 | 0.06 |
| SLC39A5 | 0.09 | 0.07 | 0.09 | 0.05 | 0.23 | 0.07 | 0.11 | 0.08 | 0.19 | 0.06 |
| ZNF878 | 0.14 | 0.09 | 0.12 | 0.05 | 0.25 | 0.08 | 0.01 | 0.08 | −0.01 | 0.05 |
| APAF12 | 0.04 | 0.06 | −0.06 | 0.06 | 0.25 | 0.07 | −0.10 | 0.06 | 0.06 | 0.04 |
| LOC388882 | −0.04 | 0.06 | 0.01 | 0.06 | 0.26 | 0.06 | −0.05 | 0.07 | 0.11 | 0.07 |
| COMMD9 | 0.08 | 0.10 | 0.06 | 0.04 | 0.35 | 0.09 | −0.02 | 0.07 | 0.08 | 0.04 |

Table 4B (part II):

| | Etoposide | | Mitoxantrone | | Mytomycin C | | Oxaliplatin | | Staurosporine | | Thapsigargin | |
|---|---|---|---|---|---|---|---|---|---|---|---|---|
| | Δ | Δ | Δ | Δ | Δ | Δ | Δ | Δ | Δ | Δ | Δ | Δ |
| | (Mean) | (SEM) | (Mean) | (SEM) | (Mean) | (SEM) | (Mean) | (SEM) | (Mean) | (SEM) | (Mean) | (SEM) |
| MCL1 | −0.35 | 0.06 | −0.18 | 0.05 | −0.21 | 0.08 | −0.24 | 0.05 | −0.25 | 0.05 | −0.11 | 0.07 |
| TFF1 | −0.05 | 0.06 | −0.20 | 0.04 | −0.23 | 0.08 | −0.11 | 0.05 | −0.07 | 0.07 | −0.08 | 0.06 |
| BCL2L1 | −0.12 | 0.07 | −0.17 | 0.03 | −0.22 | 0.10 | −0.25 | 0.05 | −0.20 | 0.06 | −0.06 | 0.03 |
| RAB3C | −0.06 | 0.08 | −0.09 | 0.04 | −0.20 | 0.11 | −0.11 | 0.06 | −0.16 | 0.05 | 0.06 | 0.08 |
| PPM1J | −0.13 | 0.09 | −0.12 | 0.04 | −0.21 | 0.08 | −0.02 | 0.05 | −0.18 | 0.05 | 0.15 | 0.06 |
| CD151 | −0.03 | 0.08 | −0.03 | 0.07 | −0.08 | 0.09 | −0.08 | 0.06 | −0.17 | 0.05 | 0.02 | 0.08 |
| BMP7 | −0.06 | 0.07 | −0.10 | 0.06 | 0.00 | 0.09 | 0.01 | 0.07 | −0.17 | 0.05 | 0.16 | 0.03 |
| RAC2 | −0.06 | 0.10 | −0.12 | 0.03 | −0.24 | 0.09 | −0.10 | 0.06 | −0.07 | 0.06 | −0.02 | 0.07 |
| WSB2 | −0.04 | 0.13 | −0.18 | 0.04 | −0.27 | 0.10 | −0.25 | 0.05 | 0.04 | 0.07 | −0.10 | 0.05 |
| LOC390533 | −0.12 | 0.08 | −0.14 | 0.05 | −0.08 | 0.10 | −0.09 | 0.06 | −0.13 | 0.05 | −0.02 | 0.09 |
| CYP11B2 | 0.00 | 0.06 | −0.16 | 0.04 | −0.04 | 0.13 | −0.07 | 0.05 | −0.17 | 0.05 | 0.20 | 0.06 |
| RRAD | −0.09 | 0.06 | −0.18 | 0.03 | −0.11 | 0.09 | −0.07 | 0.05 | 0.05 | 0.07 | −0.10 | 0.05 |
| MAT2A | 0.10 | 0.08 | −0.03 | 0.05 | 0.02 | 0.09 | 0.14 | 0.05 | −0.05 | 0.06 | 0.17 | 0.05 |
| USP8 | −0.13 | 0.07 | −0.25 | 0.03 | −0.24 | 0.19 | −0.06 | 0.05 | −0.16 | 0.06 | −0.03 | 0.07 |
| ITGB6 | −0.10 | 0.09 | −0.07 | 0.07 | −0.19 | 0.09 | −0.09 | 0.05 | −0.09 | 0.05 | −0.06 | 0.06 |
| DEFB4 | −0.18 | 0.06 | −0.06 | 0.04 | −0.07 | 0.09 | −0.05 | 0.05 | −0.17 | 0.05 | 0.29 | 0.09 |
| NCKAP1 | −0.15 | 0.09 | −0.06 | 0.05 | −0.04 | 0.09 | −0.08 | 0.06 | −0.06 | 0.06 | −0.05 | 0.09 |
| IAPP | −0.12 | 0.09 | −0.05 | 0.04 | −0.10 | 0.08 | −0.05 | 0.06 | −0.15 | 0.04 | 0.08 | 0.03 |
| IL6R | −0.12 | 0.07 | 0.08 | 0.04 | −0.01 | 0.09 | 0.02 | 0.05 | 0.00 | 0.06 | −0.02 | 0.06 |
| PLK1S1 | −0.11 | 0.06 | −0.13 | 0.03 | −0.12 | 0.15 | 0.03 | 0.05 | −0.21 | 0.05 | 0.01 | 0.03 |
| BTNL2 | −0.04 | 0.08 | −0.06 | 0.04 | 0.01 | 0.09 | 0.03 | 0.05 | 0.13 | 0.07 | 0.04 | 0.06 |
| FLOT2 | −0.07 | 0.08 | −0.13 | 0.04 | 0.06 | 0.10 | −0.04 | 0.06 | −0.18 | 0.06 | 0.24 | 0.04 |
| SEMG2 | −0.05 | 0.06 | −0.01 | 0.03 | −0.02 | 0.09 | 0.00 | 0.05 | −0.03 | 0.06 | 0.30 | 0.03 |
| BSN | −0.19 | 0.08 | −0.06 | 0.05 | −0.13 | 0.06 | −0.05 | 0.05 | −0.11 | 0.05 | 0.04 | 0.06 |
| PLD3 | −0.14 | 0.07 | −0.17 | 0.05 | −0.14 | 0.11 | −0.19 | 0.05 | −0.13 | 0.05 | −0.08 | 0.04 |
| PRSS21 | −0.02 | 0.07 | −0.12 | 0.03 | −0.05 | 0.17 | −0.02 | 0.06 | 0.01 | 0.05 | −0.06 | 0.05 |
| ADAR | −0.15 | 0.08 | −0.11 | 0.03 | −0.07 | 0.10 | −0.01 | 0.06 | 0.01 | 0.05 | 0.07 | 0.05 |
| HSPC150 | −0.10 | 0.09 | −0.14 | 0.04 | 0.00 | 0.10 | −0.10 | 0.05 | −0.14 | 0.05 | 0.20 | 0.07 |
| HGD | −0.25 | 0.08 | −0.23 | 0.04 | −0.17 | 0.09 | −0.11 | 0.05 | −0.20 | 0.05 | −0.18 | 0.04 |
| CLIC1 | −0.11 | 0.07 | −0.07 | 0.04 | 0.06 | 0.15 | 0.11 | 0.06 | 0.04 | 0.07 | −0.10 | 0.05 |
| NALP1 | −0.06 | 0.06 | −0.11 | 0.04 | 0.11 | 0.10 | 0.01 | 0.05 | 0.00 | 0.05 | 0.23 | 0.05 |
| LRMP | −0.11 | 0.07 | 0.04 | 0.05 | 0.01 | 0.09 | 0.06 | 0.07 | −0.08 | 0.05 | 0.34 | 0.05 |
| C1ORF91 | −0.08 | 0.07 | −0.04 | 0.03 | 0.01 | 0.09 | −0.07 | 0.05 | −0.13 | 0.06 | 0.03 | 0.04 |
| LOC389727 | −0.14 | 0.08 | −0.11 | 0.04 | −0.05 | 0.09 | −0.01 | 0.05 | −0.01 | 0.06 | 0.07 | 0.06 |
| ACHE | −0.03 | 0.07 | −0.03 | 0.05 | −0.10 | 0.13 | −0.01 | 0.05 | −0.10 | 0.06 | −0.03 | 0.06 |
| BAIAP3 | −0.06 | 0.09 | −0.03 | 0.05 | −0.10 | 0.07 | −0.07 | 0.05 | 0.02 | 0.07 | 0.01 | 0.05 |
| HIRIP3 | −0.13 | 0.09 | −0.08 | 0.04 | −0.13 | 0.12 | −0.01 | 0.06 | −0.10 | 0.06 | 0.25 | 0.04 |
| PPM1B | 0.13 | 0.12 | −0.05 | 0.05 | −0.01 | 0.07 | 0.03 | 0.06 | −0.02 | 0.05 | 0.14 | 0.06 |
| KHSRP | −0.15 | 0.08 | −0.13 | 0.05 | −0.06 | 0.10 | −0.15 | 0.05 | −0.10 | 0.07 | 0.14 | 0.05 |
| ZDHHC9 | −0.10 | 0.07 | −0.05 | 0.05 | 0.05 | 0.10 | 0.04 | 0.08 | −0.16 | 0.06 | 0.17 | 0.11 |
| CD34 | −0.08 | 0.05 | −0.04 | 0.06 | −0.04 | 0.09 | −0.01 | 0.05 | −0.01 | 0.06 | 0.13 | 0.04 |
| TMED1 | −0.07 | 0.07 | −0.03 | 0.04 | −0.12 | 0.08 | −0.05 | 0.07 | 0.11 | 0.07 | −0.10 | 0.05 |
| NCOA3 | −0.09 | 0.07 | 0.03 | 0.05 | −0.04 | 0.10 | −0.21 | 0.06 | −0.17 | 0.05 | 0.06 | 0.06 |
| TCTE3 | −0.03 | 0.06 | 0.01 | 0.03 | −0.05 | 0.09 | 0.01 | 0.06 | −0.10 | 0.05 | 0.25 | 0.09 |
| C10orf92 | −0.09 | 0.08 | −0.14 | 0.03 | −0.15 | 0.11 | −0.14 | 0.07 | 0.09 | 0.08 | −0.05 | 0.08 |
| UBE2L3 | −0.13 | 0.06 | −0.08 | 0.05 | −0.03 | 0.09 | −0.01 | 0.05 | −0.05 | 0.06 | 0.08 | 0.08 |
| NANOS1 | −0.02 | 0.06 | −0.09 | 0.04 | −0.08 | 0.18 | −0.06 | 0.06 | −0.13 | 0.05 | 0.18 | 0.11 |
| LIPC | −0.06 | 0.08 | −0.18 | 0.04 | −0.04 | 0.08 | 0.02 | 0.05 | −0.12 | 0.06 | −0.02 | 0.05 |
| OSTCL | −0.08 | 0.08 | −0.09 | 0.04 | 0.02 | 0.08 | 0.02 | 0.05 | −0.09 | 0.04 | 0.18 | 0.05 |
| GUCA1A | 0.08 | 0.11 | −0.05 | 0.07 | 0.01 | 0.07 | 0.05 | 0.06 | −0.02 | 0.04 | −0.04 | 0.05 |
| EBP | −0.07 | 0.07 | −0.07 | 0.04 | 0.02 | 0.11 | −0.06 | 0.05 | −0.13 | 0.06 | 0.23 | 0.06 |
| SEMA3C | −0.05 | 0.09 | 0.05 | 0.05 | −0.07 | 0.06 | −0.01 | 0.05 | −0.12 | 0.04 | 0.09 | 0.05 |
| KIAA1161 | −0.04 | 0.09 | 0.05 | 0.04 | −0.13 | 0.09 | −0.12 | 0.06 | −0.09 | 0.05 | 0.11 | 0.06 |
| ALK | 0.07 | 0.11 | 0.17 | 0.10 | −0.11 | 0.10 | 0.07 | 0.07 | 0.06 | 0.05 | 0.07 | 0.09 |
| EEF2 | 0.20 | 0.16 | 0.07 | 0.09 | −0.04 | 0.11 | 0.07 | 0.07 | 0.04 | 0.05 | 0.02 | 0.06 |
| COX7C | 0.18 | 0.11 | 0.12 | 0.09 | 0.04 | 0.07 | 0.11 | 0.06 | 0.04 | 0.04 | 0.13 | 0.07 |
| C21orf2 | −0.05 | 0.10 | 0.13 | 0.09 | −0.06 | 0.07 | −0.01 | 0.06 | 0.14 | 0.04 | 0.15 | 0.04 |
| RPSAP32 | −0.03 | 0.11 | −0.04 | 0.07 | −0.16 | 0.14 | −0.05 | 0.06 | −0.14 | 0.05 | 0.22 | 0.07 |
| PDXK | −0.03 | 0.09 | 0.11 | 0.08 | 0.02 | 0.08 | 0.09 | 0.07 | 0.01 | 0.05 | 0.24 | 0.06 |
| MPP6 | −0.02 | 0.10 | 0.10 | 0.07 | 0.00 | 0.06 | 0.04 | 0.06 | −0.10 | 0.04 | 0.23 | 0.07 |
| RAB40A | −0.06 | 0.10 | 0.06 | 0.07 | −0.10 | 0.07 | 0.05 | 0.06 | 0.04 | 0.05 | 0.18 | 0.05 |
| OVCH1 | −0.10 | 0.10 | 0.20 | 0.07 | 0.19 | 0.07 | 0.01 | 0.05 | −0.05 | 0.05 | 0.19 | 0.10 |
| GRLF1 | 0.07 | 0.09 | 0.22 | 0.08 | 0.10 | 0.07 | 0.12 | 0.06 | −0.11 | 0.04 | 0.21 | 0.05 |
| SLC22A1LS | 0.06 | 0.12 | −0.03 | 0.06 | −0.05 | 0.06 | 0.02 | 0.07 | −0.13 | 0.03 | −0.01 | 0.04 |
| LOC442126 | −0.07 | 0.10 | 0.15 | 0.07 | −0.04 | 0.06 | 0.05 | 0.05 | 0.15 | 0.04 | 0.16 | 0.07 |
| DHRSX | 0.01 | 0.11 | 0.15 | 0.08 | 0.05 | 0.06 | 0.09 | 0.06 | −0.02 | 0.04 | 0.24 | 0.08 |
| LOC440056 | 0.03 | 0.15 | 0.12 | 0.07 | −0.01 | 0.07 | −0.03 | 0.05 | 0.19 | 0.05 | 0.19 | 0.08 |
| FBXO42 | −0.01 | 0.10 | 0.18 | 0.07 | −0.03 | 0.08 | −0.02 | 0.06 | −0.09 | 0.04 | 0.04 | 0.04 |
| ALDH7A1 | 0.06 | 0.10 | 0.05 | 0.07 | −0.01 | 0.06 | −0.01 | 0.05 | −0.19 | 0.04 | 0.23 | 0.06 |
| LOC440611 | 0.09 | 0.09 | 0.12 | 0.07 | 0.04 | 0.07 | 0.01 | 0.06 | 0.04 | 0.04 | 0.25 | 0.05 |
| HDLBP | 0.11 | 0.11 | 0.18 | 0.07 | −0.02 | 0.09 | 0.11 | 0.07 | 0.06 | 0.04 | 0.19 | 0.08 |
| LOC441115 | 0.05 | 0.16 | 0.24 | 0.11 | 0.00 | 0.06 | −0.05 | 0.06 | −0.07 | 0.05 | 0.09 | 0.06 |
| ASTL | 0.03 | 0.10 | 0.00 | 0.07 | −0.02 | 0.07 | 0.10 | 0.05 | −0.03 | 0.05 | 0.07 | 0.07 |
| FN3KRP | 0.06 | 0.12 | 0.18 | 0.09 | 0.02 | 0.07 | −0.02 | 0.12 | 0.08 | 0.11 | 0.21 | 0.07 |
| RNF26 | −0.09 | 0.10 | −0.04 | 0.05 | 0.04 | 0.07 | −0.03 | 0.06 | −0.09 | 0.04 | 0.19 | 0.06 |
| PSMC5 | 0.04 | 0.12 | −0.11 | 0.05 | −0.01 | 0.09 | 0.10 | 0.05 | −0.18 | 0.04 | 0.01 | 0.06 |
| TRIP4 | 0.04 | 0.13 | 0.02 | 0.06 | 0.12 | 0.08 | 0.01 | 0.05 | −0.02 | 0.04 | 0.24 | 0.08 |
| SOCS6 | 0.00 | 0.09 | 0.25 | 0.13 | −0.01 | 0.06 | −0.04 | 0.06 | 0.00 | 0.05 | 0.18 | 0.09 |
| PSMD7 | 0.09 | 0.11 | 0.13 | 0.07 | 0.04 | 0.06 | 0.12 | 0.05 | −0.02 | 0.03 | 0.29 | 0.06 |
| COX5B | 0.11 | 0.09 | 0.06 | 0.08 | 0.10 | 0.07 | 0.12 | 0.07 | 0.12 | 0.05 | 0.02 | 0.05 |
| SLC39A5 | 0.11 | 0.10 | 0.13 | 0.08 | 0.01 | 0.07 | 0.13 | 0.07 | 0.15 | 0.03 | 0.22 | 0.06 |
| ZNF878 | 0.09 | 0.10 | 0.06 | 0.06 | 0.04 | 0.09 | 0.14 | 0.05 | −0.11 | 0.03 | 0.28 | 0.06 |
| APAF12 | −0.03 | 0.10 | 0.06 | 0.04 | −0.02 | 0.07 | 0.01 | 0.07 | −0.08 | 0.03 | 0.17 | 0.05 |
| LOC388882 | 0.00 | 0.11 | 0.13 | 0.08 | 0.16 | 0.07 | 0.03 | 0.06 | −0.12 | 0.03 | 0.03 | 0.06 |
| COMMD9 | 0.11 | 0.11 | 0.07 | 0.07 | 0.00 | 0.07 | 0.02 | 0.06 | −0.04 | 0.05 | 0.09 | 0.06 |

* = reference profile
**= upon background subtraction and inter-plate normalization, Δ = relative survival of siRNA-transfected cells (WST-1 signal from CDDP treated, siRNA-transfected cells/WST-1 signal from untreated, siRNA-transfected cells) - relative survival of cells transfected with a control siRNA (siUNR) (WST-1 signal from CDDP treated, siUNR-transfected cells/WST-1 signal from untreated, siUNR-transfected cells). Negative and positive Δ values signify chemosensitization and cytoprotection, respectively.
SEM = standard error of the mean

### Epistatic Screening of CDDP Response Modifiers.

To gain further insights into the functional relationship between the CRMs identified in this screen, inventors performed a systematic epistatic analysis. For this, they selected the most efficient siRNAs corresponding to each of the 85 hits (FIG. 1) and to 7 additional genes (BAK1, BAX, BCL2, CASP2, STAT3, TP53, VDAC1) identified as possible CRMs in the past.

A549 cells were transfected with 92×92 couples of siRNAs, treated with CDDP, and characterized for the combined effects of the siRNAs on WST-1 conversion. The resultant matrix of epistatic effects (FIG. 4A) was subjected to unsupervised hierarchical clustering (following the Ward method and the Pearson correlation), leading to the identification of four separate clusters (Table 5). Within each cluster, combined knockdown of two transcripts mostly failed to yield significant epistatic interactions, while the knockdown of transcript belonging to distinct clusters caused epistasis. Some of these results were expected (such as the classification of the anti-apoptotic proteins BCL2, BCL-X_{L} and MCL1 in cluster 1, that of the COX subunits Vb and VIId in cluster 3, and that of the pro-apoptotic proteins BAX, BAK1, APAF1, TP53, VDAC1, CASP2, the BCL2 interactor NLRP1, and two proteasome subunits in cluster 4), while others were not (such as the presence of IL6R, STAT3 and CLIC1 in cluster 1).

These results were validated using siRNAs (FIG. 4B, 4D, 4F) and pharmacological inhibitors (FIG. 4C, 4E, 4G), followed by DiOC₆(3)/PI co-staining as an alternative readout. Thus the combined depletion/inhibition of ZDHHC9 (which is inhibited by 2-bromopalmitate) and IL6R (which can be blocked by an extracellular antibody), both belonging to cluster 1, yields no significant epistatic interaction (FIG. 4B, 4C). In contrast, simultaneous depletion/inhibition of IL6R (in cluster 1) and LIPC (which can be inhibited by orlistat and belonged to cluster 2) advantageously synergistically sensitized to CDDP-induced killing (FIG. 4C, 4D). Moreover, concomitant knockdown or inhibition of COX (in cluster 3) with sodium azide and TP53 (in cluster 4) with cyclic pifithrin-α yielded synergistic protection against CDDP (FIG. 4F, 4G). Altogether, these results demonstrate that (at least) four different signalling modules determine CDDP responses.

TABLE 5
Epistatic clustering.
Epistatic analyses upon co-transfection of the 85 siRNA screen
allowed for the identification of 4 clusters of cisplatin
response modifiers (CRMs). For each the hit,
accession number and cluster are reported. See also FIGS. 4.

| Name | Accession number | Cluster |
|---|---|---|
| ACHE | NM_000665 | 1 |
| ADAR | NM_001111 | 1 |
| ALDH7A1 | NM_001182 | 4 |
| ALK | NM_004304 | 3 |
| APAF1 | NM_001160 | 4 |
| ASTL | NM_001002036 | 4 |
| BAIAP3 | NM_003933 | 1 |
| BAK1 | NM_001188 | 4 |
| BAX | NM_004324 | 4 |
| BCL2 | NM_000657/NM_000633 | 1 |
| BCL2L1 | NM_001191 | 1 |
| BMP7 | NM_001719 | 2 |
| BSN | NM_003458 | 1 |
| BTNL2 | NM_019602 | 1 |
| C10ORF92 | NM_017609 | 1 |
| C1ORF91 | NM_019118 | 1 |
| C21ORF2 | NM_004928 | 4 |
| CASP2 | NM_032982/NM_032983 | 4 |
| CD151 | NM_004357 | 1 |
| CD34 | NM_001773 | 1 |
| CLIC1 | NM_001288 | 1 |
| COMMD9 | NM_014186 | 4 |
| COX5B | NM_001862 | 3 |
| COX7C | NM_001867 | 3 |
| CYP11B2 | NM_000498 | 1 |
| DEFB4 | NM_004942 | 1 |
| DHRSX | NM_145177 | 3 |
| EBP | NM_006579 | 1 |
| EEF2 | NM_001961 | 3 |
| FBXO42 | NM_018994 | 4 |
| FLOT2 | NM_004475 | 1 |
| FN3KRP | NM_024619 | 4 |
| GRLF1 | NM_004491 | 4 |
| GUCA1A | NM_000409 | 3 |
| HDLBP | NM_005336 | 4 |
| HGD | NM_000187 | 2 |
| HIRIP3 | NM_003609 | 1 |
| IAPP | NM_000415 | 2 |
| IL6R | NM_000565 | 1 |
| ITGB6 | NM_000888 | 1 |
| KHSRP | NM_003685 | 1 |
| KIA1161 | NM_020702 | 4 |
| LIPC | NM_000236 | 2 |
| LOC388882 | XR_042117 | 4 |
| LOC389727 | Pseudogene | 2 |
| LOC390533 | Discontinued | 2 |
| RPSAP32 | Pseudogene | 4 |
| LOC440056 | Discontinued | 4 |
| LOC440611 | Discontinued | 3 |
| LOC441115 | Discontinued | 4 |
| LOC442126 | Discontinued | 4 |
| LRMP | NM_006152 | 2 |
| MAT2A | NM_005911 | 1 |
| MCL1 | NM_021960 | 1 |
| MPP6 | NM_016447 | 4 |
| NANOS1 | NM_199461 | 1 |
| NCKAP1 | NM_013436 | 1 |
| NCOA3 | NM_006534 | 1 |
| NLRP1 | NM_014922 | 4 |
| OSTCL | NM_145303 | 1 |
| OVCH1 | NM_183378 | 4 |
| PDXK | NM_003681 | 4 |
| PLD3 | NM_012268 | 1 |
| PLK1S1 | NM_018474 | 1 |
| PPM1B | NM_002706 | 1 |
| PPM1J | NM_005167 | 1 |
| PRSS21 | NM_006799 | 2 |
| PSMC5 | NM_002805 | 4 |
| PSMD7 | NM_002811 | 4 |
| RAB3C | NM_138453 | 2 |
| RAB40A | NM_080879 | 4 |
| RAC2 | NM_002872 | 1 |
| RNF26 | NM_032015 | 4 |
| RRAD | NM_004165 | 1 |
| SEMA3C | NM_006379 | 2 |
| SEMG2 | NM_003008 | 1 |
| SLC22A18AS | NM_007105 | 4 |
| SLC39A5 | NM_173596 | 4 |
| SOCS6 | NM_004232 | 4 |
| STAT3 | NM_003150 | 1 |
| TCTE3 | NM_174910 | 2 |
| TFF1 | NM_003225 | 1 |
| TMED1 | NM_006858 | 4 |
| TP53 | NM_000546 | 4 |
| TRIP4 | NM_016213 | 4 |
| UBE2L3 | NM_003347 | 1 |
| UBE2T | NM_014176 | 1 |
| USP8 | NM_005154 | 1 |
| VDAC1 | NM_003374/NM_003150 | 4 |
| WSB2 | NM_018639 | 1 |
| ZDHHC9 | NM_001008222 | 1 |
| ZNF878 | NM_001080404 | 4 |

### Impact of Intermediate Metabolism on CDDP Responses.

Inventors observed that pyridoxal kinase (PDXK) and aldehyde dehydrogenase family 7, member A1 (ALDH7A1, an evolutionarily conserved protein also known as antiquitin), two enzymes involved in vitamin B6 metabolism (FIG. 5A), were both found in cluster 4 (FIG. 4A), suggesting that they act on similar pathway to modulate CDDP responses. Therefore, they decided to investigate the impact of the enzymatically active form of vitamin B6 (pyridoxal-5-phosphate, PLP) and its closest precursor (the B6 vitamer pyridoxine, PN) on CDDP responses.

PDXK, converts vitamin B6 precursors into their enzymatically active derivative, PLP {Lee, 2000}. ALDH7A1 metabolizes L-2-aminoadipate 6-semialdehyde (AAS, also known as piperideine-6-carboxylate) which reacts with PLP by Knoevenagel condensation {Mills, 2006}. The depletion of ALDH7A1 hence indirectly causes a reduction in PLP levels. Acute addition of PN (but not that of PLP, which cannot cross the plasma membrane) increased the intracellular PN concentration (FIG. 5B) and exacerbated CDDP-induced cell death, as quantified by DiOC₆(3)/PI co-staining (FIG. 5C) or by measuring impedance caused by attached, viable cells (FIG. 8F). Chronic treatment with increasing amounts of B6 vitamer sensitized both NSCLC (FIG. 5D) and yeast cells (FIG. 5E) to CDDP, but the same effect could not be observed when yeast cells were killed by cadmium (FIG. 5E). Inventors then transfected A549 cells with a series of siRNA targeting B6-relevant enzymes (FIG. 5F) including PDXK, ALDH7A1, as well as PDX phosphatase (PDXP, which functionally antagonizes PDXK, FIG. 5A) and tested their response to CDDP. As expected, PDXK or ALDH7A1 depletion reduced the cytotoxic effect of CDDP, while the knockdown of PDXP provided chemosensitization (FIG. 5G). The effects of PN were lost upon PDXK depletion (FIG. 5H), in accord with the notion that PN must be converted into PLP to become active (FIG. 5I). PLP is the enzymatic co-factor of cysteine conjugate-β lyase (CCBL1), an enzyme that has previously been implicated in the renal toxicity of CDDP {Zhang, 2003}. siRNA-mediated depletion of CCBL1 (FIG. 5J) or addition of the CCBL1 inhibitor aminooxyacetic acid (AOAA) reduced the toxicity of CDDP in both A549 (FIG. 5K) and yeast cells (FIG. 5L). In yeast, the knockout of vitamin B6-related genes (sno1, snz1) as well as that of the bona fide PDXK ortholog bud17, rescued from CDDP (but not cadmium)-induced cell death (FIG. 8G). PN exerted chemosensitizing effects on three distinct CDDP-resistant A549 clones, on four additional NSCLC cell lines, as well as on cervical carcinoma, osteosarcoma and wild type (but not TP53^{-/-}) colorectal cancer cells (FIG. 10A-C).

Non-oxidized glutathione (GSH) or N-acetylcysteine (NAC) blocked the apoptosis-inducing effect of CDDP (irrespective of the presence of PN), be it quantified by DiOC₆(3)/PI co-staining (FIG. 8G) or as DNA loss (sub-G₁ peak in FIG. 6A). However, NAC failed to reverse the cell cycle arrest induced by CDDP, an effect that was not affected by the presence of PN (FIG. 6A, FIG. 8H). Therefore, they further focused on the cell death-sensitizing effects of PN. In the presence of PN, cells treated by a sub-apoptotic concentration of CDDP exhibited an increased activating phosphorylation of TP53 on serines 15 and 46 (note that TP53 is in the same epistatic cluster as PDXK, FIG. 4, and that TP53 is required for PN-mediated sensitization to CDDP, FIG. 10C), and an increased activation of caspases-3 and -9 (FIG. 6B). Inhibition of caspases, indeed, reduced killing by CDDP alone or in combination with PN, although it did not completely abrogate PN-mediated chemosensitization (FIG. 8G). PN significantly increased CDDP-DNA adducts, as quantified by immunofluorescence staining of cells cultured in the presence of CDDP (FIG. 6C) followed by automated image analysis (FIG. 6D). Accordingly, two signs of an ongoing DNA damage response to CDDP, namely the activating phosphorylation (on Ser1981) of the ataxia telangiectasia mutated (ATM) kinase and the phosphorylation on Ser140 of the ATM substrate histone 2AX (H2AX) of SEQ ID NO: 188, were increased in the presence of PN (FIG. 14 A-D).

Inventors also investigated another druggable metabolic pathway and surprisingly discovered that both the knockdown and pharmacological inhibition (with Orlistat®) of LIPC enhanced the cytotoxiticy of CDDP against A549 cells in vitro (FIG. 1). In line with these observations, systemic injections of Orlistat® also advantageously augmented the antitumor effects of CDDP-based chemotherapy in vivo, in A549 cells growing on immunodeficient Swiss nude mice, as well as in murine Lewis lung carcinoma cells transplanted into immunocompetent C57BL/6 mice (FIG. 6E).

### Prognostic and Predictive Impact of PDXK and LIPC on Non-Small Cell Lung Cancer.

To finally validate the results of the siRNA screen on human NSCLC tumor samples, inventors took advantage of 122 operable NSCLC specimens that were or were not treated with adjuvant CDDP-based chemotherapy (Table 6). Sections of each tumor were stained with antibodies specific for ALDH7A1, ALK, BCL2L1, LIPC, PDXK, PDXP and WSB2. As quantified by immunohistochemistry (FIG. 7A, FIG. 11), the expression levels of each of these proteins failed to exhibit a major positive or negative correlation among each other (FIG. 12). Tumor samples were then arbitrarily divided into two groups with high and low expression of each of the proteins of interest, and their impact on overall survival (OS) and disease-free survival (DFS) was determined, either for all patients or for subgroups that received chemotherapy or were left untreated. The protein expression levels of ALDH7A1, ALK, BCL2L1, PDXP and WSB2 had no impact on OS, DFS or therapeutic responses (FIG. 13).

In contrast, low expression of PDXK had a significant negative impact on DFS (FIG. 7B), irrespective of whether the patients were treated with CDDP or not. Indeed the expression of PDXK did not affect the therapeutic response to CDDP as estimated with survival curves based on a Cox proportional hazard model (FIG. 7C). Thus PDXK constitutes a prognostic biomarker. The absence of LIPC had a significant negative effect on OS when all patients were analyzed together (FIG. 7D). However, LIPC⁻ tumors tended to respond to chemotherapy, while LIPC⁺ ones exhibited a paradoxical behavior in thus far that CDDP-treated patients tended to show a higher frequency of events than untreated patients (FIG. 7E). The interaction between LIPC expression levels and treatment outcome were significant, indicating that LIPC constitutes a predictive biomarker.

TABLE 6
Patient characteristics.
The cohort of patients included in this study is classified
with respect to age, gender, smoking status, histological
parameters and treatment with adjuvant chemotherapy.
N (%)*

| | | |
|---|---|---|
| Adjuvant chemotherapy | No | 64 (52.45) |
| | Yes | 58 (47.55) |
| Histology | ADC | 56 (45.90) |
| | LCC | 13 (10.66) |
| | SCC | 50 (40.98) |
| | Others | 3 (2.46) |
| Gender | Men | 33 (27.05) |
| | Women | 89 (72.95) |
| Smoking status | Current | 47 (38.52) |
| | Former | 66 (54.10) |
| | Never | 7 (5.74) |
| | Unknown | 2 (1.64) |

Median (range) years**

| | |
|---|---|
| Age | 63 (41-85) |
| Follow-up | 2.7 (0.5-8.3) |

*= absolute number (percentage);
**= median (range);
ADC, adenocarcinoma;
LCC, large cell carcinoma;
SCC, squamous cell carcinoma.

### Concluding Remarks

In this work, inventors identified in particular 85 CRMs by a non-saturating genome-wide siRNA screen and several subsequent and variant rounds of validation. These include known apoptosis regulators (negative: MCL1, BCL2L1, TFF1; positive: APAF1) {Bossenmeyer-Pourie, 2002; Kroemer, 2007}, pro-survival proteins (IL6R), as well as multiple proteins involved in intermediate metabolism (LIPC; homogentisate 1,2-dioxygenase, HGD; high density lipoprotein binding protein, HDLBP; fructosamine-3-kinase-related protein, FN3KRP; COX subunits). A majority of the 85 transcripts/proteins had cytoprotective properties, meaning that their knockdown or their pharmacological inhibition enhances cisplatin-induced killing. Inventors herein demonstrate that these cytoprotective proteins constitute novel targets for chemosensitization. Epistatic analyses indicate that dual interventions on at least two among these transcripts/proteins induce synergistic chemosensitizing effects. In this example, inventors in particular showed that Orlistat®, an inhibitor of LIPC, can improve the efficacy of cisplatin in a preclinical setting, in mice bearing murine or human NSCLC.

The siRNA screening also led to the identification of multiple transcripts/proteins whose depletion reduces cisplatin responses, implying that they directly or indirectly contribute to CDDP toxicity. Based on the obtained results, inventors identified ALK inhibitors as detrimental, when used in combination with CDDP, for the therapy of NSCLC. Moreover, the knockdown of two subunits of the respiratory chain complex COX or chemical COX inhibition reduces the toxicity of cisplatin. Thus, a progressive reduction of oxidative phosphorylation, as it occurs during oncogenesis and tumor progression {Kroemer, 2008}, might reduce CDDP responses as a ‘natural’ resistance mechanism. The knockdown of PDXK (and that of another vitamin B6-related enzyme, ALDH7A1) reduced the cytotoxic activity of CDDP, leading to the discovery that PDXP depletion (the phosphatase that functionally antagonizes PDXK) can stimulate cisplatin-induced tumor cell killing. In line with these observations, inventors found that exogenous administration of a cell-permeable vitamin B6 precursor, PN, sensitized cultured NSCLC cells to CDDP, provided that PDXK was present.

The transcription of most of the 85 CRMs reported here was not affected by CDDP (FIG. 2 and Table 3). Indeed, the percentage of CRMs whose mRNA levels were significantly modulated by CDDP (<10%) was not higher than that affected by the general stress mediator ceramide. Moreover, most CRMs affected the CDDP response (and that of carboplatin as well as camptothecin) in a specific fashion, yet had discordant effects on ceramide, cadmium, pro-apoptotic triggers (such as staurosporine or thapsigargin) or unrelated chemotherapeutics (such as betulinic acid) (FIG. 3). In contrast, the 85 CRMs had similar effects on a range of NSCLC (but not on colorectal cancer) cells.

Driven by the availability of monoclonal antibodies suitable for immunohistochemistry, inventors determined the expression level of 7 CRMs on a panel of 120 NSCLC biopsies. Although there was some degree of heterogeneity in their expression levels, 5 out of these 7 markers had no predictive or prognostic impact, implying that the abundance of these 5 proteins does not have sufficient biological impact to affect the fate of a heterogeneous collection of NSCLC patients. However, 2 proteins, in particular, that they characterized did have a prognostic impact.

Reduced PDXK expression negatively affected DFS. In this context, inventors highlight the paradox of the results provided by Johansson {Johansson, 2010} showing that circulating vitamin B6 levels negatively correlate with the risk of developing clinically manifest NSCLC, and herein demonstrate that vitamin B6 itself—and the enzyme that renders it bioactive—have a profound impact on the biology of NSCLC. Although PDXK affected prognosis, it had no predictive value, meaning that patients expressing high or low levels of PDXK responded similarly to adjuvant chemotherapy. As a possibility, the levels of PDXK may reflect profound differences in the natural history of NSCLC that largely supersede its hypothetical CRM effect in importance.

No expression of LIPC had a negative impact on OS. At first glance, this result appears paradoxical because the depletion of LIPC had chemosensitizing properties in vitro and its pharmacological inhibition improved the CDDP response in vitro and in vivo. Only upon further analyses, when patient groups were divided into those who received chemotherapy and those who did not, it turned out that the clinical impact of LIPC expression concords with its biological properties. The absence of LIPC, which among untreated patients have a negative prognostic impact, predict that adjuvant chemotherapy improves patient survival. In contrast, LIPC expression predicts that adjuvant chemotherapy has no beneficial effect and actually adversely affects patient survival.

### Example 2

### Prognostic Impact of PDXK on Non-Small Cell Lung Cancer

To investigate the impact of PDXK expression on NSCLC, inventors developed an immunohistochemical staining method that specifically detects PDXK (as demonstrated by the fact that siRNA-mediated depletion of PDXK results in the complete loss of the signal) (FIG. 18 D, E) and applied it to 120 operable NSCLC specimens (Table 7). Sections of each tumor were stained with antibodies specific for PDXK (FIG. 18F) or PDXP (FIG. 11A). Upon immunohistochemical analysis (FIG. 18 F, G), tumor samples were arbitrarily divided into two groups with high and low PDXK and PDXP expression, and the impact on overall survival (OS) and disease-free survival (DFS) was determined. Whereas no correlation was find between PDXP abundance and either OS or DFS (FIGS. 11A and 13G), low expression levels of PDXK had a significant negative impact on DFS (FIG. 18G). Thus, PDXK protein expression constitutes a prognostic biomarker.

TABLE 7
Patient characteristics.
N (%)*

| | | |
|---|---|---|
| Adjuvant chemotherapy | No | 63 (52.50) |
| | Yes | 57 (47.50) |
| Histology | ADC | 56 (46.70) |
| | LCC | 49 (40.80) |
| | Others | 15 (12.50) |
| Gender | Men | 88 (73.30) |
| | Women | 32 (26.70) |
| Smoking status | Current | 47 (39.20) |
| | Former | 50 (41.70) |
| | Never | 7 (5.80) |
| | Unknown | 16 (13.30) |

Median (range) years**

| | |
|---|---|
| Age | 62.9 (40.9-84.7) |
| Follow-up | 4.72 |

*= absolute number (percentage);
**= median (range);
ADC, adenocarcinoma;
LCC, large cell carcinoma.

### Example 3

### Mechanism of Vitamin B6-Mediated Chemosensitization

To confirm the molecular mechanisms underlying vitamin B6-mediated chemosensitization, inventors performed targeted metabolic determinations and metabolic profiling. They found that, in pre-apoptotic conditions, NSCLC cells treated with CDDP and PN exhibited a bioenergetic defect (measured in terms of Atkinson's energy charge=[ATP]+0.5 [ADP]/[ATP]+[ADP]+[AMP]) (Atkinson, 1968) that was not present in cells incubated with either of these compounds alone (FIG. 15A and FIG. 16). Moreover, A549 cells succumbed to CDDP while manifesting a decrease in the intracellular levels of GSH (FIG. 15B, 15C) as well as of three metabolites with a MW of approximately 161, 203 and 217 Da, which were identified as carnitine, acetylcarnitine and propionylcarnitine, respectively (FIG. 17). This decrease in GSH and redox-relevant metabolites was largely exacerbated by PN (FIG. 15B, 15C and FIG. 17A-C) and only GSH (FIG. 8G) (but not carnitine metabolites, FIG. 17D, 17E) protected cells against the cytotoxic effect of CDDP plus PN. While extracellular GSH had no significant impact on intracellular GSH levels (FIG. 15D), GSH strongly inhibited the accumulation of CDDP within the cells, consistent with its marked cytoprotective effects (FIG. 15E). Moreover, intracellular CDDP levels were significantly augmented by PN in a PDXK-dependent fashion (FIG. 15E, 15F). These results indicate that vitamin B6 exerts chemosensitizing effects by accruing the intracellular accumulation of CDDP.

Systemic injections of PN augmented the antitumor effects of CDDP-based chemotherapy in vivo, in murine Lewis lung carcinoma cells transplanted into immunocompetent C57BL/6 mice (FIG. 18A) confirming that the chemosensitizing potential of PN is preserved in vivo.

To validate this observation in another in vivo experimental setting, inventors generated A549 cell clones stably expressing a short hairpin RNA (shRNA) for the downregulation of PDXK. These clones responded to CDDP as expected in vitro (FIG. 18C), yet failed to generate sizeable tumors in immunodeficient Swiss nude mice with the same efficiency as their PDXK-proficient counterparts (FIG. 18B). Moreover, established PDXK-deficient tumors exhibited strikingly reduced growth rates as compared to tumors originating from control cells (FIG. 18B). These results demonstrate that vitamin B6 metabolism not only affect the chemotherapeutic response of NSCLC cells but also affects NSCLC oncogenesis.

### REFERENCES

Atkinson D E. The energy charge of the adenylate pool as a regulatory parameter. Interaction with feedback modifiers. Biochemistry. 1968; 7(11):4030-4.
Beers, M. H. (2008). Lung Carcinoma. In The Merck manual of diagnosis and therapy, R. S. Porter, and T. V. Jones, eds. (Rahway, Merck & Co., Inc.), p. 2992.
Bertolini, G., Roz, L., Perego, P., Tortoreto, M., Fontanella, E., Gatti, L., Pratesi, G., Fabbri, A., Andriani, F., Tinelli, S., et al. (2009). Highly tumorigenic lung cancer CD 133+ cells display stem-like features and are spared by cisplatin treatment. Proc Natl Acad Sci USA 106, 16281-16286.
Bossenmeyer-Pourie, C., Kannan, R., Ribieras, S., Wendling, C., Stoll, I., Thim, L., Tomasetto, C., and Rio, M. C. (2002). The trefoil factor 1 participates in gastrointestinal cell differentiation by delaying G1-S phase transition and reducing apoptosis. J Cell Biol 157, 761-770.
Boyd, V. L. and Zon, G. (2004). Bisulfite conversion of genomic DNA for methylation analysis: protocol simplification with higher recovery applicable to limited samples and increased throughput. Anal Biochem. 326, 278-280.
Breen, D., and Barlesi, F. (2008). The place of excision repair cross complementation 1 (ERCC1) in surgically treated non-small cell lung cancer. Eur J Cardiothorac Surg 33, 805-811.
Castedo, M., Coquelle, A., Vivet, S., Vitale, I., Kauffmann, A., Dessen, P., Pequignot, M. O., Casares, N., Valent, A., Mouhamad, S., et al. (2006). Apoptosis regulation in tetraploid cancer cells. EMBO J. 25, 2584-2595.
Cosaert, J., and Quoix, E. (2002). Platinum drugs in the treatment of non-small-cell lung cancer. Br J Cancer 87, 825-833.
Dai, C. H., Li, J., Shi, S. B., Yu, L. C., Ge, L. P., and Chen, P. (2010). Survivin and Smac gene expressions but not livin are predictors of prognosis in non-small cell lung cancer patients treated with adjuvant chemotherapy following surgery. Jpn J Clin Oncol 40, 327-335.
de La Motte Rouge, T., Galluzzi, L., Olaussen, K. A., Zermati, Y., Tasdemir, E., Robert, T., Ripoche, H., Lazar, V., Dessen, P., Harper, F., et al. (2007). A novel epidermal growth factor receptor inhibitor promotes apoptosis in non-small cell lung cancer cells resistant to erlotinib. Cancer Res 67, 6253-6262.
Dzagnidze, A., Katsarava, Z., Makhalova, J., Liedert, B., Yoon, M. S., Kaube, H., Limmroth, V. and Thomale J. (2007) Repair capacity for platinum-DNA adducts determines the severity of cisplatin-induced peripheral neuropathy. J. Neurosci. 27, 9451-9457.
Frommer, M., McDonald, L. E., Millar, D. S., Collis, C. M., Watt, F., Grigg, G. W., Molloy, P. L. and Paul, C L. (1992). A genomic sequencing protocol that yields a positive display of 5-methylcytosine residues in individual DNA strands. Proc Natl Acad Sci USA. 89, 1827-1831.
Galluzzi, L., Zamzami, N., de La Motte Rouge, T., Lemaire, C., Brenner, C. and Kroemer, G. (2007). Methods for the assessment of mitochondrial membrane permeabilization in apoptosis. Apoptosis. 12, 803-813.
Galluzzi, L., Vitale, I., Kepp, O., Séror, C., Hangen, E., Perfettini, J. L., Modjtahedi, N. and Kroemer, G. (2008). Methods to dissect mitochondrial membrane permeabilization in the course of apoptosis. Methods Enzymol. 442, 355-374.
Galluzzi, L., Aaronson, S. A., Abrams, J., Alnemri, E. S., Andrews, D. W., Baehrecke, E. H., Bazan, N. G., Blagosklonny, M. V., Blomgren, K., Borner, C. et al (2009). Guidelines for the use and interpretation of assays for monitoring cell death in higher eukaryotes. Cell Death Differ. 16, 1093-1107.
Hoffmann, J., Vitale, I., Buchmann, B., Galluzzi, L., Schwede, W., Senovilla, L., Skuballa, W., Vivet, S., Lichtner, R. B., Vicencio, J. M., et al. (2008). Improved cellular pharmacokinetics and pharmacodynamics underlie the wide anticancer activity of sagopilone. Cancer Res 68, 5301-5308.
Jemal, A., Thun, M. J., Ries, L. A., Howe, H. L., Weir, H. K., Center, M. M., Ward, E., Wu, X. C., Eheman, C., Anderson, R., et al. (2008). Annual report to the nation on the status of cancer, 1975-2005, featuring trends in lung cancer, tobacco use, and tobacco control. J Natl Cancer Inst 100, 1672-1694.
Johansson, M., Relton, C., Ueland, P. M., Vollset, S. E., Midttun, O., Nygard, O., Slimani, N., Boffetta, P., Jenab, M., Clavel-Chapelon, F., et al. (2010). Serum B vitamin levels and risk of lung cancer. JAMA 303, 2377-2385.
Kancha, R. K., von Bubnoff, N., Peschel, C., and Duyster, J. (2009). Functional analysis of epidermal growth factor receptor (EGFR) mutations and potential implications for EGFR targeted therapy. Clin Cancer Res 15, 460-467.
Kroemer, G., Galluzzi, L., and Brenner, C. (2007). Mitochondrial membrane permeabilization in cell death. Physiol Rev 87, 99-163.
Kroemer, G., and Pouyssegur, J. (2008). Tumor cell metabolism: cancer's Achilles' heel. Cancer Cell 13, 472-482.
Lee, H. S., Moon, B. J., Choi, S. Y., and Kwon, O. S. (2000). Human pyridoxal kinase: overexpression and properties of the recombinant enzyme. Mol Cells 10, 452-459.
Liedert, B., Pluim, D., Schellens, J. and Thomale, J. (2006) Adduct-specific monoclonal antibodies for the measurement of cisplatin-induced DNA lesions in individual cell nuclei. Nucleic Acids Res. 34, e47.
Loriot, Y., Mordant, P., Dorvault, N., De la motte Rouge, T., Bourhis, J., Soria, J. C. and Deutsch, E. (2010). BMS-690514, a VEGFR and EGFR tyrosine kinase inhibitor, shows anti-tumoural activity on non-small-cell lung cancer xenografts and induces sequence-dependent synergistic effect with radiation. Br J Cancer. 103, 347-53.
Madeo, F., Herker, E., Maldener, C., Wissing, S., Lächelt, S., Herlan, M., Fehr, M., Lauber, K., Sigrist, S. J., Wesselborg, S. et al. (2002). A caspase-related protease regulates apoptosis in yeast. Mol. Cell. 9, 911-917.
Mandic, A., Hansson, J., Linder, S., and Shoshan, M. C. (2003). Cisplatin induces endoplasmic reticulum stress and nucleus-independent apoptotic signaling. J Biol Chem 278, 9100-9106.
Martin, L. P., Hamilton, T. C., and Schilder, R. J. (2008). Platinum resistance: the role of DNA repair pathways. Clin Cancer Res 14, 1291-1295.
Midttun, Ø., Hustad, S, and Ueland, P. M. (2009) Quantitative profiling of biomarkers related to B-vitamin status, tryptophan metabolism and inflammation in human plasma by liquid chromatography/tandem mass spectrometry. Rapid Commun Mass Spectrom. 23, 1371-1379.
Mills, P. B., Struys, E., Jakobs, C., Plecko, B., Baxter, P., Baumgartner, M., Willemsen, M. A., Omran, H., Tacke, U., Uhlenberg, B., et al. (2006). Mutations in antiquitin in individuals with pyridoxine-dependent seizures. Nat Med 12, 307-309.
Mondragon, L., Galluzzi, L., Mouhamad, S., Orzaez, M., Vicencio, J. M., Vitale, I., Moure, A., Messeguer, A., Perez-Paya, E., and Kroemer, G. (2009). A chemical inhibitor of Apaf-1 exerts mitochondrioprotective functions and interferes with the intra-5-phase DNA damage checkpoint. Apoptosis 14, 182-190.
Mouhamad, S., Galluzzi, L., Zermati, Y., Castedo, M. and Kroemer, G. (2007). Apaf-1 Deficiency Causes Chromosomal Instability. Cell Cycle. 6, 3103-3107.
Olaussen, K. A., Dunant, A., Fouret, P., Brambilla, E., Andre, F., Haddad, V., Taranchon, E., Filipits, M., Pirker, R., Popper, H. H., et al. (2006). DNA repair by ERCC1 in non-small-cell lung cancer and cisplatin-based adjuvant chemotherapy. N Engl J Med 355, 983-991.
Osoegawa, A., Yoshino, I., Tanaka, S., Sugio, K., Kameyama, T., Yamaguchi, M., and Maehara, Y. (2004). Regulation of p27 by S-phase kinase-associated protein 2 is associated with aggressiveness in non-small-cell lung cancer. J Clin Oncol 22, 4165-4173.
Ota, S., Ishii, G., Goto, K., Kubota, K., Kim, Y. H., Kojika, M., Murata, Y., Yamazaki, M., Nishiwaki, Y., Eguchi, K., et al. (2009). Immunohistochemical expression of BCRP and ERCC1 in biopsy specimen predicts survival in advanced non-small-cell lung cancer treated with cisplatin-based chemotherapy. Lung Cancer 64, 98-104.
Rodríguez-Navarro, S., Llorente, B., Rodríguez-Manzaneque, M. T., Ramne, A., Uber, G., Marchesan, D., Dujon, B., Herrero, E., Sunnerhagen, P. and Pérez-Ortín, J. E. (2002). Functional analysis of yeast gene families involved in metabolism of vitamins B1 and B6. Yeast. 19, 1261-1276.
Saeed, A I, Sharov, V, White, J, L1, J, Liang, W, Bhagabati, N, Braisted, J, Klapa, M, Currier, T, Thiagarajan, M, et al. (2003). TM4: a free, open-source system for microarray data management and analysis. Biotechniques 34 (2):374-8.
Schwarzer, G. (2007) Meta: An R Package for Meta-Analysis. R News, 7/3, 40-45.
Seve, P., and Dumontet, C. (2005). Chemoresistance in non-small cell lung cancer. Curr Med Chem Anticancer Agents 5, 73-88.
Soda, M., Choi, Y. L., Enomoto, M., Takada, S., Yamashita, Y., Ishikawa, S., Fujiwara, S., Watanabe, H., Kurashina, K., Hatanaka, H., et al. (2007). Identification of the transforming EML4-ALK fusion gene in non-small-cell lung cancer. Nature 448, 561-566.
Streit, M., Riccardi, L., Velasco, P., Brown, L. F., Hawighorst, T., Bornstein, P. and Detmar, M. (1999) Thrombospondin-2: a potent endogenous inhibitor of tumor growth and angiogenesis. Proc Natl Acad Sci USA. 96, 14888-14893.
Tajeddine, N., Galluzzi, L., Kepp, O., Hangen, E., Morselli, E., Senovilla, L., Araujo, N., Pinna, G., Larochette, N., Zamzami, N., et al. (2008). Hierarchical involvement of Bak, VDAC1 and Bax in cisplatin-induced cell death. Oncogene 27, 4221-4232.
Vicencio, J. M., Ortiz, C., Criollo, A., Jones, A. W., Kepp, O., Galluzzi, L., Joza, N., Vitale, I., Morselli, E., Tailler, M., et al. (2009). The inositol 1,4,5-trisphosphate receptor regulates autophagy through its interaction with Beclin 1. Cell Death Differ 16, 1006-1017.
Vitale, I., Galluzzi, L., Vivet, S., Nanty, L., Dessen, P., Senovilla, L., Olaussen, K. A., Lazar, V., Prudhomme, M., Golsteyn, R. M. et al. (2007). Inhibition of Chk1 kills tetraploid tumor cells through a p53-dependent pathway. PLoS One 2, e1337.
Vitale, I., Senovilla, L., Galluzzi, L., Criollo, A., Vivet, S., Castedo, M., Koemer, G., et al. (2008). Chk1 inhibition activates p53 through p38 MAPK in tetraploid cancer cells. Cell Cycle 7, 1956-61.
Zermati Y., Mouhamad S., Stergiou L., Besse B., Galluzzi L., Boehrer S., Pauleau A. L., Rosselli F., D'Amelio M., Amendola R. et al. (2007). Nonapoptotic role for Apaf-1 in the DNA damage checkpoint. Mol. Cell. 28, 624-37.
Zhang, L., and Hanigan, M. H. (2003). Role of cysteine S-conjugate beta-lyase in the metabolism of cisplatin. J Pharmacol Exp Ther 306, 988-994.
Zischka, H., Larochette, N., Hoffmann, F., Hamöller, D., Jägemann, N., Lichtmannegger, J., Jennen, L., Müller-Höcker, J., Roggel, F., Göttlicher, M. et al. (2008). Electrophoretic analysis of the mitochondrial outer membrane rupture induced by permeability transition. Anal Chem. 80, 5051-5058.

1-19. (canceled)

20. An in vitro method of assessing or monitoring the sensitivity of a subject having a tumor to a chemotherapy, which method comprises a step of determining, in a biological sample of said subject, the absence, presence or expression level of the expression product of at least one gene selected from the genes identified in Table I, in at least one gene selected from the genes identified in Table 1, thereby assessing or monitoring whether the subject having a tumor is responsive or resistant to the chemotherapy.

21. The method according to claim 20, wherein the method further comprises a step of comparing the expression level of the at least one gene in the biological sample of the subject to a reference expression level.

22. The method according to claim 20, wherein the at least one gene is the hepatic lipase (LIPC) gene.

23. The method according to claim 22, wherein the presence of LIPC in the biological sample, is indicative of a resistance of the subject to the chemotherapy.

24. A method of selecting an appropriate treatment of a cancer for a subject having a tumor, which method comprises a step of determining the presence or absence of LIPC in a biological sample of said subject, the absence of LIPC in the biological sample being the indication that a chemotherapy will be efficient in the subject, the presence of LIPC in the biological sample being the indication that a chemotherapy will be detrimental to the subject.

25. A method of assessing the prognosis of a cancer in a subject, the method comprising a step of determining, in a biological sample of said subject, the absence, presence or expression level of a compound selected from vitamin B6, pyridoxal (PL), pyridoxal-5-phosphate (PLP), pyridoxamine (PM), pyridoxamine-5′-phosphate, pyridoxine (PN), pyridoxine-5′-phosphate, L-2-aminoadipate and L-2-aminoadipate 6-semialdehyde, and/or of the expression product of at least one gene selected from the genes identified in Table I, or a derivative product thereof, thereby assessing the prognosis of the cancer in the subject.

26. The method according to claim 25, wherein the method further comprises a step of comparing the expression level of the at least one gene in the biological sample of the subject to a reference expression level.

27. The method according to claim 25, wherein the at least one gene is the hepatic lipase (LIPC) gene and the presence of LIPC is indicative of a favourable prognosis of the cancer in the subject.

28. The method according to claim 25, wherein the at least one gene is the hepatic lipase (LIPC) gene and the absence of LIPC is indicative of an unfavourable prognosis of the cancer in the subject.

29. The method according to claim 26, wherein the at least one gene is the pyridoxal kinase (PDXK) gene and an overexpression thereof when compared to a reference expression level is indicative of a favourable prognosis of the cancer in the subject.

30. The method according to claim 26, wherein the at least one gene is the pyridoxal kinase (PDXK) gene and the non-expression thereof or a reduced expression thereof when compared to a reference expression level is indicative of an unfavourable prognosis of the cancer in the subject.

31. The method according to claim 20, wherein the chemotherapy comprises administering an alkylating agent or an alkylating-like agent.

32. The method according to claim 20, wherein the cancer is selected from non-small cell lung cancer (NSCLC), small cell lung cancer (SCLC), head and neck cancer, cervical carcinoma, ovarian cancer, osteosarcoma, melanoma or colorectal cancer.

33. The method according to claim 20, wherein the biological sample of the subject is selected from a tumor sample, a blood sample, a serum sample and a bronchoalveolar lavage (BAL).

34. A method of selecting an appropriate treatment of a cancer for a subject having a tumor, which method comprises a step of determining the expression level of a compound selected from PDXK, PDXP or aldehyde dehydrogenase 7 family, member A1 (ALDH7A1) in a biological sample of said subject, the non-expression thereof or a reduced expression thereof, when compared to a reference expression level, being the indication that a compound selected from vitamin B6, pyridoxal (PL), pyridoxal-5-phosphate (PLP), pyridoxamine (PM), pyridoxamine-5′-phosphate, pyridoxine (PN), or pyridoxine-5′-phosphate (PNP), should be administered to the patient alone or together with a conventional treatment of cancer.

35. A method of selecting an appropriate treatment of a cancer for a subject having a tumor, which method comprises a step of determining the presence, absence or expression level of LIPC in a biological sample of said subject, the presence of LIPC in the biological sample, or an overexpression thereof when compared to a reference expression level, being the indication that Orlistat should be administered to the patient together with a conventional treatment of cancer.

36. A kit for assessing or monitoring the sensitivity of a subject having a tumor to a chemotherapy, or the prognosis of a cancer in a subject, wherein the kit comprises detection means selected from the group consisting of a pair of primers, a probe, at least one antibody specific to the expression product of a gene selected from the genes identified in Table I or to a derivative product thereof, and a DNA chip, and, optionally, a leaflet providing the wild-type sequence of the gene and/or the control quantitative expression value corresponding to the expression product of the gene in a control population and/or corresponding to a derivative product thereof.

37. A DNA chip comprising a solid support which carries nucleic acids that are specific to at least two genes selected from the genes identified in Table I.

38. A method for screening or identifying a compound suitable for improving the treatment of a cancer in a subject having a tumor, said method comprising determining the ability of a test compound to modify the expression of at least one gene selected from the genes identified in Table I, or compensate an abnormal expression thereof.
